(19) United States
(12) Patent Application Publication
(10) Pub. No.: US 2024/0228668 A1
(43) Pub. Date: Jul. 11, 2024

(54) COMPOSITIONS AND METHODS COMPRISING ANTIBODIES THAT BIND TO COVALENT PEPTIDE CONJUGATES

(71) Applicant: New York University, New York, NY (US)

(72) Inventors: Shohei KOIDE, New York, NY (US); Benjamin NEEL, New York, NY (US); Carmine FEDELE, New York, NY (US); Kai Wen TENG, Flushing, NY (US); Akiko KOIDE, New York, NY (US); Takamitsu HATTORI, West New York, NJ (US); Lorenzo MASO, New York, NY (US)

(21) Appl. No.: 18/547,623

(22) PCT Filed: Feb. 28, 2022
(86) PCT No.: PCT/US2022/018171

§ 371 Date: Aug. 23, 2023

Related U.S. Application Data
(60) Provisional application No. 63/253,499, filed on Oct. 7, 2021.
(60) Provisional application No. 63/154,627, filed on Feb. 26, 2021.

(51) Int. Cl. C07K 16/44; A61K 47/68; C07K 16/28

(52) U.S. Cl. CPC C07K 16/44; A61K 47/6849; C07K 16/2833; C07K 2317/56; C07K 2317/626

(57) ABSTRACT

Provided are compositions and methods that include binding partners that bind with specificity to target sites on proteins or peptides that comprise a covalently attached molecule. The binding partners are provided as antibodies and antibody derivatives that specifically bind to proteins and peptides that have been covalently modified by attachment of a molecule, such as a drug. The binding partners can bind with specificity to covalently modified peptides when presented in the context of a major histocompatibility complex (MHC). Uses of the compositions and methods for prophylaxis or therapy of disorders are also provided.

### CROSS REFERENCE TO RELATED APPLICATIONS

This application claims priority to U.S. provisional patent application No. 63/154,627, filed Feb. 26, 2021, and U.S. provisional patent application No. 63/253,499, filed Oct. 7, 2021, the entire disclosures of each of which are incorporated herein by reference.

### STATEMENT REGARDING FEDERALLY SPONSORED RESEARCH

This invention was made with government support under grant nos. CA194864, CA267362 and CA049152 awarded by the National Institutes of Health. The government has certain rights in the invention.

### SEQUENCE LISTING

The instant application contains a Sequence Listing, which has been submitted electronically in ASCII format and is hereby incorporated by reference in its entirety. Said ASCII copy, created on Feb. 24, 2022, is named 58636_00482_SL.txt, and is 212,998 bytes in size.

### BACKGROUND

There is an ongoing and unmet need for agents that can bind to targets that include drugs that are covalently bound to proteins or peptides. In particular, there is a need to improve the efficacy of targeted therapy and also to increase tumor immunogenicity and the efficacy of immune therapy against cancer driven by intracellular oncogenes. The disclosure is pertinent to these needs.

### BRIEF SUMMARY

The present disclosure provides compositions and methods that include binding partners that bind with specificity to target sites on proteins or peptides that comprise a covalently attached molecule. It is believed that this is the first disclosure of binding partners with this binding function. The disclosure illustrates this approach using binding partners in the form of numerous antibodies and antibody derivatives that specifically bind to proteins and peptides that have been covalently modified by attachment of a molecule, wherein the molecules are illustrated by a variety of drugs. Further, the disclosure demonstrates binding partners that bind with specificity to peptides that have been covalently modified by attachment of a small molecule drug are specific for the described covalently modified peptides when presented in the context of a human leukocyte antigen (HLA), wherein HLA is a representative example of a major histocompatibility complex (MHC). Thus, binding partners that are specific for peptide-drug conjugates in an HLA complex are demonstrated. The disclosure includes polynucleotides encoding the described binding partners and cells that are modified to express the binding partners. The disclosure includes diagnostic, prophylactic and therapeutic approaches using the binding partners.

### BRIEF DESCRIPTION OF THE FIGURES

FIG. 1. Graphs depicting results of phage enzyme-linked immunosorbent assay (ELISA) of phage-displayed antibody clones. Binding to KRAS^{G12C}-GDP and KRAS^{G12C}-GDP-ARS-1620 conjugate was determined. For each clone, the bars in the graph are, from left to right, Buffer, G12C-GDP-ARS, and G12C-GDP.

FIG. 2. Graph showing 12C-ARS Fab59 binding to KRAS^{G12C} in GTPγS- or GDP-bound nucleotide state with or without ARS-1620 was characterized using the bead binding assay.

FIG. 3. Graphs showing that 12C-ARS-Fab59 specifically binds KRAS^{G12C}-GDP conjugated to ARS-1620.

FIG. 4. Shown in (A) is data obtained using 12C-ARS-Fab59 to measure ARS-1620/KRAS^{G12C} adducts by pull-down assays from lysates prepared from cell lines. Shown in (B) are immunoblots of whole cell lysates and 12C-ARS Fab-pull-downs (PD) from RAS-less MEFs reconstituted with the indicated KRAS mutants (4A) and KCP (Kras^{G12C}; Tp53^{R172H}; Pdx-Cre) mouse pancreas cancer cells (4B), treated in the presence or absence of ARS-1620. Shown in (C) are whole cell lysates and 12C-ARS Fab pull-downs (PD) from H358 and MIAPaCa-2 cells, treated as indicated. Shown in (D) is ARS-adduct formation in samples from C, quantified by LC/MS-MS assay. ARS-1620 and SHP099 concentrations were 10 μM in all panels.

FIG. 5. Data showing that 12C-ARS-Fab59 can be used to measure the engagement of ARS-1620 to mutant KRAS by pull-down assay with lysates prepared from animal tissues. A, B, anti-pan RAS and anti-ERK2 (loading control) immunoblots of lysates and 12C-ARS Fab pull-downs (PD) from LSL-KRAS^{G12C}-Tp53^{R270H} (A) and LSL-KRAS^{G12C} (B) tumors after 3 days of oral gavage with ARS-1620 (200 mg/kg/d) alone or with the SHP2 inhibitor SHP099 (75 mg/kg/d) are shown.

FIG. 6. Binding of antibody clones to AMG510 conjugated to the KRAS(G12C) peptide and the poly-Ser peptide. For each antibody clone, the bars are from left to right are No target, KRAS(G12C), KRAS(G12C)-AMG, and Unrelated peptide-AMG. Signals for the two negative controls, no target and the KRAS(G12C) peptide without a conjugated drug, were too low to be visible in the graph. The antibody clones were displayed on the yeast cell surface, and binding of the targets conjugated to fluorescently labeled streptavidin was detected using flow cytometry.

FIG. 7. Binding of the P2AMR-1 clone in the human IgG1 format to AMG510 conjugated to the KRAS(G12C) peptide (circles) and the same peptide without drug conjugation (squares). The peptide was immobilized to streptavidin-coated beads, and the antibody bound to the beads were detected with a fluorescently labeled secondary antibody. The apparent K_{D} value is shown. Data shown here are from triplicate measurements. Error bars are within the size of the symbols.

FIG. 8. Recognition of AMG510 presented on class I MHC molecule. The AMG510-RAS(G12C) conjugate (circles) and unconjugated peptide (squares) were loaded onto HLA-A*03:01 and immobilized on streptavidin-coated beads. Antibody binding was detected using the same method as in FIG. 2. The apparent K_{D} value is shown. Data shown here are from triplicate measurements. Error bars are within the size of the symbols.

FIG. 9. Cartoon representation of the disclosed concept referred to as HapImmune.

FIG. 10. Data representing development of antibodies that bind MHC/peptide-drug conjugate complexes. (A) Multiplex bead-binding assay (MBBA) of phages displaying different antibody clones. (B) MBBA assay of phages displaying different antibody clones to: HLA-A*01:01 in complex with the BTK peptide conjugated with Ibrutinib. (C) MBBA assay of phages displaying different antibody clones to: HLA-A*02:01 in complex with the EGFR peptide conjugated with Osimertinib.

FIG. 11. Binding titration graphs using the multiplex bead-binding assay (MBBA) of purified antibodies targeted to the KRAS(G12C)-AMG510 conjugate. Clone names are shown over each graph. Antigen nomenclature is described in FIG. 10.

FIG. 12. Graph showing that binding of antibodies to the AMG510-peptide conjugate in complex with an HLA was not affected by the presence of the free drug, AMG510.

FIG. 13. Graphs of binding titrations using the multiplex bead-binding assay (MBBA) of purified antibodies targeted to the BTK-Ibrutinib conjugate. Clone names are shown over each graph. Antigen nomenclature is described in FIG. 10.

FIG. 14. Graphs of binding titrations of purified antibodies to the KRAS(G12C)-AMG510 conjugate presented by endogenous HLA molecules on the cell surface. Raji cells were first incubated with the KRAS(G12C)-AMG510 conjugate or the KRAS(wild type) peptide, and excess conjugate and peptide were washed away. The antibody levels detected using a fluorescently labeled secondary antibody are shown as a function of IgG concentration used for staining. Apparent dissociation constant (K_{D}) values were determined using nonlinear least-squared fitting of a 1:1 binding function. Data shown here are from triplicate measurements.

FIG. 15. Antibody binding to a KRAS(G12C)-expressing cell line pretreated with AMG510. (A) Flow cytometry histograms. (B) Quantification of the median fluorescence intensity of H358 cells treated with or without AMG510. (C) Quantification of the median fluorescence intensity of H358 cells and HEK293T cells (a negative control) treated with or without AMG510 and stained with the AMRA3-7 antibody.

FIG. 16. Graph showing binding of P2AMR-1 IgG to cells preincubated with the KRAS(G12C) peptide-AMG510 conjugate, KRAS(wild type) peptide, or no peptide.

FIG. 17. Graphs showing binding of purified antibodies in the IgG format to the indicated drug-peptide/HLA complexes as measured using the multiplex bead binding assay (MBBA). (A) Antibody clones identified with AMG510 conjugated to KRAS(G12C) peptide in complex with HLA-A*03:01 as the antigen. (B) Antibody clones identified with AMG510 conjugated to KRAS(G12C) peptide in complex with HLA-*11:01 as the antigen.

FIG. 18. Graphs showing the cytotoxic effect of single-chain Diabodies (scDbs) on cells pulsed with an exogenous peptide-drug conjugate. (A) Raji cells were first pulsed with AMG510 conjugated to a peptide corresponding to a fragment of KRAS(G12C) or a control peptide corresponding to KRAS(wild type). The pulsed cells were co-cultured with human T cells (Effector:Target=3:1) in the presence of single-chain Diabodies (scDbs) at the indicated concentrations. After incubation, dead cells were stained and detected by flow cytometry. Data shown here are from triplicate measurements. Error bars indicate the s.d. Where error bars are not visible, the errors are smaller than the symbols. (B) Equivalent experiments using T2 cells and Osimertinib conjugated with an EGFR peptide. As a negative control, peptide conjugated with beta-mercaptoethanol was used.

FIG. 19. Graphs showing specific cytotoxic effect of AMRA3-7_UCHT1 scDb on drug-treated lung cancer cell lines. (A) Lung cancer cell lines were treated with 100 nM AMG510 for 24 hr, then co-cultured with human T-cells (E:T=5:1) in the presence of AMRA3-7_UCHT1 scDb. After incubation, cell viability was measured. The scDb antibody showed a dose-dependent cytotoxic effect only on the AMG510-treated cells with the cognate KRAS mutation (G12C) and HLA (HLA-A3). (B) Cytotoxic effect of scDb at 0.1 nM concentration. Data shown here are from quadruplicate measurements.

FIG. 20. Binding titration curves of AMR-A3-7 and AMR-A3-7D displayed on the yeast cell surface. Binding to HLA-A*03:01 presenting AMG510 conjugated to the Cys residue in the 9mer and 10mer RAS(G12C) peptides, VVGACGVGK and VVVGACGVGK, respectively, is shown.

FIG. 21. Graphs showing cell killing effects of AMRA3-7D scDb. (A) Dose-dependent cell killing effect of AMRA3-7D scDb tested with Raji cells that were first pulsed with AMG510 conjugated to a peptide corresponding to a fragment of KRAS(G12C) or a control peptide corresponding to KRAS(wild type). (B) Cell killing effect of AMRA3-7D scDb tested against H2122 non-small cell lung cancer cell line treated with AMG510 or DMSO only (negative control).

FIG. 22. Cell binding and killing effects of AMRA3-7D CrossMab. (A) Binding of AMRA3-7D to Jurkat cells, which express CD3 and to Raji cells, which do not express CD3. (B) Dose-dependent cell killing effect of AM1RA3-7D CrossMab tested with Raji cells that were first pulsed with AMG510 conjugated to a peptide corresponding to a fragment of KRAS(G12C) or a control peptide, corresponding to KRAS(wild type). (C) Cell killing effect of AMRA3-7D CrossMab tested with H2122 non-small cell lung cancer cell line treated with AMG510 or DMSO only (negative control).

FIG. 23. Deep mutational scanning of AMR-A3-7D. (a) Representative flow cytometry profiles of yeast cells displaying AMRA3-7D and its deep mutational scanning library populations. (b) The prevalence of mutations at each position in the sorted subsets of the deep mutational scanning library is shown in a heat map format. FIG. 23 discloses “GGWYPA” as SEQ ID NO: 155 and “ISYVKKLI” as SEQ ID NO:153.

FIG. 24. Deep mutational scanning of OEA2-5. (a) Representative flow cytometry profiles of yeast cells displaying OEA2-5 in the single-chain Fv format and its deep mutational scanning library populations. (b) The prevalence of mutations at each position in the sorted subsets of the deep mutational scanning library is shown in a heat map format. FIG. 24 discloses “EYVTMAL” SEQ ID NO: 159 and “YSYWPI” as SEQ ID NO: 157.

### DETAILED DESCRIPTION

Unless defined otherwise, all technical and scientific terms used herein have the same meaning as commonly understood by one of ordinary skill in the art to which this invention belongs.

Every numerical range given throughout this specification includes its upper and lower values, as well as every narrower numerical range that falls within it, as if such narrower numerical ranges were all expressly written herein.

As used in the specification and the appended claims, the singular forms “a” “and” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about” it will be understood that the particular value forms another embodiment. The term “about” in relation to a numerical value is optional and means for example +/−10%.

This disclosure includes every amino acid sequence described herein and all nucleotide sequences encoding the amino acid sequences. Every antibody sequence and antigen binding fragments of them are included. Polynucleotide and amino acid sequences having from 80-99% similarity, inclusive, and including and all numbers and ranges of numbers there between, with the sequences provided here are included in the invention. All of the amino acid sequences described herein can include amino acid substitutions, such as conservative substitutions, that do not adversely affect the function of the protein that comprises the amino acid sequences. In this regard, the disclosure provides alternative residues for certain positions in described binding partner as described below. In certain examples, the alternative residues were identified by deep mutational scanning, which demonstrates binding functionality for each binding partner that contains the described amino acid change(s). The disclosure includes each binding partner with each alternative residue substituted for the original residue alone and in any combination with the described alternative residues. Thus, any binding partner described herein may have any single described residue change or a combination of described changes. Representative changes for particular antibodies are described in Table A, Table B, Table C, and Table D. The changes may be in CDR1, CDR2, CDR3, and combinations thereof. The changes can also include amino acid insertions. The disclosure includes each amino acid sequence that is encompassed by the description of alternative amino acids by reference to a specific sequence identifier and those described in the aforementioned Tables.

As described above, the present disclosure provides antibodies and antigen binding fragments thereof (collectively “binding partners” and each individually a “binding partner”). The term “antibody” includes each binding partner format herein. The binding partners bind with specificity to a protein or fragment thereof, or a peptide provided in peptide form, that comprises a covalently attached molecule. The covalently attached molecule forms a peptide conjugate. A “peptide conjugate” as used herein means any protein or peptide that has been modified so that it is covalently conjugated to another molecule. The peptide conjugate is considered to be a novel antigen, i.e., a neoantigen. The other molecule that is covalently conjugated to the protein or peptide to form the peptide conjugate is not particularly limited, with the proviso that the other molecule is not an additional amino acid that is added to the described peptide conjugates. In embodiments, the molecule that is covalently conjugated to the protein or peptide has or had biological activity before conjugation, or it may be biologically inert before conjugation. In embodiments, the molecule is a drug, including but not necessarily limited to small molecule drugs. Representative and non-limiting examples of drugs that covalently attach to a peptide or protein to form a peptide conjugate are described below. Peptide conjugates include but are not limited to covalently modified full length proteins and fragments thereof. Peptide conjugates include fragments of full length proteins that include a covalent modification and are produced, for example, by intracellular processing. In certain embodiments, a full length protein may be covalently modified within a cell and subsequently processed such that a peptide conjugate that is a fragment of the full length protein is produced. As described further below, the produced peptide conjugate may be displayed on a cell surface. The cell surface display of the peptide conjugate may be any form of cell surface display, including but not limited to by way of any receptor having an extracellular segment, or it may be displayed by way of any type of major histocompatibility complex (MHC) or human leukocyte antigen (HLA). Non-limiting examples of HLA types that display peptide conjugates, and to which the described binding partners bind with specificity, are described further below.

In embodiments, the binding partners preferentially bind to the protein or peptide or a complex comprising the protein or peptide when covalently bound to the peptide conjugate, relative to the same protein or peptide that is not bound to the drug. Accordingly, binding partners described herein either do not detectably bind, or bind with a lower affinity, to the same protein or fragment thereof in the absence of the covalently attached molecule. In embodiments, the binding partners bind to the protein or peptide comprising the covalently attached drug with an affinity that is 10-10,000 fold, including all numbers and ranges of numbers from 10-10,000, greater than the affinity for the protein or peptide that does not comprise the covalently bound molecule. In this regard, and without intending to be bound by any particular theory, it is considered that the presence of the covalently bound molecule contributes to the presence of an epitope to which the binding partners bind with specificity.

In embodiments, the molecule that is covalently bound to form the peptide conjugate is a drug and may be any targeted covalent inhibitor (TCI), but an inhibition property is not necessarily required. In embodiments, the molecule reacts with a specific residue within the target protein. In embodiments, the molecule reacts at least in part with a segment of the protein or peptide that comprises a nucleophilic, or an electrophilic, residue. In embodiments, the segment of the protein or peptide to which the molecule reacts comprises any of Cys, Lys, Tyr, and His. In embodiments, the molecule reacts at least in part with a segment of the protein or peptide that comprises a wild type Cys, or a mutation of a residue to a Cys, and thus may be covalently attached by a so-called sulfur tether. In embodiments, the drug is any drug described in Ghosh A K, Samanta I, Mondal A, Liu W R. Covalent Inhibition in Drug Discovery. ChemMedChem. 2019; 14(9):889-906. doi:10.1002/cmdc.201900107, or in De Cesco, et al., European Journal of Medicinal Chemistry 138 (2017) 96e114, or in Bauer, R A, Drug Discovery Today, Volume 20, Number 9, September 2015, from which the disclosures of compounds that covalently modify protein targets is incorporated herein by reference.

In non-limiting embodiments, any of said Cys, Lys, Tyr, and His amino acids are present in the protein or peptide to which the molecule binds because the wild type protein has been mutated to include one or a combination of the described residues. In non-limiting embodiments, the molecule binds to a protein or peptide that is correlated with a condition, such as a cancer. In embodiments, the target (e.g., the protein or peptide to which the molecule covalently binds) is a receptor, including but not necessarily limited to any receptor having a catalytically active segment. In embodiments, the drug binds to an enzyme that is not necessarily a receptor, including but not limited to any kinase. In embodiments, a protein target comprises a receptor with one or more activating mutations, which promote ligand-independent enzyme activity.

In embodiments, the molecule targets and thus covalently binds to an amino acid sequence present within any of the following proteins and/or variants thereof, which may or may not comprise a mutation, such as a mutation that is related to a particular condition, including but not limited to any type of cancer. In embodiments, the protein is any protein described in Visscher M, et al., Covalent targeting of acquired cysteines in cancer. Curr Opin Chem Biol. 2016; 30:61-67. doi:10.1016/j.cbpa.2015.11.004, from which the description is incorporated herein by reference. Visscher et al. also teaches methods for identifying disease-associated mutated genes that introduces a Cys residue suitable for covalent modification. In embodiments, the protein is KRAS, Bruton's tyrosine kinase (BTK), any member of the epidermal growth factor receptor (EGFR) family, also referred to as the ERBB family, including but not limited to EGFR (ERBB1), HER2/NEU (ERBB2), HER3 (ERBB3), and HER4 (ERBB4); a fibroblast growth factor receptor (FGFR); the receptor kinase known in the art as MET, BRAF, a cyclin-dependent kinase (CDK); Acetyl Choline Esterase (ACHE); TP53, IDH1, GNAS, FBXW7, CTNNB1, DNMT3A, any cathepsin, including cathepsin B, C, F, H, K, L, O, S, V, W and X; any caspase; any protein involved in obesity, such as Pancreatic lipase and METAP2, or any Cancer Testis Antigen. In embodiments, the drug targets and therefore covalently binds to any viral protein, including but not limited to a polymerase, including any viral DNA polymerase, RNA polymerase, reverse transcriptase, or RNA-dependent RNA polymerase, or a viral protein that is required, for example, viral cell entry, or a protein encoded by any a transposable element. In embodiments, the drug targets EGFR and may be selected from PD168393, PF00299804 (dacomitinib), EKB569 (pelitinib), afatinib, WZ4002, osimertinib (formerly known as AZD9291), PF-06459988, nazartinib, naquotinib, olmutinib, avitinib, and rociletinib, neratinib, pyrotinib, poziotinib, and derivatives thereof. In embodiments, the drug targets Bruton's tyrosine kinase (BTK), and may be selected from ibrutinib, acalabrutinib, zanubrutinib, CHMFL-BTK-11, ONO/GS-405, PRN1008, and CC-292. In embodiments, the drug targets any p90 ribosomal S6 kinase (RSK), and may be selected from fluoromethylketone (FMK) and dimethyl fumarate. In embodiments, the drug targets any FGFR, and may be selected from FIIN-1, FIIN-2, FIIN-3, BGJ398, AZD4547, PRN1371, FGF401.

In a non-limiting embodiment, the binding partner binds with specificity to a site comprised by a neoantigen that includes a covalently linked small molecule drug or other covalently linked molecule as a component of an antigen in a specific MHC context.

In embodiments, the molecule that becomes covalently bound to form the peptide conjugate targets any RAS oncogene protein product, including but not necessarily limited to HRAS, NRAS, KRAS4A, and KRAS4B. The amino acid sequences of RAS proteins are known in the art, and residue numbering is identical for the relevant part of all RAS isotypes that are discussed in this disclosure for which the amino acid sequence is available from, for example, UniProt P01116, from which the amino acid sequence is incorporated herein as of the effective filing date of this application or patent. The G12 position is numbered according to the known amino acid sequence, regardless of whether or not the G12 is the twelfth amino acid in an express RAS peptide sequence of this disclosure.

In one embodiment, the molecule covalently binds to a KRAS protein or peptide that comprises a mutation. In embodiments, the mutation is at least one of KRAS residues 12, 13, or 61. Reference to any drug herein includes its name in capitalized and un-capitalized form.

In embodiments, the drug targets a KRAS protein comprising a KRAS G12C mutation. In non-limiting embodiments, the drug that targets a KRAS protein is selected from 2E07, 6H05, SML-8-73-1, MRTX849, JNJ74699157, LY3499446, ARS-853, ARS-1620, MRTX1275, AMG510, or derivatives thereof. In an embodiment the drug comprises a proteolysis targeting chimera (PROTAC) derivative of a covalent drug, a non-limiting description of which is available in doi: 10.1021/acscentsci.0c00411, from which the description of PROTACs is incorporated herein by reference. In embodiments, the PROTAC is LC-1 or LC-2. In embodiments, the disclosure relates to an autophagy-mediated degrader, referred to as an AUTAC, as described in doi.org/10.1080/15548627.2020.1718362, from which the description of AUTACs is incorporated herein by reference.

A non-limiting example of a binding partner that binds to KRAS(G12C)-AMG510 is referred to herein as AMRA3-7D. The disclosure includes all derivatives of AMRA3-7D that are described herein, including the alternative residues described below by way of deep mutational analysis, and in the forms of an scDb and a CrossMab, for which representative amino acid sequences are provided. The amino acid sequence of the light chain (VL) and heavy chain (VH) of AMRA3-7D are:

V_{L}:
(SEQ ID NO: 3)
DIQMTQSPSSLSASVGDRVTITCRASQSVSSAVAWYQQKPGKAPKLLIY
SASSLYSGVPSRFSGSRSGTDFTLTISSLQPEDFATYYCQQISYVKKLI
TFGQGTKVEIKRTV
V_{H}:
(SEQ ID NO: 4)
EVQLVESGGGLVQPGGSLRLSCAASGFTFSDYSIHWVRQAPGKGLEWVA
SISSSSGSTSYADSVKGRFTISADTSKNTAYLQMNSLRAEDTAVYYCAR
GGWYPAMDYWGQGTLVTVSS

A non-limiting example of a binding partner that binds to an Epidermal Growth Factor receptor (EGFR)-osimertinib conjugate is referred to herein as OEA2-5. The disclosure includes all derivatives of OEA2-5 that are described herein, including the alternative residues described below by way of deep mutational analysis, and in the form of an scDb, for which representative amino acid sequences are provided.

The amino acid sequences of the light chain (VL) and heavy chain (VH) of OEA2-5 are:

V_{L}:
(SEQ ID NO: 5)
DIQMTQSPSSLSASVGDRVTITCRASQSVSSAVAWYQQKPGKAPKLLIY
SASSLYSGVPSRFSGSRSGTDFTLTISSLQPEDFATYYCQQYSYWPITF
GQGTKVEIKRTV
V_{H}:
(SEQ ID NO: 6)
EVQLVESGGGLVQPGGSLRLSCAASGFTISSSYIHWVRQAPGKGLEWVA
YISPSYGSTSYADSVKGRFTISADTSKNTAYLQMNSLRAEDTAVYYCAR
EYVTMALDYWGQGTLVTVSS

In embodiments, the binding partner binds to a protein in its native form, with the exception that the drug or other molecule is covalently attached to it. “Native form” means the intact protein that retains its biological function before covalent attachment of the drug or other molecule. In embodiments, the native form or the protein is its form before being fragmented such as by intracellular processing. In embodiments, the binding proteins therefore bind to full length polypeptides that are covalently attached to the drug or other molecule and wherein the covalently bound drug or other molecule at least in part permits the preferential binding of the binding partners. In general, a polypeptide, which is used interchangeably herein with the term “protein,” comprises more than 50 contiguous amino acids. In embodiments, a binding partner binds with specificity to an intact protein that is covalently attached to a drug or other molecule. In other embodiments, the binding partners bind with specificity to a peptide comprising the covalently bound molecule. In embodiments, the binding partner binds with specificity to a peptide having a specific amino acid sequence and is covalently conjugated to another molecule, such as a drug. In embodiments, the binding partner binds preferentially to a peptide covalently bound to a molecule such as a drug, where the sequence of the peptide is not relevant. This preferential binding is relative to binding to the same peptide that is not conjugated to the drug. In embodiments, the binding partner binds preferentially to a peptide comprising a KRAS(G12) mutation, or to a variant thereof, wherein the variant is at least 50% similar to the KRAS(G12)-containing peptide. This preferential binding is relative to binding to a KRAS(G12)-containing peptide, or the variant thereof, respectively, that is not covalently conjugated to the drug or other molecule.

In embodiments, the described binding partners bind with specificity to peptide conjugates that are of suitable length to be presented in a major histocompatibility complex (MHC), referred to as human leukocyte antigen (HLA) in humans, or to MHC or its equivalent complex in non-human animals, including but not limited to non-human mammals.

In general, the peptide conjugate comprises fewer than 50 contiguous amino acids. In embodiments, peptide conjugates which comprise the described epitope may therefore be from 2-49 amino acids in length. In embodiments, the peptide to which the drug or other molecule is covalently attached, and which attached drug may be comprised by the epitope, comprises from 4-12 contiguous amino acids, which may or may not be derived from a longer protein during the processing of a protein, such as an antigen processed for presentation by an MHC molecule. In embodiments, the drug is conjugated to a peptide that comprises, or consists of 7-30 amino acids. In embodiments, the drug or other molecule is conjugated to a peptide that comprises, or consists of, 7, 8, 9, 10, 11, 12, 13, 14, or 15 amino acids, and which may be presented in an MHC Class I context. In embodiments, the drug or other molecule is conjugated to a peptide that is 9-30 amino acids, inclusive, and including all numbers and ranges of numbers there between, and which may be presented in an MHC Class II context. In embodiments, the drug or other molecule is conjugated to a peptide comprises at least 7 amino acids.

In embodiments, a binding partner binds with specificity to a peptide conjugate that is covalently conjugated to a drug or other molecule independent of MHC presentation. In embodiments, non-limiting examples of which are described below in Example 3, the binding partner binds with specificity to the peptide conjugate only when the peptide conjugate is presented by an MHC molecule. In embodiments, the binding partner can bind with specificity to a peptide conjugate in both an MHC-independent and an MHC-presentation context. In embodiments, the MHC-peptide conjugate complex comprises an antigen to which a described binding partner binds with specificity.

In embodiments, the binding partners accordingly can bind to cells via any MHC that can present peptide conjugates. In embodiments, the HLA is expressed by cells that are restricted to Class I, Class II, or Class III MHC presentation. In embodiments, the binding partners can bind to cells that express Class I MHC that presents the peptide conjugate. Those skilled in the art will recognize that Class I MHC includes, among other components, a polymorphic α chain and β2 microglobulin, wherein the peptide conjugate binds to the polymorphic chain.

In embodiments, the cells are antigen presenting cells (APCs). In embodiments, the cells are so-called professional antigen presenting cells, and thus may include but are not limited to macrophages and dendritic cells, which display Class II MHC. Those skilled in the art will recognize that Class II MHC includes, among other components, MHC polymorphic α and β chains, and the displayed peptide conjugate binds to both chains. In other embodiments, Class II MHC may be displayed with the peptide conjugate by other cell types, such as cancer/tumor cells, and thus the disclosure provides for direct recognition of such cells using the described binding partners, without requirement for a professional APC.

In embodiments, the peptide conjugate is displayed by a non-classical MHC complex, which may include CD1d, MR1, MHC-E, -F, -G and/or other emerging family members that will be recognized by those skilled in the art.

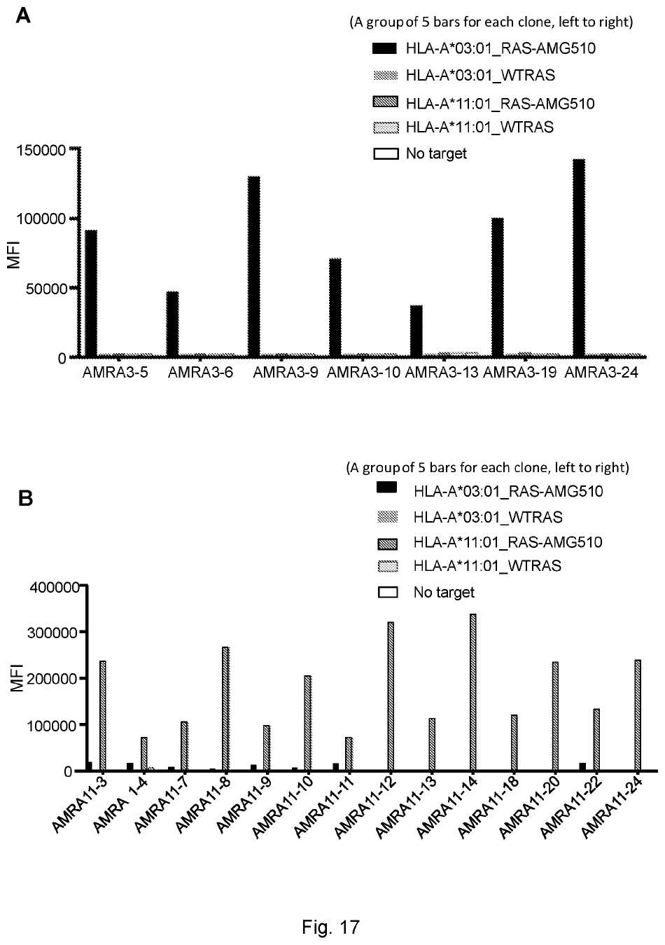

The disclosure includes binding partners that bind with specificity to a peptide conjugate displayed only by a specific MHC type, and thus provides binding partners that discriminate between MHC types. Representative examples of such binding partners are described herein at least by way of FIG. 17.

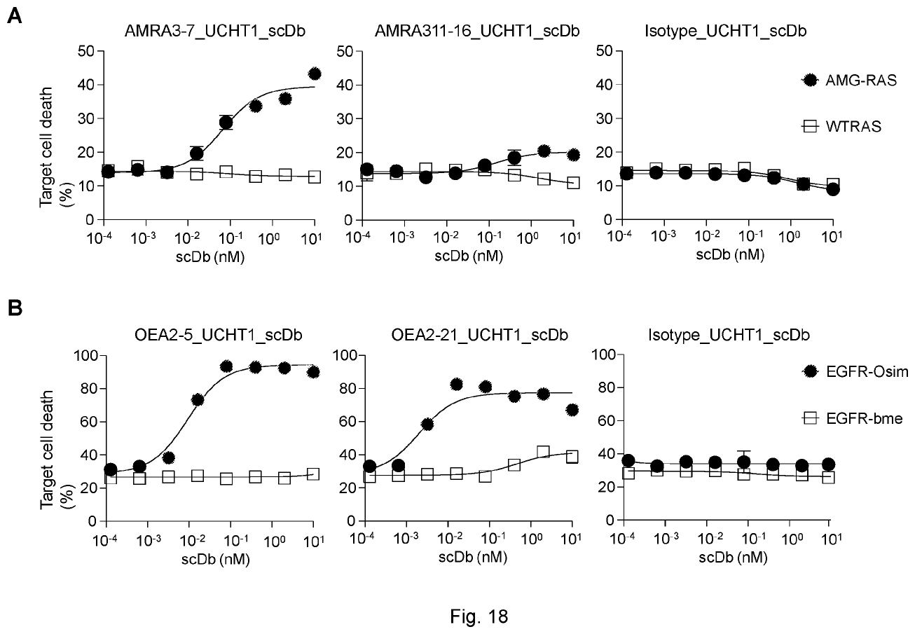

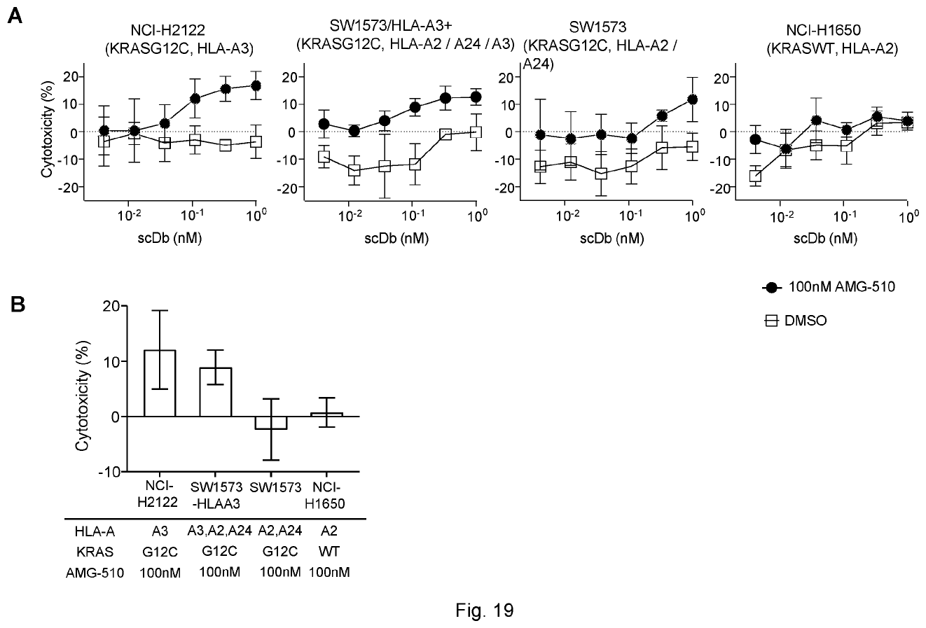

In embodiments, a binding partner of this disclosure can bind with specificity to a peptide conjugate comprising a covalently conjugated drug or other molecule that is displayed by more than one specific MHC type. In embodiments, a binding partner of this disclosure can bind with specificity to a peptide conjugate comprising a covalently conjugated drug only in a specific MHC context. In embodiments, the peptide conjugate is displayed by an MHC class I type selected from HLA-A, -B, -C, and combinations thereof. In certain aspects, the peptide conjugate is displayed in the context of any MHC class I that is A*02/B*35/C*04. In embodiments, the peptide conjugate is displayed by any of MHC of class II that is DR*01/DR*04/DR*07/DP*04. In embodiments, the HLA comprises A*01:01, A*02:01, A*03:01, A*11:01, A*24:02, A*26:01, B*07:02, B*08:01, B*27:05, B*39:01, B*40:01, B*58:01, or B*15:01. Specific examples of antibodies include antibodies that bind to KRAS(G12C)-AMG510 conjugate presented on HLA-A*03:01 and HLA-A*11:01, BTK-Ibrutinib conjugate presented on HLA-A*01:01, and EGFR-Osimertinib conjugate presented on HLA-A*02:01. In non-limiting embodiments, the disclosure provides scDbs that are specific for a particular drug that is covalently bound to a described peptide that is present on a specific HLA, or the same drug that is covalently bound to a described peptide that is present on two different HLAs. Representative scDbs are described in Example 4. Data obtained using the scDbs are presented via FIGS. 18 and 19. Data obtained using CrossMab formats are provided in Example 5 and its accompanying figures.

In embodiments, the peptide conjugate is displayed by cells that participate in, or can be the targets of, cell-mediated immune responses. In embodiments the peptide conjugate that is displayed in any suitable MHC context is comprised by a cell that is recognized by a leukocyte, including but not necessarily limited to a T cell or a natural killer (NK) cell. In embodiments, the T cell is a CD4+ T cell, a CD8+ T cell, a double positive CD4+/CD8+ T cell, a CD4+/CD8+ double negative T cell, or a γδ T cell. Thus, and as described further below, the disclosure provides binding partners that are configured to interact with both the presented peptide conjugate and cells that participate in cell-mediated immune responses. In embodiments, certain described binding partners are capable of binding to a complex of 1) a specific MHC and 2) a specific peptide conjugate. In embodiments, certain described binding partners are capable of being bound to a specific peptide conjugate presented by at least two different MHCs.

In embodiments, any binding partner of this disclosure comprises at least one chain that comprises a complementary determining region (CDR) that is CDR1, CDR2, or CDR3 from any heavy or light chain amino acid sequence described herein. In certain examples in the present specification, the CDRs are shown in bold font. The amino acid sequences of the CDR sequences are separately encompassed by this disclosure by way of their positions in the described heavy and light chain amino acid sequences. The disclosure includes binding partners that comprise a described heavy chain CDR1, CDR2, and CDR3. The disclosure also includes binding partners that comprise a described light chain CDR1, CDR2, and CDR3. The disclosure also includes binding partners that comprise a described heavy chain CDR1, CDR2, and CDR3 and a described light chain CDR1, CDR2, and CDR3. For amino acid sequences of this disclosure that include amino acids that comprise purification or protein production tags, such as HIS tags and/or AVI-tags, the disclosure includes the proviso that the sequences of the described tags may be excluded from the amino acid sequences. Amino acids between the described tags may also be excluded.

Binding partners of this disclosure can be provided as intact immunoglobulins or as fragments of immunoglobulins, including but not necessarily limited to antigen-binding (Fab) fragments, Fab′ fragments, (Fab′)₂ fragments, Fd (N-terminal part of the heavy chain) fragments, Fv fragments (two variable domains), diabodies (Dbs), dAb fragments, single domain fragments or single monomeric variable antibody domains, single-chain Diabodies (scDbs), isolated complementary determining regions (CDRs), single-chain variable fragment (scFv), and other antibody fragments that retain antigen binding function. In embodiments, one or more binding partners are provided as a component of a Bi-specific T-cell engager (BiTE), bispecific killer cell engager (BiKE), CrossMab (e.g., a binding partner containing four different chains; immunoglobulin crossover (also known as Fab domain exchange or CrossMab format) technology (see eg., WO2009/080253; Schaefer et al., Proc. Natl. Acad. Sci. USA, 108:11187-11192 (2011).), or a chimeric antigen receptor (CAR), such as for producing chimeric antigen receptor T cells (e.g., CAR T cells) and CAR natural killer (NK) cells, and killer macrophages. The disclosure includes binding partners that include the described heavy and light chain variable regions.

In embodiments, the binding partners are multivalent. In embodiments, a tri-specific binding partner is provided. In embodiments, cells express at least a segment of one or more binding partners in the form of a CAR. In an embodiment, a binding partner of this disclosure may be provided as a complex with a polynucleotide, such as an RNA polynucleotide, to form an aptamer. In embodiments, a multi-valent binding partner includes one binding component, such as a paratope, that confers specificity to a particular target on a desired cell type, such as any cancer cell marker. In embodiments, a tri-specific leukocyte engager is provided. In embodiments, the binding partners may be part of a molecule that is activated only in the presence of a protease or other enzyme present in a tumor microenvironment, such embodiments being pertinent to, for instance, a probody, examples of which are known in the art, for example in doi: 10.1126/scitranslmed.3006682, doi: 10.1038/s41467-020-16838-w, and doi: 10.1038/s41587-019-0135-x, from which the descriptions of probodies, and protease activation, are incorporated herein by reference. In an embodiment, the disclosure provides a universal hapten that can be grafted onto inhibitors.

In embodiments, a CAR of this disclosure comprises scFv that comprises heavy and light chains as described herein. As is known in the art for previously described CARs, the scFv is present in a contiguous polypeptide that further comprises a CD3zeta chain and a costimulatory domain. In embodiments, the costimulatory domain comprises a 4-1BB costimulatory domain or a CD28 costimulatory domain. A CAR may also contain a co-receptor hinge sequence, such as a CD8 a co-receptor hinge sequence.

In embodiments, binding partners of this disclosure may comprise a constant region, e.g., an Fc region. Any isotype of constant region can be included. Binding partners that comprise a constant region may be particularly adapted for antibody-dependent cell mediated cytotoxicity (ADCC) and thus may function to kill targeted cells by cell-mediated responses by any of a variety of effector cells. Similarly, a constant region may be particularly adapted for enhancing complement-mediated responses.

In embodiments, a binding partner of this disclosure may be modified such that it is present in a fusion protein. In embodiments, an antigen binding segment of a binding partner may be present in a fusion protein, and/or the constant region may be a component of a fusion protein. In embodiments, a fusion protein comprises amino acids from at least two different proteins. Fusion proteins can be produced using any of a wide variety of standard molecular biology approaches, including but not necessarily limited to expression from any suitable expression vector. In embodiments, a binding partner described herein may be present in a fusion protein with a detectable protein, such as green fluorescent protein (GFP), enhanced GFP (eGFP), mCherry, and the like. In embodiments, as an alternative to an expression vector, an mRNA or chemically modified mRNA encoding any binding partner described herein can be delivered to cells such that the binding partner is translated by the cells.

In embodiments, binding partners described herein are used to carry drugs or toxins, and thus the binding partners may be provided as immunotoxins, or in the form of antibody-drug conjugates (ADCs).

In embodiments, agents useful in the generation of immunotoxins include enzymatically active toxins and enzymatically active fragments thereof. Suitable enzymatically active toxins include but are not limited to diphtheria A chain, nonbinding active fragments of diphtheria toxin, exotoxin A chain (from Pseudomonas aeruginosa), ricin A chain, abrin A chain, modeccin A chain, alpha sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. These can be provided as components of fusion proteins or can be covalently attached to the binding partner by any suitable conjugation approach.

The binding partner may be connected to a chemotherapeutic agent by using any suitable linker to form an antibody drug conjugate (ADC). In embodiments, the linker comprises a disulfide, a hydrazine, or a thioether. The chemotherapeutic agent may be reversibly or irreversibly attached to the binding partner.

Cleavable linkers may be particularly useful for killing bystander cells. In embodiments, a protease recognition site may be included to liberate the chemotherapeutic agent from the binding partner by operation of a protease that recognizes and cleaves at the protease recognition site. The ADC may therefore be considered to contain a prodrug.

In embodiments, binding partners of this disclosure may comprise linking sequences. As a non-limiting example, an ScFv may comprise a linker that links segments comprising paratopes to one another. Suitable amino acid linkers may be mainly composed of relatively small, neutral amino acids, such as glycine, serine, and alanine, and can include multiple copies of a sequence enriched in glycine and serine. In specific and non-limiting embodiments, the linker comprises 3, 4, 5, 6, 7, 8, 9, 10, 11, 12, 13, 14, 15, 16, 17, 18, 19, and 20 amino acids. In an example, the linker may be the glycine-serine-alanine linker G₄SA₃ (SEQ ID NO: 11) or a glycine-serine linker (G₄S)₄ linker (SEQ ID NO: 12). In embodiments, a binding partner may include a cellular localization signal, or a secretion signal. In embodiments, binding partner may comprise a transmembrane domain, and thus may be trafficked to, and anchored in a cell membrane. For secretion, any suitable secretion signal can be used, and many are known in the art.

In embodiments, the binding partners can be part of an ADC and therefore the binding partners comprise a drug. The drug can include, but is not necessarily limited to, any suitable chemotherapeutic agent. In embodiments, the ADC comprises a binding partner and a chemotherapeutic agent that is an anti-microtubule agent, an alkylating agent, or a DNA minor groove binding agent. In embodiments, the chemotherapeutic agent comprises a maytansinoid, a dolastatin, an auristatin drug analog, or a cryptophycin. In embodiments, the chemotherapeutic agent is a duocarmycin derivative, or an antibiotic, such as an enediyne antibiotic, or pyrolobenodiazepine (PBD), including dimers thereof. In embodiments, the chemotherapeutic agent is an enzyme inhibitor, such as a topoisomerase or polymerase inhibitor. In embodiments, the chemotherapeutic agent comprises doxorubicin, or a metal-containing compound, such as a platinum-containing compound, non-limiting examples of which include cisplatin, carboplatin or oxaliplatin. In embodiments, the ADC comprises a binding partner described herein, and any drug that is described in Barf and Kaptein, dx.doi.org/10.1021/jm3003203, J. Med. Chem. 2012, 55, 6243-6262, or in Wilson et al., dx.doi.org/10.1021/jm400224q, J. Med. Chem. 2013, 56, 7463-7476, or Lambert and Morris, Adv Ther (2017) 34:1015-1035, from which the descriptions of drugs for use as components as ADCs is incorporated herein by reference. In embodiments, the binding partner is conjugated to or otherwise includes a cytokine, including but not necessarily limited to an interleukin, including but not limited to IL-2 and IL-12, or an interferon (IFN), to thereby provide a cytokine conjugate.

For production of binding partners, any suitable expression system may be used. In general, polynucleotides encoding binding partners are used to express the binding partners in any suitable cell system, non-limiting embodiments of which include NS0 murine myeloma cells, human cell lines, and Chinese hamster ovary (CHO) cells. In embodiments, the disclosure provides a polynucleotide that can selectively hybridize to a polynucleotide encoding any CDR or combination of CDRs described herein. In embodiments, the polynucleotide selectively hybridizes to a polynucleotide encoding a heavy chain CDR1, CDR2, and CDR3 of any described binding partner. In embodiments, the polynucleotide selectively hybridizes to a polynucleotide encoding a light chain CDR1, CDR2, and CDR3 of any described binding partner. In embodiments, the polynucleotide selectively hybridizes to a polynucleotide encoding CDR1, CDR2, and CDR3 of a heavy and light chain of any described binding partner.

In embodiments, a binding partner described herein may be a component of a fusion protein. In embodiments, such as for a binding partner that is produced as a fusion protein, a peptide linker may be used. In embodiments, the peptide linker comprises any self-cleaving signal. In embodiments, the self-cleaving signal may be present in the same open reading frame (ORF) as the ORF that encodes the binding partner. A self-cleaving amino acid sequence is typically about 18-22 amino acids long. Any suitable sequence can be used, non-limiting examples of which include: T2A (EGRGSLLTCGDVEENPGP (SEQ ID NO: 7)); P2A (ATNFSLKQAGDVENPGP (SEQ ID NO: 8)); E2A (QCTNYALKLAGDVESNPGP (SEQ ID NO: 9)) and F2A (VKQTLNFDLKLAGDVESNPGP (SEQ ID NO: 10)).

To the extent any segment of a protein comprising a binding partner described herein was a component of a library, including but not necessarily limited to a phage display library or a yeast surface display library, the disclosure includes the proviso that the binding partner may be free of any segment of the library that comprises a bacteriophage or yeast amino acid sequence, including but not limited to phage coat protein or a yeast host protein, including but not limited to Aga2. Thus, in certain embodiments, the binding partner may be present in a fusion protein, but the fusion protein does not comprise bacteriophage coat protein. In embodiments, any binding partner described herein may be free of any of pIII phage coat protein, or any part of Ml, fd filamentous phage, T4, T7, or λ phage protein.

In embodiments, a binding partner of this disclosure comprises a detectable label, which may be used for diagnostic or therapeutic purposes. For example, a detectable label can be used for localization of the binding partner for pathology and/or in vivo imaging approaches. In embodiments, a binding partner is conjugated to any of a variety of radioactive agents, including but not limited to a highly radioactive atom, such as In111, At211, I131, I125, Y90, Re186, Re188, Sm153, Bi212, P32, Pb212, and radioactive isotopes of Lu. In particular embodiments, such as for imaging, the binding partner may be conjugated to a radioactive atom for scintigraphic approaches, for example Tc99m (metastable technetium-99), 1123, or a spin label for nuclear magnetic resonance (NMR) imaging (also known as magnetic resonance imaging, or “MRI”), such as 1123, 1131, 1124, F19, C13, N15, O17 or Gadlinium (III) or Manganese (II). In embodiments, the radioactive agent is suitable for use in CAT scan or PET imaging. In embodiments, Indium111, Technetium99 or Iodine131 can be used for planar scans or single photon emission computed tomography (SPECT). Positron emitting labels such as Fluorine19 Iodine 123 and Iodine 124 can be used in positron emission tomography. Paramagnetic ions such as Gadlinium (III) or Manganese (II) can used in magnetic resonance imaging MRI. In embodiments, the described radioactive isotopes that are attached to a described binding partner can also be used in therapeutic approaches. In embodiments, radioactive agents or isotopes include alpha-emitting radionuclides. In embodiments, radioactive agents or isotopes include beta-emitting radionuclides. In some embodiments, the present disclosure provides an antibody of the present technology conjugated to a diagnostic or therapeutic agent. The diagnostic agent may comprise a radioactive or non-radioactive label, a contrast agent (such as for magnetic resonance imaging, computed tomography or ultrasound), and the radioactive label can be a gamma-, beta-, alpha-, Auger electron-, or positron-emitting isotope. A diagnostic agent is a molecule which is administered conjugated to an antibody moiety, i.e., antibody or antibody fragment, or subfragment, and is useful in diagnosing or detecting a disease by locating the cells containing the antigen.

Any binding partner described herein may be fully or partially humanized. Techniques for humanization of antibodies are known in the art and can be adapted for use in the present disclosure. In embodiments, humanization may be performed, for example, by CDR-grafting. In embodiments, for humanization or to otherwise improve a characteristic of the binding partners, one or more amino acids in a variable region can be changed. In embodiments, one or more amino acids in a framework region can be changed.

The disclosure includes binding partners for use in diagnostic and therapeutic approaches. For therapeutic approaches, in certain embodiments, binding partners may be delivered as mRNA or DNA polynucleotides that encode the binding partners. It is considered that administering a DNA or RNA encoding any binding partner described herein is also a method of delivering such binding partners to an individual or one or more cells. Methods of delivering DNA and RNAs encoding proteins are known in the art and can be adapted to deliver the binding partners, given the benefit of the present disclosure. In embodiments, one or more expression vectors are used and comprise viral vectors. Thus, in embodiments, a viral expression vector is used. Viral expression vectors may be used as naked polynucleotides, or may comprise any of viral particles, including but not limited to defective interfering particles or other replication defective viral constructs, and virus-like particles. In embodiments, the expression vector comprises a modified viral polynucleotide, such as from an adenovirus, a herpesvirus, or a retrovirus. In embodiments, a retroviral vector adapted from a murine Moloney leukemia virus (MLV) or a lentiviral vector may be used, such as a lentiviral vector adapted from human immunodeficiency virus type 1 (HIV-1).

In an embodiment, an oncolytic viral vector is used. Oncolytic viruses (OVs), including vaccinia (OVV), mediate anticancer effects by both direct oncolysis and stimulation of innate immune responses through production of damage-associated molecular patterns (DAMPs) and the presence of virus-derived pathogen-associated molecular patterns (PAMPs), leading to increased type I interferon production. Additionally, OVV-mediated oncolysis may facilitate the direct acquisition of tumor-derived antigens by host antigen-presenting cells within the tumor microenvironment, thereby leading to improved T cell priming as well as coordination of the effector phase of antitumor immune responses. In alternative embodiments, a recombinant adeno-associated virus (AAV) vector may be used. In certain embodiments, the expression vector is a self-complementary adeno-associated virus (scAAV).

Pharmaceutical formulations containing binding partners are included in the disclosure and can be prepared by mixing them with one or more pharmaceutically acceptable carriers. Pharmaceutically acceptable carriers include solvents, dispersion media, isotonic agents, and the like. The carrier can be liquid, semi-solid, e.g. pastes, or solid carriers. Examples of carriers include water, saline solutions or other buffers (such as phosphate, citrate buffers), oil, alcohol, proteins (such as serum albumin, gelatin), carbohydrates (such as monosaccharides, disaccharides, and other carbohydrates including glucose, sucrose, trehalose, mannose, mannitol, sorbitol or dextrins), gel, lipids, liposomes, resins, porous matrices, binders, fillers, coatings, stabilizers, preservatives, liposomes, antioxidants, chelating agents such as EDTA, salt forming counter-ions such as sodium; non-ionic surfactants such as TWEEN, PLURONICS or polyethylene glycol (PEG), or combinations thereof. In embodiments, a liposomal formulation comprising one or more binding partners is provided. Liposomal formulations include but are not limited to liposomal nanoparticles.

In embodiments, an effective amount of one or more binding partners is administered to an individual in need thereof. In embodiments, an effective amount is an amount that reduces one or more signs or symptoms of a disease and/or reduces the severity of the disease. An effective amount may also inhibit or prevent the onset of a disease or a disease relapse. A precise dosage can be selected by the individual physician in view of the patient to be treated. Dosage and administration can be adjusted to provide sufficient levels of binding partner to maintain the desired effect. Additional factors that may be taken into account include the severity and type of the disease state, age, weight, and gender of the patient, desired duration of treatment, method of administration, time and frequency of administration, drug combination(s), reaction sensitivities, and/or tolerance/response to therapy.

Binding partners and pharmaceutical compositions comprising the binding partners can be administered to an individual in need thereof using any suitable route, examples of which include intravenous, intramuscular, intraperitoneal, intracerobrospinal, subcutaneous, intra-articular, intrasynovial, oral, topical, or inhalation routes, depending on the particular condition being treated. The compositions may be administered parenterally or enterically. The compositions may be introduced as a single administration or as multiple administrations or may be introduced in a continuous manner over a period of time. For example, the administration(s) can be a pre-specified number of administrations or daily, weekly, or monthly administrations, which may be continuous or intermittent, as may be therapeutically indicated.

In embodiments, the individual in need of a composition of this disclosure has been diagnosed with or is suspected of having cancer. In embodiments, the cancer is a solid tumor or a hematologic malignancy. In embodiments, the cancer is renal cell carcinoma, breast cancer, prostate cancer, pancreatic cancer, lung cancer, liver cancer, ovarian cancer, cervical cancer, colon cancer, esophageal cancer, glioma, glioblastoma or another brain cancer, stomach cancer, bladder cancer, testicular cancer, head and neck cancer, melanoma or another skin cancer, any sarcoma, including but not limited to fibrosarcoma, angiosarcoma, osteosarcoma, and rhabdomyosarcoma, and any blood cancer, including all types of leukemia, lymphoma, and myeloma. In embodiments, the individual is in need of treatment for any pre-neoplastic disorder, including myelodysplastic syndromes or myeloproliferative neoplasms. In embodiments, a described binding partner is used prophylactically for any of the described types of cancer.

In embodiments, administering one or more binding partners, including but not necessarily in a pharmaceutical formulation, to an individual in need thereof, exhibits an improved activity relative to a control. In an embodiment, the control comprises different antibodies, a different form of the same antibodies/binding partner, or antibodies/binding partners that are delivered without adding additional agents. In embodiments, a binding partner described herein provides for improved antibody dependent cell cytotoxicity (ADCC), or for internalization (such as for an ADC), relative to a control. In embodiments, a control protein or peptide does not comprise the covalently linked molecule. The control peptide may comprise the same sequence as the experimental peptide, or if the experimental peptide comprises a mutation the control peptide may comprise the wild type sequence.

A composition of this disclosure, such as a pharmaceutical formulation, can contain only one, or more than one binding partner, and thus combinations of different binding partners are included. Likewise, one or more binding partners can be combined with any other therapeutic agent, non-limiting examples of which include conventional chemotherapeutic agents, and modulators of T-cell costimulatory molecules, often referred to as immune checkpoint inhibitors. T-cell costimulatory molecules are known in the art (PMID 30115704), including, but not limited to, CTLA4, PD-1, PD-L1, LAG3, TIM3, TIGIT, VISTA, B7-1, B7-2, PD-L2, LSECtin, Galectin-9, CEACAM-1, CD155, CD112, CD28, ICOS, ICOSL, OX40, OX40L, GITR, GITRL, 4-1BB, 4-1BBL, CD40, CD40L, CD27, and CD70. Thus, the disclosure includes combination therapy using one or more described binding partners and any of modulators of T-cell costimulatory molecules, including but not limited to CTLA-4 inhibitors, PD-1 inhibitors and PD-L1 inhibitors. As non-limiting examples, anti-PD-1 agents include Pembrolizumab and Nivolumab. Anti-PD-L1 examples include Avelumab and Atezolizumab. An anti-CTLA-4 example is Ipilimumab. The binding partners may also be combined with any form of adoptive immunotherapy.

In embodiments, the disclosure comprises administering to an individual in need thereof one or more binding partners and at least one additional agent to provide an additive effect, or a greater than additive effect such as a synergistic result. In embodiments, the described effect comprises inhibition of cancer growth, inhibition of metastasis, or other beneficial effect. An additive effect or synergistic effect may also be achieved by using a combination of at least two described binding partners.

Various techniques have been developed for the production of binding partners and are included in the scope of this disclosure. In embodiments, the binding partners are produced by host cells by way of recombinant expression vectors. The present disclosure includes all polynucleotide sequences encoding the amino acid sequences described herein, expression vectors comprising such polynucleotide sequences, and in vitro cell cultures comprising such expression vectors. In embodiments, the cell cultures include prokaryotic cells or eukaryotic cells. In embodiments, the cell cultures are mammalian cells. In embodiments, the cells are CHO cells. In embodiments, the cells are HEK293 cells and their derivatives. Kits comprising the binding partners, and/or cell cultures expressing the binding partners, are provided by this disclosure. In general, the kits comprise one or more sealed containers that contain the binding partners, or cells expressing them. Instructions for using the binding partners for therapeutic and/or diagnostic purposes can be included in the kits.

Cells that are modified to express any described binding partner include but are not necessarily limited CD4+ T cells, CD8+ T cells, Natural Killer T cells, γδ T cells, and cells that are progenitors of T cells, such as hematopoietic stem cells or other lymphoid progenitor cells, such as immature thymocytes (double-negative CD4−CD8−) cells, or double-positive thymocytes (CD4+CD8+). In embodiments, the progenitor cells comprise markers, such as CD34, CD117 (c-kit) and CD90 (Thy-1). In embodiments, the modified cells comprise macrophages. The described modified cells may be used therapeutically or prophylactically.

In embodiments, the disclosure provides for generation of a binding partner. This approach comprises providing a plurality of distinct binding partners, exposing the plurality of distinct (e.g., different) binding partners to one or a diversity of peptide conjugates, and selecting binding partners that bind with specificity to the peptide conjugates that contain the covalently conjugated drug or other molecule, but do not bind to the protein or peptide that does not comprise the covalently conjugated drug or other molecule. As described above, this approach can be performed on a manner that either does, or does not, require the amino acid sequence of the protein or peptide to be part of the antigenic determinant. The described approach can be used to select binding partners that are specific for presentation of a peptide conjugate as a component of any MHC complex.

In embodiments, binding partners described herein and as otherwise will be apparent by those skilled in the art, can be used to determine whether or not a particular drug or other molecule forms a covalent interaction with a protein or peptide. Thus, the disclosure provides for exposing protein or peptide substrates to drug candidates and using the binding partners described herein or as identified as described herein to determine whether or not the drug forms a covalent interaction with the pertinent substrate. This determination can be made based on whether or not the binding partner binds to the protein or peptide that has been covalently attached to the drug. This approach can be used in lieu of currently available techniques, such as mass spectroscopy and the like.

In embodiments, binding partners of this disclosure may be used in any immunological diagnostic test, including but not limited to the imaging approaches described above. In embodiments, one or more binding partners described herein can be used as a component in any form of, for example, enzyme-linked immunosorbent assay (ELISA) assay, including but not limited to a direct ELISA, a sandwich ELISA, a competitive ELISA, and a reverse ELISA. In embodiments, one or more binding partners described herein can also be incorporated into an immunodiagnostic device, such as a microfluidic device, a lateral flow device, and the like. The binding partners may also be used in, for example, Western blots and immunoprecipitation assays.

The following Examples are intended to illustrate but not limit the disclosure. In embodiments, antibodies described in Example 3 have different properties relative to those described in Example 1. Other differences between binding partners will be apparent from the Examples and their accompanying figures. The different properties include, but are not necessarily limited to, specificity for a drug conjugate displayed in the context of a specific MHC type. Thus, binding partners may exhibit different binding partners when a peptide conjugates is in a particular MHC complex.

### Example 1

This Example provides a description of the identification and characterization of binding partners that bind with specificity to ARS-1620, which forms a covalent interaction with KRAS^{G12C}.

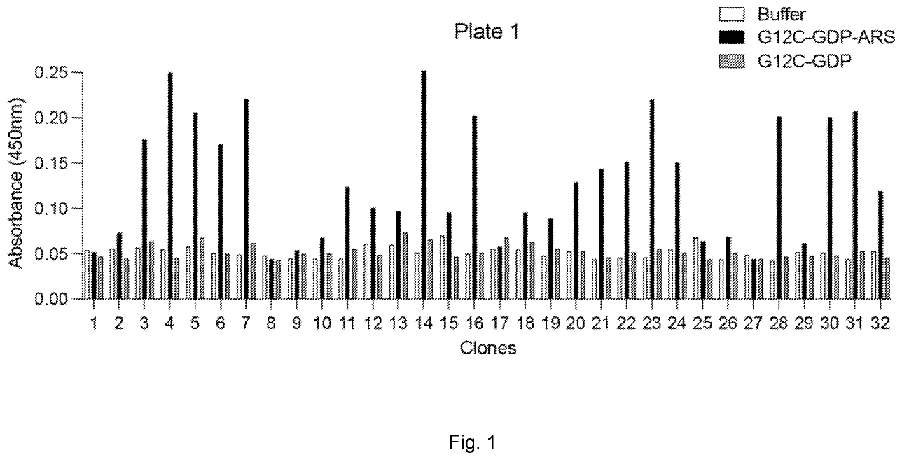

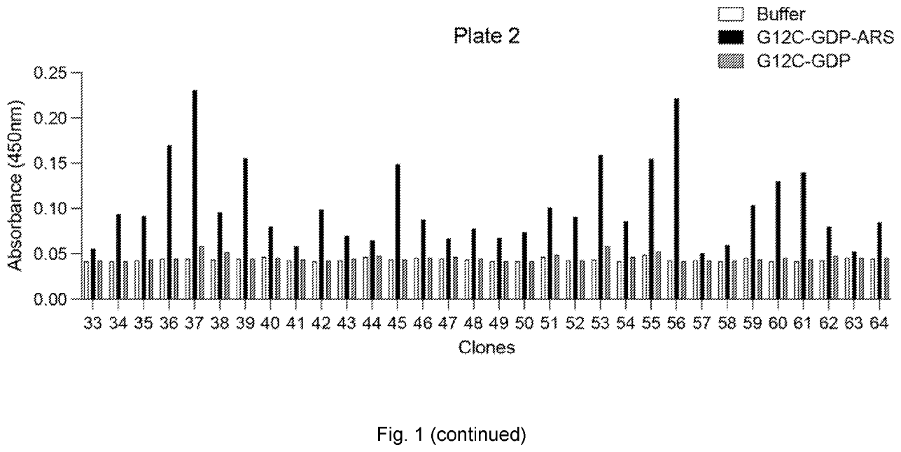

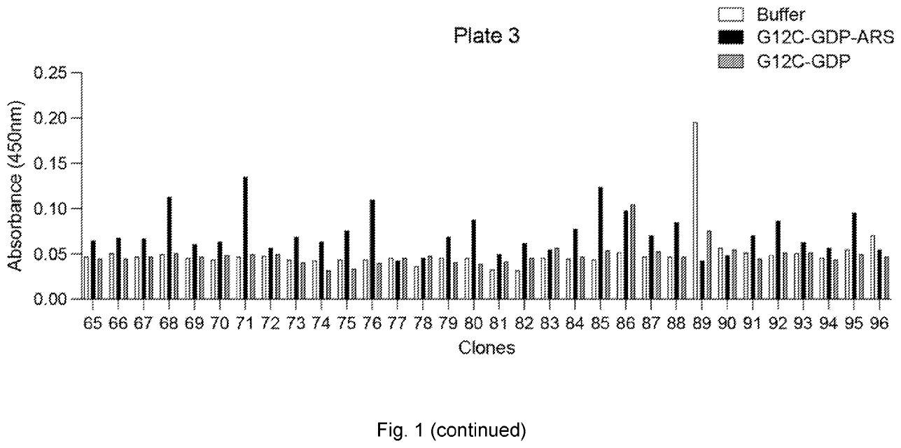

In particular, FIG. 1 demonstrates phage ELISA of phage-displayed antibody clones.

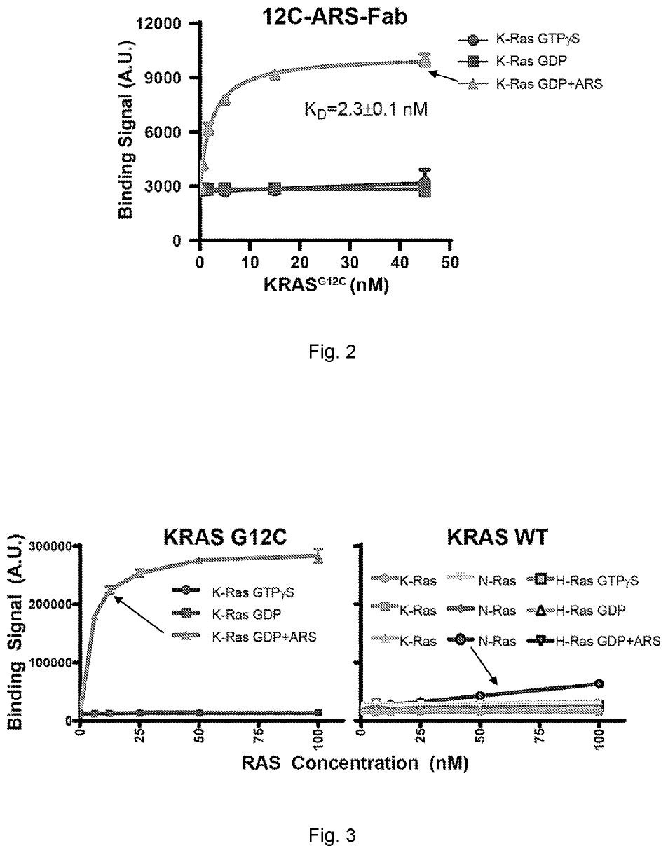

Binding to KRAS^{G12C}-GDP and KRAS^{G12C}-GDP-ARS-1620 conjugate was determined. Buffer denotes binding signal to the wells that did not contain KRAS^{G12C}. From these candidates, four different antibodies were identified. Among these, 12C-ARS-Fab59 showed high affinity binding to KRASG12C-GDP covalently bound to ARS-1620. The results are presented in FIG. 2, which shows 12C-ARS Fab59 binding to KRAS^{G12C} in the GTPγS- or GDP-bound nucleotide state with or without ARS-1620, as characterized by the bead binding assay (PMID: 33358997). FIG. 3 also demonstrates that 12C-ARS-Fab59 specifically binds KRAS^{G12C}-GDP conjugated to ARS-1620. In particular, 12C-ARS-Fab binding to KRAS^{G12C} (left) or WT RAS isoforms (right) in the GTPγS- or GDP-bound nucleotide state with or without ARS-1620 conjugation, is shown as indicated.

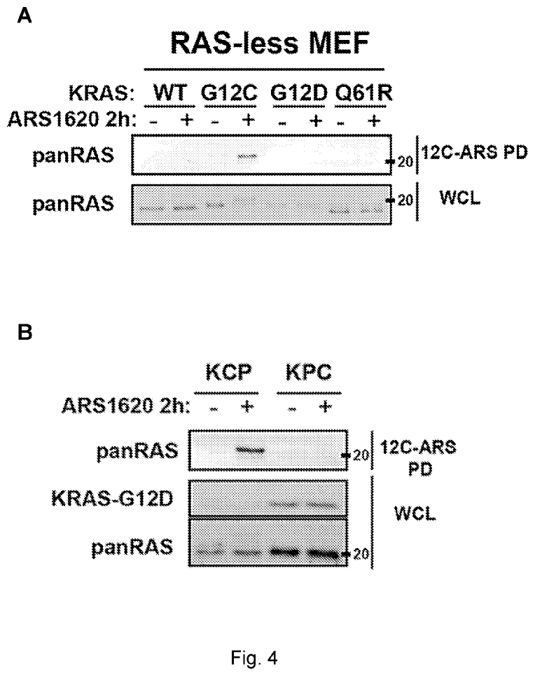

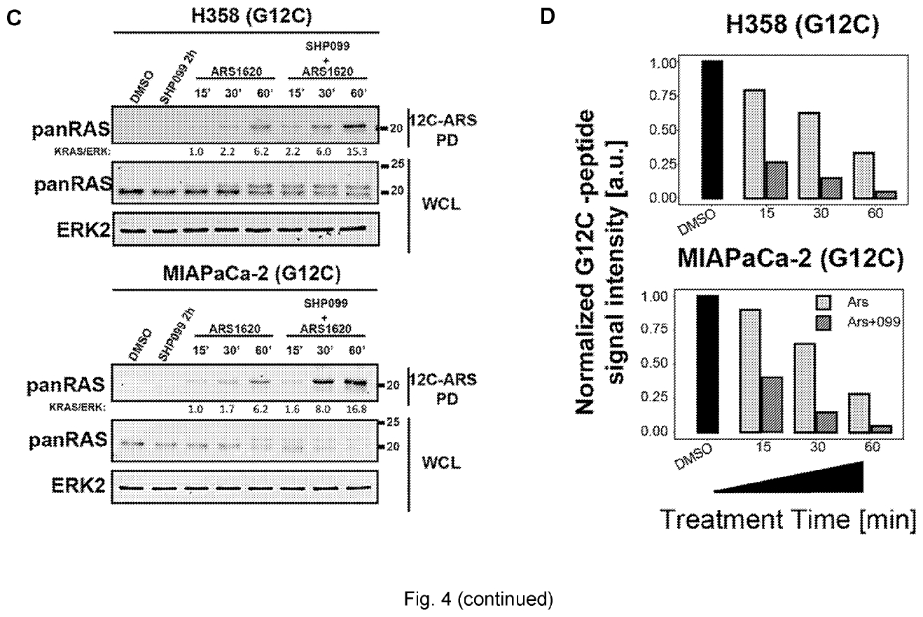

FIG. 4 demonstrates the use of 12C-ARS-Fab59 to measure ARS-1620/KRAS^{G12C} adducts by pull-down assays from lysates prepared from cell lines. To produce the data shown in FIG. 4, immunoblots were performed on whole cell lysates and 12C-ARS Fab-pull-downs (PD) from RAS-less MEFs reconstituted with the indicated KRAS mutants (4A) and from KCP (Kras^{G12C}; TP53^{R172H}; Pdx-Cre) mouse pancreas cancer cells (4B), treated in the presence or absence of ARS-1620. FIG. 4C shows whole cell lysates and 12C-ARS Fab pull-downs (PD) from H358 and MIAPaCa-2 cells, treated as indicated, which were subjected to SDS-PAGE and immunoblotting with anti-pan RAS and anti-ERK2 antibodies, the latter as a loading control. FIG. 4D shows ARS-adduct formation in samples from 4C, quantified by LC/MS-MS assay. ARS-1620 and SHP099 concentrations were 10 μM in all panels.

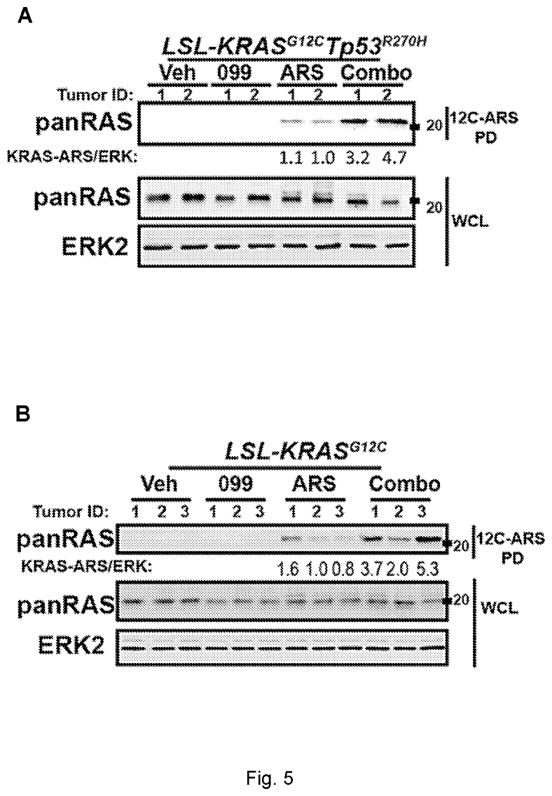

FIG. 5 shows that 12C-ARS-Fab59 can be used to measure the engagement of ARS-1620 to mutant KRAS by pull-down assay with lysates prepared from animal tissues. In particular, in FIGS. 5A-B, anti-pan RAS and anti-ERK2 (loading control) immunoblots of lysates and 12C-ARS Fab pull-downs (PD) from LSL-KRAS^{G12C}-Tp53^{R270H} (A) and LSL-KRAS^{G12C} (B) tumors after 3 days of oral gavage with ARS-1620 (200 mg/kg/d) alone or with the SHP2 inhibitor SHP099 (75 mg/kg/d) are shown.

To produce the foregoing results, the following materials and methods were used.

### RAS Nucleotide Exchange and Generation of ARS-1620-Conjugated RAS

Purified RAS (1-174) proteins containing a 6×HIS-tag (SEQ ID NO: 13) and an AVI-tag (1), used in the binding experiments and phage display selections, were prepared by diluting stock protein (typically containing 20-100 μM RAS) 25-fold with 20 mM Tris-Cl buffer pH 7.5 containing 5 mM EDTA, 0.1 mM DTT, and 1 mM (final concentration) of nucleotide (GDP or GTPγS). For generating ARS-bound RAS, ARS-1620 (final concentration: 100 μM) was added during the nucleotide exchange reaction of RAS along with GDP. Samples were incubated at 30° C. for 30 minutes. MgCl₂ was then added to a final concentration of 20 mM to quench the nucleotide exchange reaction, and the solution was incubated on ice for at least 5 minutes prior to use.

### Selection of Phage-Displayed Antibody Fragments Against ARS-Bound KRAS^{G12C}

General procedures for the development of Fabs against purified protein targets have been described (2). Four rounds of phage display library selection were performed, with biotinylated KRAS(G12C)-GDP+ARS-1620 at 100 nM, 100 nM, 50 nM, and 20 nM in the first, second, third and fourth rounds, respectively. The first round recovered clones that bound to KRAS^{G12C}-GDP+ARS-1620; the second round recovered clones that bound to KRAS^{G12C}-GDP+ARS-1620, previously pre-cleared with KRAS^{G12C}-GDP; the third round recovered clones that bound to KRAS^{G12C}-GDP+ARS-1620, previously pre-cleared with KRAS^{G12C}-GTP. The final round recovered clones that bound to KRAS^{G12C}-GDP+ARS-1620, previously precleared with KRAS^{G12C}-GDP. Phage captured on beads were eluted in 100 μl of 0.1 M Gly-HCl (pH 2.1) and immediately neutralized with 35 μl of 1M Tris-Cl (pH 8). Recovered clones were analyzed by phage ELISA and DNA sequencing, as described (2).

### Bead Binding Assays

General methods have been previously described (3). Fifty microliters (50 μl) of M280 streptavidin beads (Thermo Fisher) were incubated with 100 μl of biotinylated 12C-ARS Fab, at 30 nM or 4 nM. Ligand-free streptavidin on the beads was then blocked by adding excess biotin. Beads were washed with supplemented TBST (50 mM Tris pH7.5, 150 mM NaCl, 20 mM MgCl₂, 0.1 mM DTT, 0.05% Tween-20) and dispensed into wells of a 96-well U bottom plate (Greiner). Beads were then incubated at 1:1 ratio with purified RAS proteins diluted in supplemented TBS (50 mM Tris pH7.5, 150 mM NaCl, 20 mM MgCl₂, 0.1 mM DTT) at 2× the concentration stated for the titration curve for 30 minutes at room temperature. Beads containing bound Fab and RAS were transferred to the wells of a 96-well filter plate (Millipore, MSHVN4550) and washed twice with supplemented TBST before incubating with Neutravidin-Dylight 650 (Thermo Fisher Scientific) for 30 minutes at 4° C. The beads were washed twice with supplemented TBST before resuspension in supplemented TBS for flow cytometry using an iQue screener (Sartorius). The median signal intensity in the Dylight650 channel for the 75-95th percentile population was taken as binding signal to the target. K_{D} was calculated by fitting the binding signals to a 1:1 binding model.

### Expression, Purification, and Characterization of Recombinant Fabs

Phage display vectors were converted into Fab expression vectors that contain a substrate tag for the biotin ligase BirA at the carboxyl terminus of the heavy chain. Fabs were expressed in E. coli strain 55244 (ATCC), and were purified by protein G affinity chromatography, followed by cation exchange chromatography, as described (2). Purified Fabs were biotinylated in vitro using purified BirA. Approximately 2-5 mg of purified Fabs were obtained routinely from a 1 L bacterial culture. SDS-PAGE showed that Fabs were >90% pure.

### KRAS^{G12C}-Adduct Assays

Cells cultured in 6-well plates were treated with ARS-1620 and/or SHP099 as described in the Figures. Cells were lysed by incubation in GTPase lysis buffer (25 mM Tris-Cl pH7.2, 150 mM NaCl, 5 mM MgCl2, 1% NP-40 and 5% glycerol), supplemented with protease inhibitors and phosphatase inhibitors on ice for 15 minutes immediately before analysis. After centrifugation for 15 minutes at 15,000 g, supernatants were collected and incubated with streptavidin (SA) agarose resin (Thermo Fisher Scientific) for 1 hour at 4° C., followed by a brief centrifugation, to decrease non-specific binding to the resin. Pre-cleared lysates were incubated with biotinylated 12C-ARS-Fab bound to SA agarose for 1.5 hours at 4° C. while rotating. Agarose beads were then washed twice with GTPase lysis buffer, boiled in 1×SDS-PAGE sample buffer, and subjected to immunoblotting with a pan-RAS antibody (Millipore).

### Immunoblotting

Whole cell lysates were generated in modified radioimmunoprecipitation (RIPA) buffer (50 mM Tris-HCl pH 8.0, 150 mM NaCl, 2 mM EDTA, 1% NP-40, and 0.1% SDS, without sodium deoxycholate), supplemented with protease (40 μg/ml PMSF, 2 μg/ml antipain, 2 μg/ml pepstatin A, 20 μg/ml leupeptin, and 20 μg/ml aprotinin) and phosphatase (10 mM NaF, 1 mM Na₃VO₄, 10 mM β-glycerophosphate, and 10 mM sodium pyrophosphate) inhibitors. After clarification of debris by centrifugation in a microfuge, samples were quantified with the DC Protein Assay Kit (Bio-Rad). Total lysate protein was resolved by standard SDS-PAGE and transferred in IX transfer buffer and 15% methanol. Membranes were incubated with their respective primary and secondary antibodies labeled with IRDye (680 nm and 800 nm) and then visualized by using a LICOR device. Monoclonal pan-RAS antibody (clone Ab-3; OP40-100UG; 1:1000) was obtained from Millipore, and mouse monoclonal ERK-2 (D2: sc-1647; 1:1000) was purchased from Santa Cruz Biotechnology.

### LC/MS-MS Assay for ARS Binding to KRAS^{G12C}

Cells (5×10⁵) were treated with the indicated compounds for the times listed and subsequently washed twice with PBS and prepared for protein extraction and LC/MS-MS analysis, as described (4). LC/MS-MS was performed at the PCC Proteomics Shared Resource at NYU School of Medicine.

Similar methods were used to obtain the results described in Example 2.

The antibodies described in this Example are as follows:

### Exemplary Antibody Clones Binding to the KRAS(G12C)-ARS-1620 Conjugate:

CDR residues (Kabat numbering) in bold.

12C-ARS-Fab59
V_{L}:
(SEQ ID NO: 14)
DIQMTQSPSSLSASVGDRVTITCRASQSVSSAVAWYQQKPGKAPKLLIY
SASSLYSGVPSRFSGSRSGTDFTLTISSLQPEDFATYYCQQDWYFPITF
GQGTKVEIK
V_{H}:
(SEQ ID NO: 15)
EVQLVESGGGLVQPGGSLRLSCAASGFTFSSYYIHWVRQAPGKGLEWVA
SISPSSGSTYYADSVKGRFTISADTSKNTAYLQMNSLRAEDTAVYYCAR
YGGRSYWQKQDSYFYQHGLDYWGQGTLVSS
12C-ARS-Fab56
V_{L}:
(SEQ ID NO: 16)
DIQMTQSPSSLSASVGDRVTITCRASQSVSSAVAWYQQKPGKAPKLLIY
SASSLYSGVPSRFSGSRSGTDFTLTISSLQPEDFATYYCQQSSSSLITF
GQGTKVEIK
V_{H}:
(SEQ ID NO: 17)
EVQLVESGGGLVQPGGSLRLSCAASGFTFSSSSIHWVRQAPGKGLEWVA
SISSYSGYTSYADSVKGRFTISADTSKNTAYLQMNSLRAEDTAVYYCAR
SYSYSEFRYYYSGQGMDYWGQGTLVSS
12C-ARS-Fab30
V_{L}:
(SEQ ID NO: 18)
DIQMTQSPSSLSASVGDRVTITCRASQSVSSAVAWYQQKPGKAPKLLIY
SASSLYSGVPSRFSGSRSGTDFTLTISSLQPEDFATYYCQQSSSSLITF
GQGTKVEIK
V_{H}:
(SEQ ID NO: 19)
EVQLVESGGGLVQPGGSLRLSCAASGFTFSSSSIHWVRQAPGKGLEWVA
SISSSSGSTSYADSVKGRFTISADTSKNTAYLQMNSLRAEDTAVYYCAR
SNYGWRWHLVGMDYWGQGTLVTVSS
12C-ARS-Fab85
V_{L}:
(SEQ ID NO: 20)
DIQMTQSPSSLSASVGDRVTITCRASQSVSSAVAWYQQKPGKAPKLLIY
SASSLYSGVPSRFSGSRSGTDFTLTISSLQPEDFATYYCQQSSSSLITF
GQGTKVEIK
V_{H}:
(SEQ ID NO: 21)
EVQLVESGGGLVQPGGSLRLSCAASGFTFSSSSIHWVRQAPGKGLEWVA
SISSSSGSTSYADSVKGRFTISADTSKNTAYLQMNSLRAEDTAVYYCAR
SPYVYYWYMVGFDYWGQGTLVTVSS

This reference listing pertains to Example 1.
1. Spencer-Smith R, Koide A, Zhou Y, Eguchi R R, Sha F, Gajwani P, Santana D, Gupta A, Jacobs M, Herrero-Garcia E, Cobbert J, Lavoie H, Smith M, Rajakulendran T, Dowdell E, Okur M N, Dementieva I, Sicheri F, Therrien M, Hancock J F, Ikura M, Koide S, O'Bryan J P. Inhibition of RAS function through targeting an allosteric regulatory site. Nat Chem Biol. 2017; 13(1):62-8. doi: 10.1038/nchembio.2231. PubMed PMID: 27820802; PMCID: 5193369.
2. Fellouse F A, Esaki K, Birtalan S, Raptis D, Cancasci V J, Koide A, Jhurani P, Vasser M, Wiesmann C, Kossiakoff A A, Koide S, Sidhu S S. High-throughput generation of synthetic antibodies from highly functional minimalist phage-displayed libraries. J Mol Biol. 2007; 373(4):924-40. Epub 2007 Sep. 11. doi: 10.1016/j.jmb.2007.08.005. PubMed PMID: 17825836.
3. Nishikori S, Hattori T, Fuchs S M, Yasui N, Wojcik J, Koide A, Strahl B D, Koide S. Broad ranges of affinity and specificity of anti-histone antibodies revealed by a quantitative Peptide immunoprecipitation assay. J Mol Biol. 2012; 424(5):391-9. Epub 2012/10/09. doi: 10.1016/j.jmb.2012.09.022. PubMed PMID: 23041298; PMCID: 3502729.
4. Patricelli M P, Janes M R, Li L S, Hansen R, Peters U, Kessler L V, Chen Y, Kucharski J M, Feng J, Ely T, Chen J H, Firdaus S J, Babbar A, Ren P, Liu Y. Selective Inhibition of Oncogenic KRAS Output with Small Molecules Targeting the Inactive State. Cancer Discov. 2016; 6(3):316-29. Epub 2016 Jan. 8. doi: 10.1158/2159-8290.Cd-15-1105. PubMed PMID: 26739882.
5. doi.org/10.1084/jem.20201414

### Example 2

This Example provides a description of binding partners that bind with specificity to AMG510 that is covalently linked to peptides.

To produce the results described in this Example, some methods as described in Example 1 were adapted. For this Example, AMG510 (purchased from Selleckchem) was conjugated to a peptide corresponding to KRAS(G12C) residues 4-18:

(SEQ ID NO: 22)
H2N-YKLVVVGACGVGKSA(dPEG4)(K-long chain Biotin)-
amide

and a poly-Ser peptide containing a central Cys:

(SEQ ID NO: 23)
H2N-SSSSCSSSSW(K-long chain Biotin)-amide

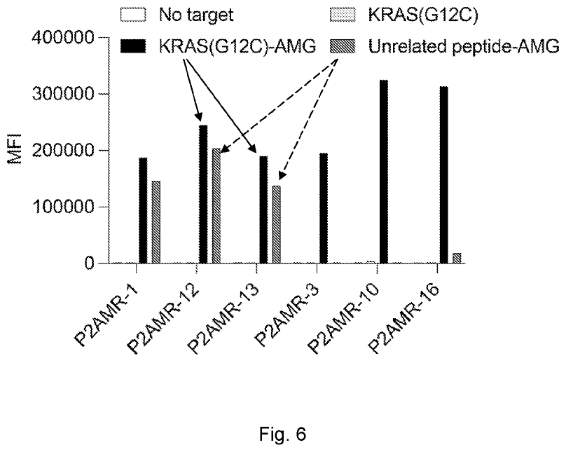

A human single-chain Fv yeast-display library was sorted using these peptides as targets by using established methods⁽¹⁻³⁾. After rounds of library sorting, individual clones were screened. We developed three antibodies that bound to AMG510 conjugated to both KRAS(G12C) and poly-Ser peptide (FIG. 6). Consequently, these antibodies recognize predominantly the AMG510 moiety but not the peptide moiety of the conjugates. Additionally, we developed other clones that are selective to AMG510 conjugated to the KRAS(G12C) peptide.

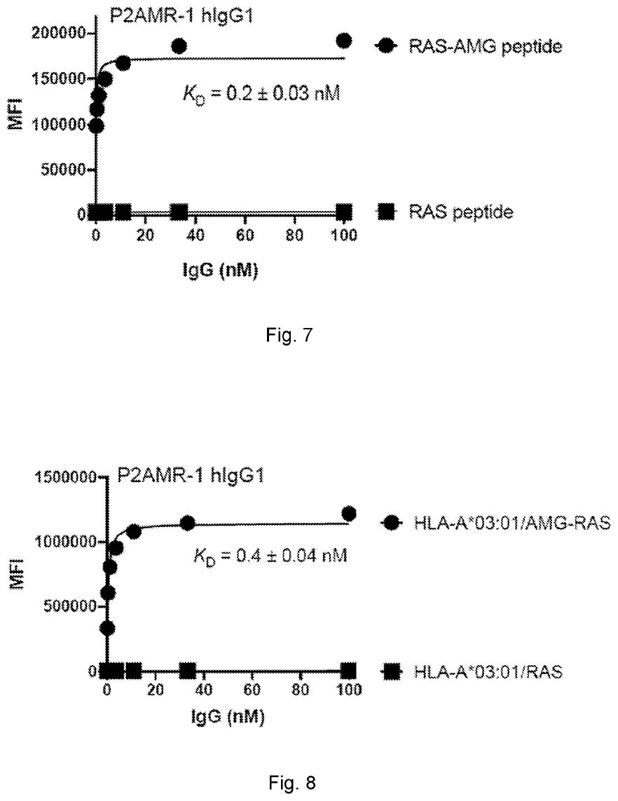

One such clone, P2AMR-1 was then produced in the format of human IgG1 and further characterized. It bound to AMG510 conjugated to the KRAS(G12C) peptide with high apparent affinity in a bead binding assay (FIG. 7)). The antibody clone also bound tightly to AMG510 conjugated a shorter KRAS(G12C) peptide, VVGACGVGK (SEQ ID NO: 1), in the context of HLA-A*03:01 (BioLegend Flex-T) (FIG. 8).

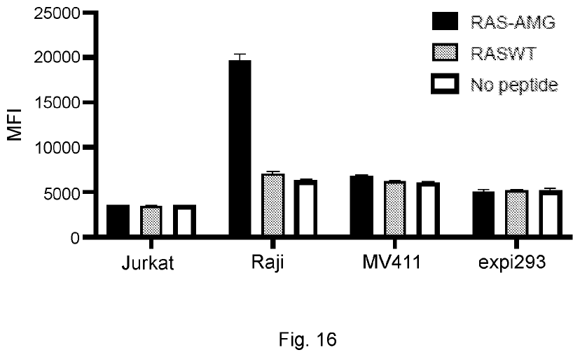

P2AMR-1 detected AMG510 conjugated to KRAS(G12C) peptide that had been added to Raji cells, which are known to express HLA-A*03:01. By contrast, P2AMR-1 did not detect KRAS(wild type) peptide loaded in the same manner (FIG. 16). In addition, P2AMR-1 did not bind to AMG510 conjugated to the KRAS(G12C) peptide added to cells that are not known to express HLA-A*03:01, e.g., MV4-11 and Expi293 cells (FIG. 16).

These results demonstrate that that the presently provided antibodies, which represent binding partners of this disclosure, recognize the AMG510 moiety in a manner agnostic of the conjugation partner, and they suggest that our antibodies and their derivatives can be used to identify cells that present AMG510-KRAS(G12C) peptide conjugate on MHC molecules on the cell surface.

More generally, these results suggest methods for targeting any cells that harbor intracellular targets that form covalent adducts with small molecule ligands.

This example demonstrates the following non-limiting binding partners, restricted to AMG510 covalent modifications of the described substrates. CDR residues (Kabat scheme) are shown in bold.

P2AMR-1
V_{L}:
(SEQ ID NO: 24)
QSVLIQPRSVSGSPGQSVTISCTGTSSDVGGYNYVSWYQQHPGKAPKLM
IYDVSKRPSGVPDRFSGSKSGNTASLTVSGLQAEDEADYYCGSYADTDT
IVFGTGTKLTVL
V_{H}:
(SEQ ID NO: 25)
QVQLVQSEPEVKKPGSSVKLSCKASGGTFSTDAITWVRQAPGQGLEYMG
GIIPLLDSVDYAQRFQGRVTVSADKSTGTAYMEVRSLGSEDTAKYYCAK
WSSVDTGLDYWGQGTLVTVSS
P2AMR-12 (this clone has only the heavy chain)
V_{H}:
(SEQ ID NO: 26)
QVQLQESGPGLVKPSETLSLTCTVSGDSIINDPHYWGWIRQSPGKGLEW
IGSTSHSGHTYFNPSLKSRVSMSIDVAKNQFSLNVRSVTAADTAVYYCA
RMRYYYSGTYPVYYFDYWGQGTLVTVSS
P2AMR-13
V_{L}:
(SEQ ID NO: 27)
SYVLTQPPSASGTPGQRVTISCSGSSSNIGSNFVSWYQQLPGTAPKLLI
SSNNQRPSGVPDRFSGSKSDTSASLAISGLQSEDEADYYCAAWDDSLNG
PVFGGGTQLTVL
V_{H}:
(SEQ ID NO: 28)
QVQLVQSEAEVKKPGSSVKVSCKASGGTFSRYGVSWVRQAPGQGLEWMG
GIIPMFGTANYAQKFQGRVTITADESTSTAYMELRSLRSEDTAVYYCAR
GDNSAYSDAFNIWGQGTMVTVSS

This reference listing pertains to Example 2.
1. Chao G, Lau W L, Hackel B J, Sazinsky S L, Lippow S M, Wittrup K D. Isolating and engineering human antibodies using yeast surface display. Nat Protoc. 2006; 1(2):755-68. Epub 2007/04/05. doi: 10.1038/nprot.2006.94. PubMed PMID: 17406305.
2. Feldhaus M J, Siegel R W, Opresko L K, Coleman J R, Feldhaus J M, Yeung Y A, Cochran J R, Heinzelman P, Colby D, Swers J, Graff C, Wiley H S, Wittrup K D. Flow-cytometric isolation of human antibodies from a nonimmune Saccharomyces cerevisiae surface display library. Nat Biotechnol. 2003; 21(2):163-70. Epub 2003 Jan. 22. doi: 10.1038/nbt785nbt785 [pii]. PubMed PMID: 12536217.
3. Hattori T, Taft J M, Swist K M, Luo H, Witt H, Slattery M, Koide A, Ruthenburg A J, Krajewski K, Strahl B D, White K P, Farnham P J, Zhao Y, Koide S. Recombinant antibodies to histone post-translational modifications. Nat Methods. 2013; 10(10):992-5. doi: 10.1038/nmeth.2605. PubMed PMID: 23955773; PMCID: 3828030.
4. Nishikori S, Hattori T, Fuchs S M, Yasui N, Wojcik J, Koide A, Strahl B D, Koide S. Broad ranges of affinity and specificity of anti-histone antibodies revealed by a quantitative Peptide immunoprecipitation assay. J Mol Biol. 2012; 424(5):391-9. Epub 2012 Oct. 9. doi: 10.1016/j.jmb.2012.09.022. PubMed PMID: 23041298; PMCID: 3502729.

### Example 3

This Example describes antibodies that bind to peptide-drug conjugates, but only in the context of specific MHC display of the described peptide-drug conjugates. The antibodies were produced as follows.

### Antigen Preparation

KRAS(G12C) peptides ((H2N-VVGACGVGK-OH (SEQ ID NO: 1) and H2N-VVVGACGVGK-OH (SEQ ID NO: 2)) were reacted with AMG510 (Selleckchem) and loaded onto Flex-T HLA-A*03:01 and Flex-T HLA-A*11:01 (produced by Biolegend), or onto HLA-A*03:01 and HLA-A*11:01 produced in house. KRAS(WT) peptide ((H2N-VVGAGGVGK-OH (SEQ ID NO: 29)) was loaded onto the HLA molecules in the same manner. EGFR peptide (H2N-QLMPFGCLL-OH (SEQ ID NO: 30)) was reacted with Osimertinib (Selleckchem) and loaded onto Flex-T HLA-A*02:01 or HLA-A*02:01 produced in house. As a control, the same peptide was reacted with beta-mercaptoethanol and loaded onto the HLA molecule. BTK peptide (H2N-YMANGCLLNY-OH (SEQ ID NO: 31)) was reacted with Ibrutinib (Selleckchem) and loaded onto Flex-T HLA-A*01:01 or HLA-A*01:01 produced in house. As a control, the same peptide was reacted with beta-mercaptoethanol and loaded onto the HLA molecule. The peptide-loaded HLA mixtures prepared with Flex-T HLA proteins were used without further purification. The peptide-loaded HLA mixtures prepared with HLA samples prepared in house were further purified using size-exclusion chromatography with a Superdex S200 column.

### Antibody Phage-Display Library Sorting

Sorting of an antibody phage-display library was performed as described previously⁽¹⁾. Briefly, a phage-display library was first sorted with all four antigens at 100 nM in the first round, followed by sorting with a single antigen at 100, 50, and 20 nM in the second, third, and fourth rounds, respectively. To enrich for clones with the desired specificity, counterselection was performed using KRAS(WT) peptide-loaded MHC molecules or beta-mercaptoethanol-treated peptide-loaded MHC molecules in the second, third, and fourth rounds.

Binding of individual phage clones were tested using the multiplex bead binding assay⁽²⁾.

### Antibody Yeast-Display Library Sorting and Clone Characterization

Display of antibody clones in the form of single-chain Fv (scFv) on the yeast surface, library sorting using fluorescence-activated cell sorting, and characterization of individual clones were performed essentially as described previously (Hattori et al. PMID 23955773; Cao et al. PMID 17406305).

### Deep Mutational Scanning

Deep mutational scanning was performed following general procedures published previously (PMID 32841599). A yeast-display library, in the scFv format, contained variants in which a single position was diversified with the NNK codon. A yeast display libray was subjected to FACS using an antigen of interest to enrich a pool of clones that bound the antigen and a pool of clones that did not bind the antigen. The DNA sequences of the enriched pools were determined, and amino acid substitutions were deduced.

### Antibody Production

The genes encoding selected antibody clones were transferred from the phage-display vector to IgG expression vectors (pFUSEss-CHIg-hG1 and pFUSE2ss-CLIg-hK, InvivoGen), and IgG proteins were produced using the ExpiCHO cell line (Thermo Fisher) and purified using a Protein Capture Devices with Protein A (GORE).

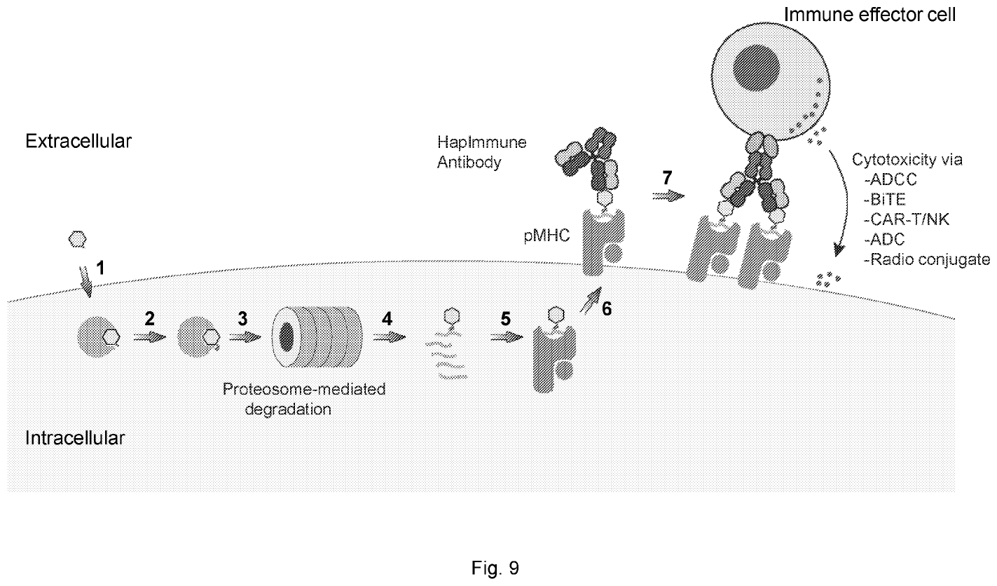

Data presented in this Example relates to FIGS. 9-15, 17, which provide the following information:

FIG. 9 provides a cartoon representation of a concept of the disclosure referred to as HapImmune. The numbers 1-7 denote relevant steps. 1. A covalent inhibitor is administered, and it enters the cell harboring the target protein. 2. The inhibitor binds the target and forms a covalent bond with the target. 3 and 4. As a part of natural protein turnover (or induced protein degradation in the case of a PROTAC), the target-drug conjugate is degraded by the proteosome system. As a result, peptides with the conjugated drug are produced. 5. A peptide conjugate is incorporated into a compatible MHC molecule. 6. The MHC/peptide-drug conjugate complex translocates to the surface of the cell. A HapImmune antibody recognizes the complex. 7. The surface bound antibody recruits an immune effector cell, such as an NK cell, which in turn initiates cell killing activities. Multiple modalities are envisioned for effecting cell killing activities, including ADCC, ADCP, CDC, BiTE, CAR-T, CAR-NK, ADC, and radioisotope conjugate, but they are not explicitly depicted here.

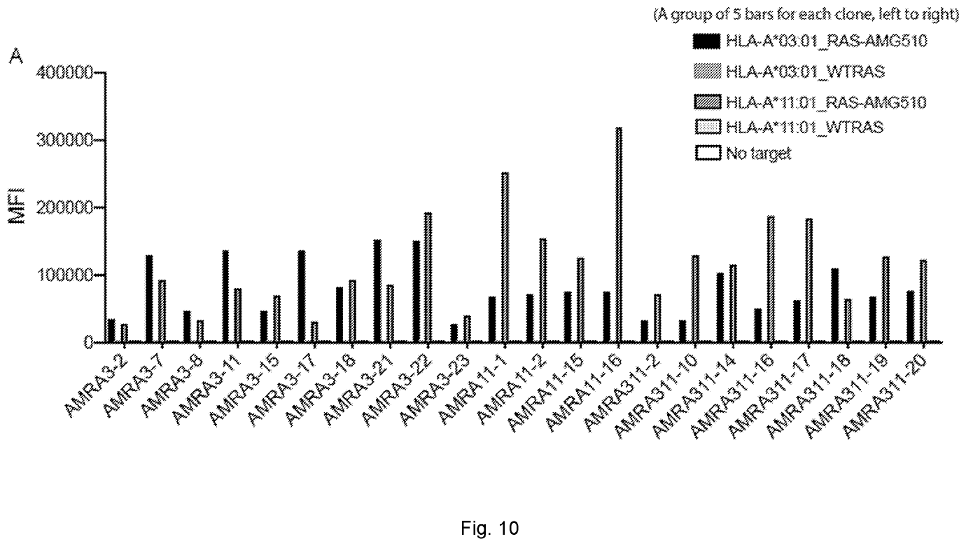

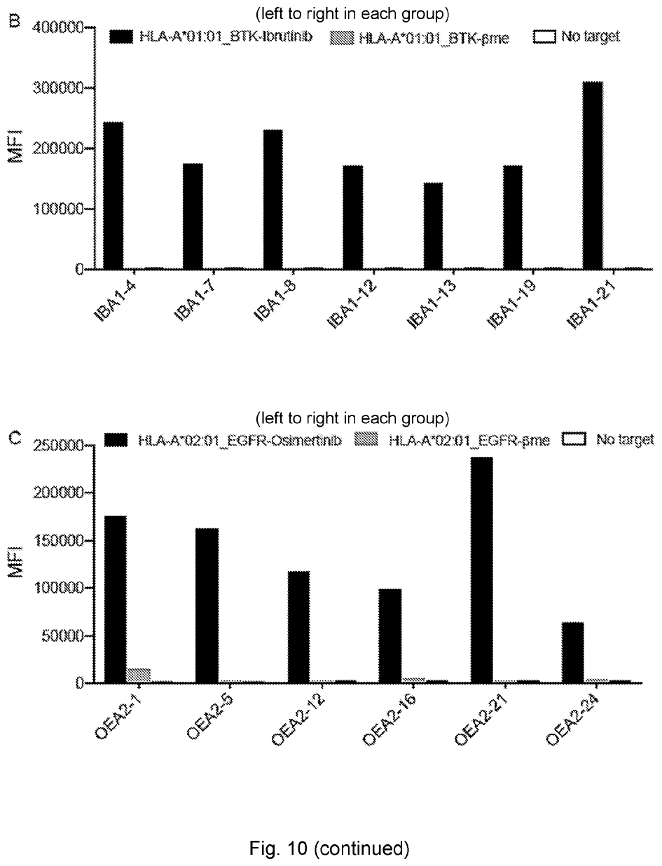

FIG. 10 shows data from development of antibodies that bind MHC/peptide-drug conjugate complexes. (A) Multiplex bead-binding assay (MBBA)⁽¹⁾ of phages displaying different antibody clones. For each phage clone, binding to a total of five antigens presented on beads was tested: HLA-A*03:01 in complex with the KRAS(G12C) peptide conjugated with AMG510 (denoted as HLA-A*03:01_RAS-AMG510 in the figure); HLA-A*03:01 in complex with the KRAS(wild type) peptide (denoted as HLA-A*03:01_WTRAS); HLA-A*11:01 in complex with the KRAS(G12C) peptide conjugated with AMG510 (denoted as HLA-A*11:01_RAS-AMG510); HLA-A*11:01 in complex with the KRAS(wild type) peptide (denoted as HLA-A*11:01_WTRAS); beads presenting no antigen (denoted as No target). (B) MBBA assay of phages displaying different antibody clones to: HLA-A*01:01 in complex with the BTK peptide conjugated with Ibrutinib (denoted as HLA-A*01:01_BTK-Ibrutinib in the figure); HLA-A*01:01 in complex with the BTK peptide conjugated with beta-mercaptoethanol (denoted as HLA-A*01:01_BTK-Dme); beads presenting no antigen (denoted as No target). (C) MBBA assay of phages displaying different antibody clones to: HLA-A*02:01 in complex with the EGFR peptide conjugated with Osimertinib (denoted as HLA-A*02:01_EGFR-Osimertinib in the figure); HLA-A*02:01 in complex with the EGFR peptide conjugated with beta-mercaptoethanol (denoted as HLA-A*02:01_EGFR-βme); beads presenting no antigen (denoted as No target).

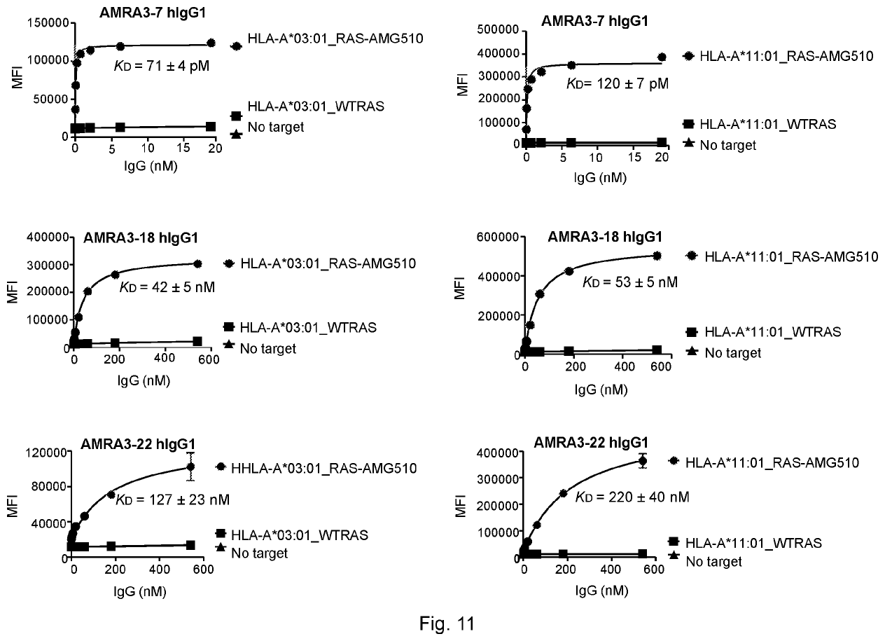

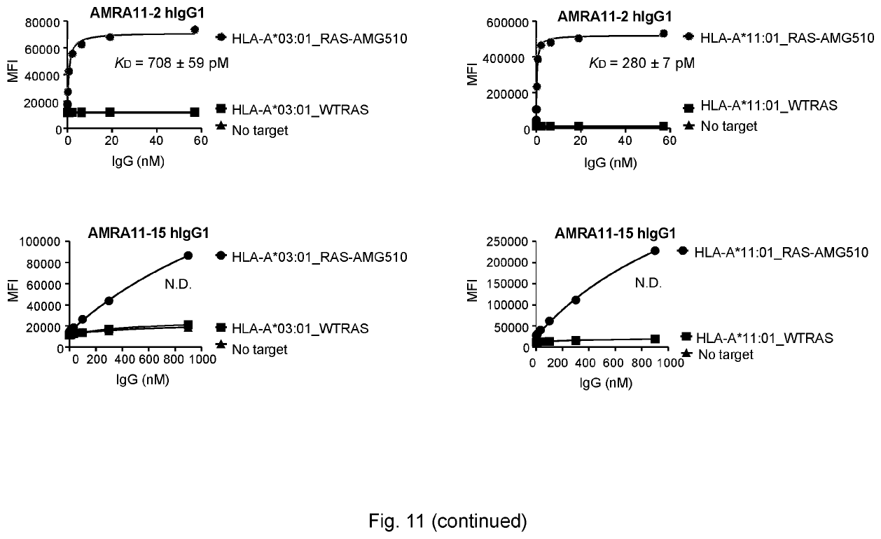

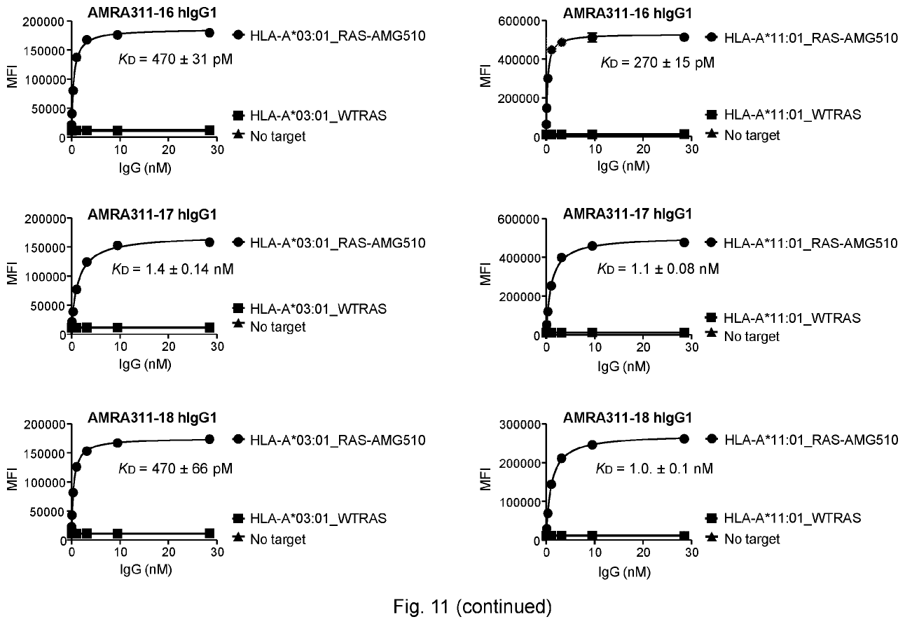

FIG. 11 shows results from binding titration using the multiplex bead-binding assay (MBBA) of purified antibodies targeted to the KRAS(G12C)-AMG510 conjugate. Clone names are shown over each graph. Antigen nomenclature is described in FIG. 10. The left column shows binding data with HLA-A*03:01 complexes, whereas the right column shows data with HLA-A*11:01 complexes. Apparent dissociation constant (K_{D}) values were determined using nonlinear least-squared fitting of a 1:1 binding function. The data for the wild-type RAS peptide complexes and for the no target were all close to the baseline and overlap, and thus their apparent K_{D} values were not determined. Data shown here are from triplicate measurements. Error bars are within the size of the symbols.

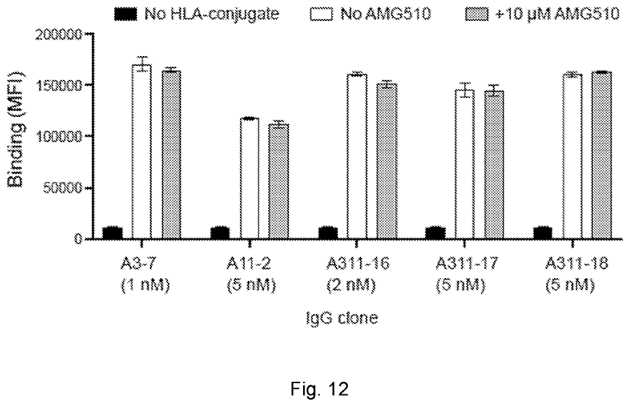

FIG. 12 demonstrates that binding of antibodies to the drug-peptide conjugate in complex with an MHC was not affected by the presence of the free drug. MBBA binding signals of select “AMR” series antibodies to HLA-A*03:01 in complex with the KRAS(G12C) peptide conjugated with AMG510 in the absence (the white bars) and presence (the gray bars) of 10 μM free AMG510 are shown. The antibody concentrations were adjusted to give sub-saturating signals and are shown in parentheses. Data shown here are from triplicate measurements.

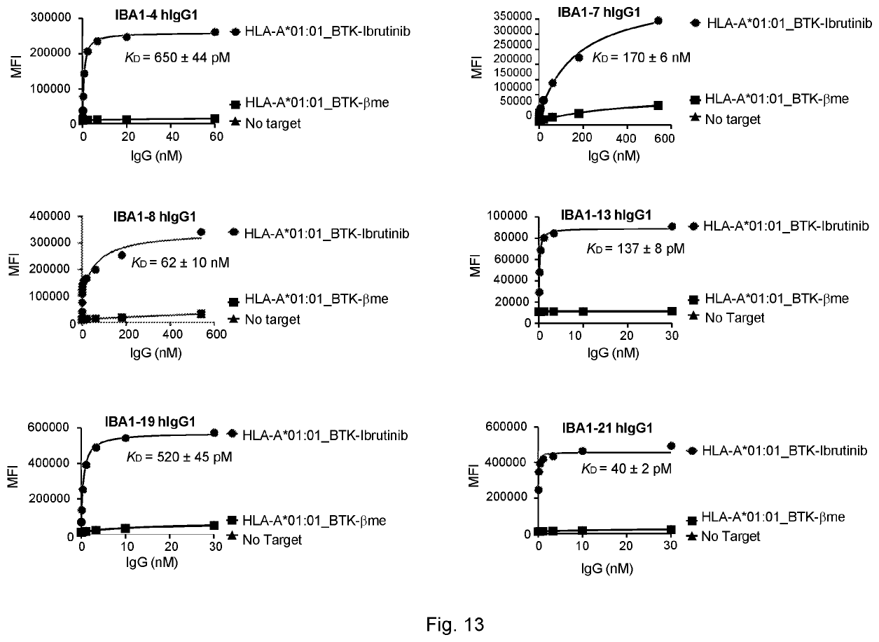

FIG. 13 shows results from binding titrations using the multiplex bead-binding assay (MBBA) of purified antibodies targeted to the BTK-Ibrutinib conjugate. Clone names are shown over each graph. Antigen nomenclature is described in FIG. 10. Apparent dissociation constant (K_{D}) values were determined using nonlinear least-squared fitting of a 1:1 binding function. The data for the beta-mercaptoethanol-conjugated peptide in complex with HLA-A*01:01 and for the no target were all close to the baseline and overlap, and thus their apparent K_{D} values were not determined. Data shown here are from triplicate measurements. Error bars are within the size of the symbols.

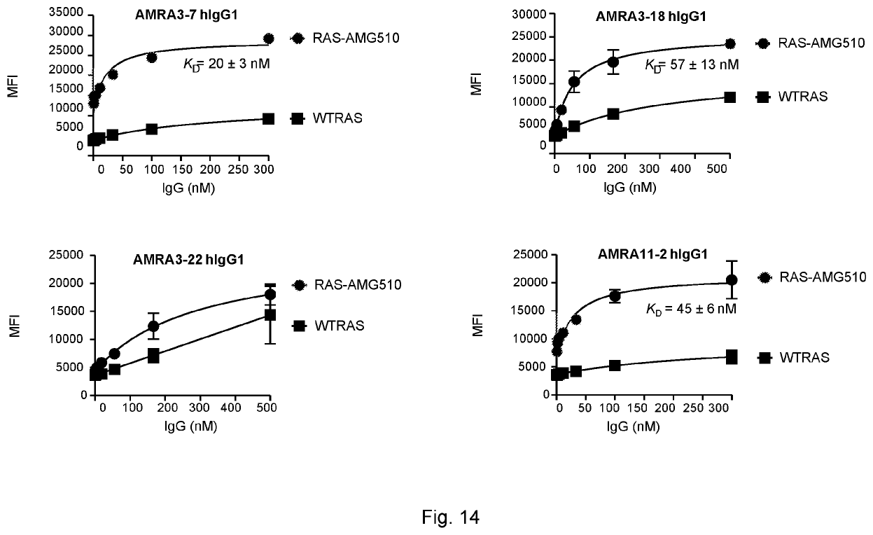

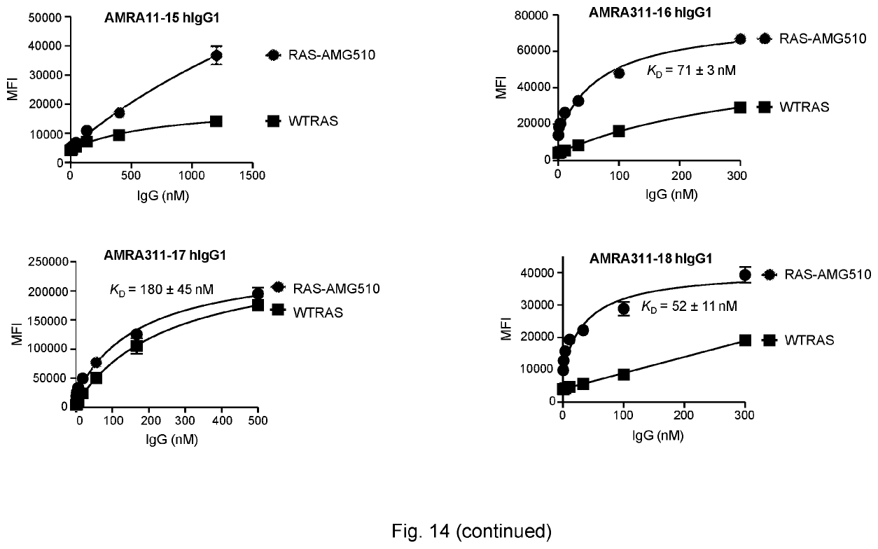

FIG. 14 shows results from binding titrations of purified antibodies to the KRAS(G12C)-AMG510 conjugate presented by endogenous MHC molecules on the cell surface. Raji cells were first incubated with the KRAS(G12C)-AMG510 conjugate or the KRAS(wild type) peptide, and excess conjugate and peptide were washed away. Surface-bound antibody levels detected using a fluorescently labeled secondary antibody are shown as a function of IgG concentration used for staining. Apparent dissociation constant (K_{D}) values were determined using nonlinear least-squared fitting of a 1:1 binding function. Data shown here are from triplicate measurements.

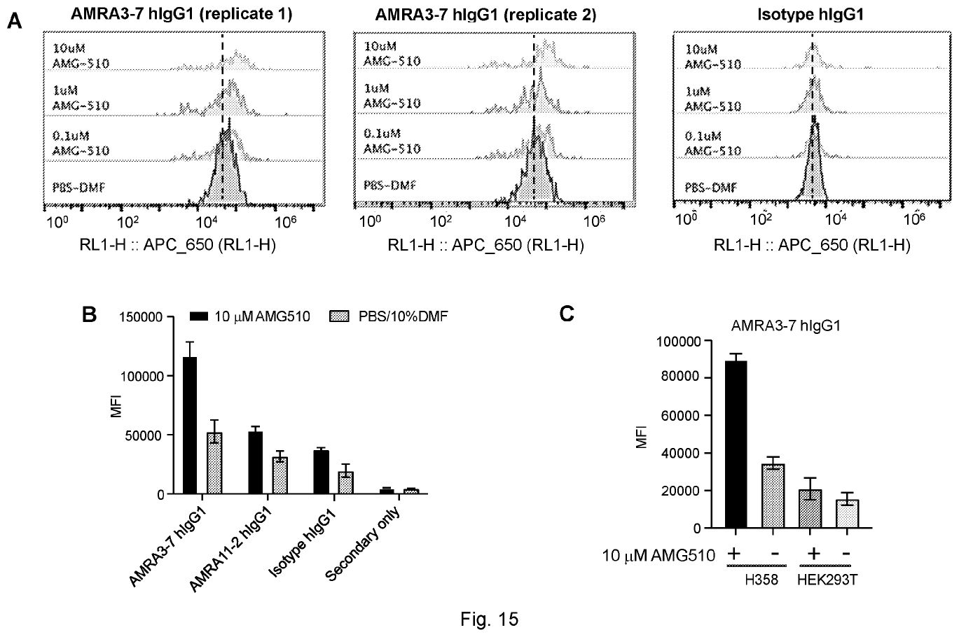

FIG. 15 shows results from an antibody binding to a KRAS(G12C)-expressing cell line pretreated with AMG510. The non-small cell lung cancer cell line H358 was incubated with AMG510 for 2 days and then stained with antibodies targeting the KRAS(G12C) peptide-AMG510 conjugate or an isotype control, followed by detection with a secondary antibody. (A) Flow cytometry histograms. (B) Quantification of the median fluorescence intensity of H358 cells treated with or without AMG510. The antibodies used are indicated along the horizontal axis. (C) Quantification of the median fluorescence intensity of H358 cells and HEK293T cells (a negative control) treated with or without AMG510 and stained with the AMRA3-7 antibody.

FIG. 16 is related to Example 2 and shows binding of P2AMR-1 IgG to cells preincubated with the KRAS(G12C) peptide-AMG510 conjugate, KRAS(wild type) peptide, or no peptide. The antibody was precomplexed with a dye-labeled secondary antibody in order to enhance the effective binding (avidity). The antibody bound to the Raji cells that express HLA-A*03:01 when the cells were incubated with the conjugate.

FIG. 17 shows results from binding of purified antibodies in the IgG format to the indicated drug-peptide/MHC complexes as measured using the multiplex bead binding assay (MBBA).

FIGS. 18 and 19 are discussed in Example 4.

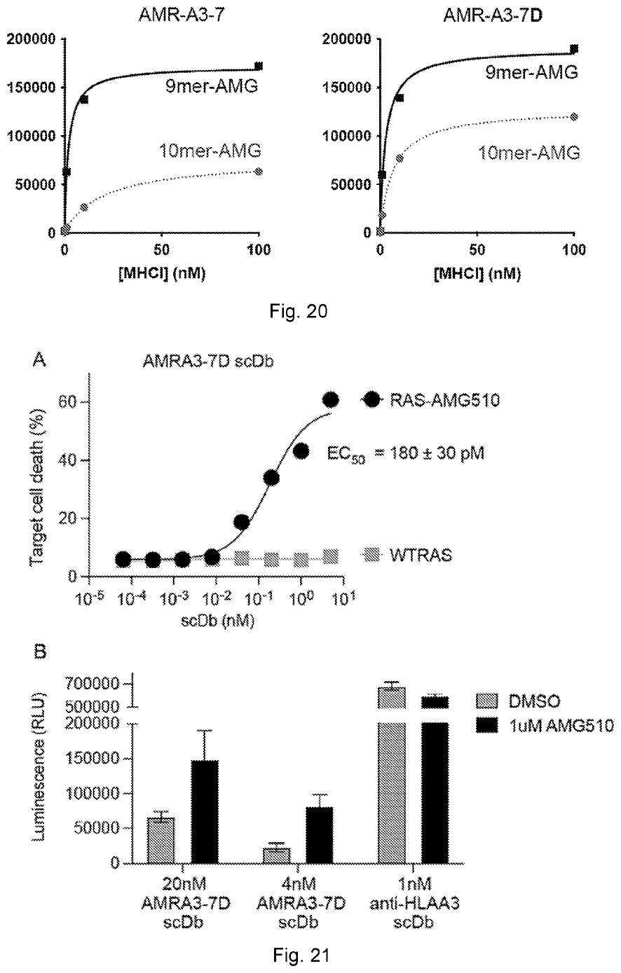

FIG. 20 shows results from binding titration curves of AMR-A3-7 and AMR-A3-7D displayed on the yeast surface. Binding to HLA-A*03:01 presenting AMG510 conjugated to the Cys residue in the 9mer and 10mer RAS(G12C) peptides, VVGACGVGK (SEQ ID NO: 1) and VVVGACGVGK (SEQ ID NO: 2), respectively, is shown.

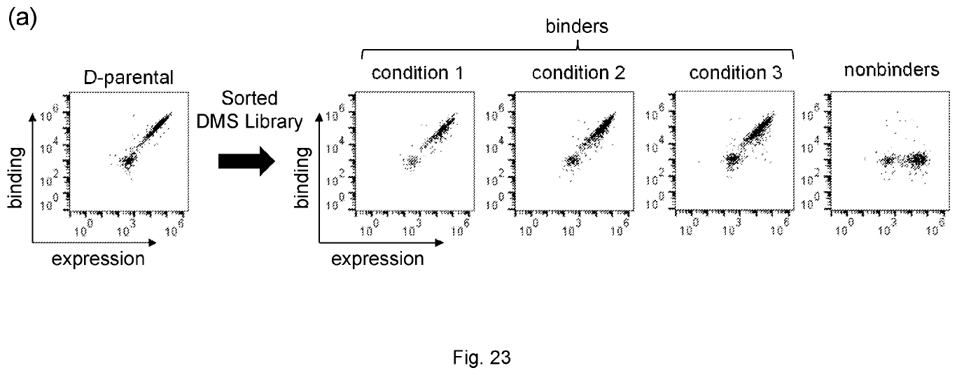

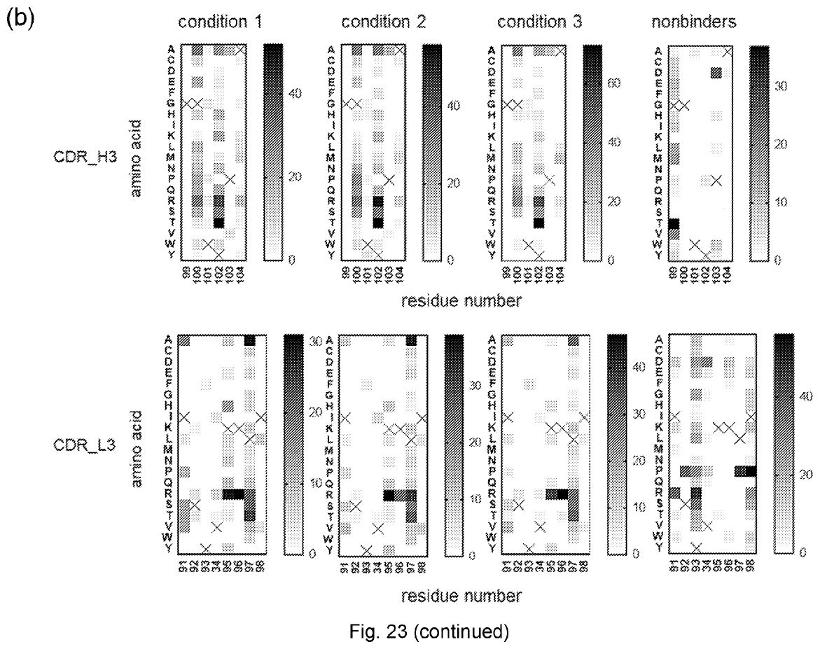

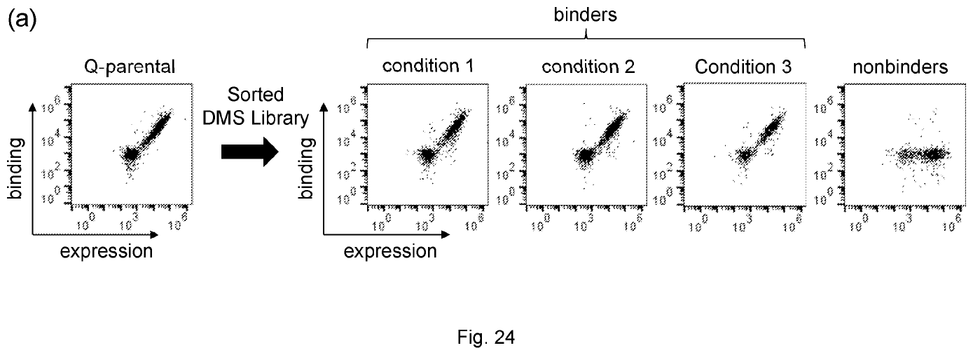

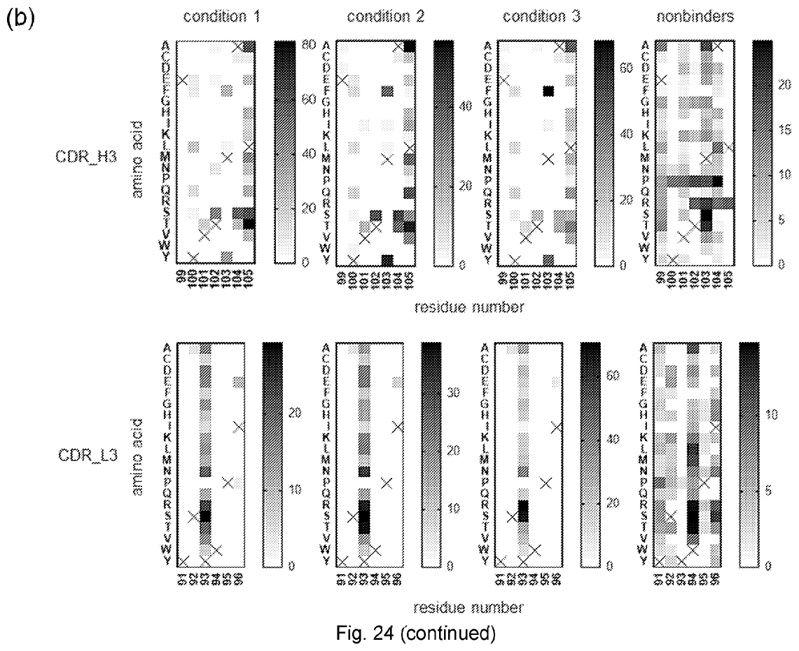

FIGS. 23 and 24 show results from deep mutational scanning of CDR-L3 and CDR-H3 of AMR-A3-7D and OEA2-5, respectively.

Specific and non-limiting examples of antibody sequences that bind in an MHC-drug conjugate-specific manner are as follows:

Exemplary Antibody Clones Binding to KRAS(G12C)-AMG510 conjugate presented on HLA-A*03:01 and HLA-A*11:01. CDR residues (Kabat scheme) in bold.

AMRA3-2
(SEQ ID NO: 32)
V_{L}:DIQMTQSPSSLSASVGDRVTITCRASQSVSSAVAWYQQKPGKAPKL
LIYSASSLYSGVPSRFSGSRSGTDFTLTISSLQPEDFATYYCQQSGWSY
PITFGQGTKVEIKRTV
V_{H}:
(SEQ ID NO: 33)
EVQLVESGGGLVQPGGSLRLSCAASGFTFYSSYIHWVRQAPGKGLEWVA
SISPYYGSTSYADSVKGRFTISADTSKNTAYLQMNSLRAEDTAVYYCAR
SSYYALDYWGQGTLVTVSS
AMRA3-7
V_{L}:
(SEQ ID NO: 34)
DIQMTQSPSSLSASVGDRVTITCRASQSVSSAVAWYQQKPGKAPKLLIY
SASSLYSGVPSRFSGSRSGTDFTLTISSLQPEDFATYYCQQISYVYSLI
TFGQGTKVEIKRTV
V_{H}:
(SEQ ID NO: 35)
EVQLVESGGGLVQPGGSLRLSCAASGFTFSSYSIHWVRQAPGKGLEWVA
SIYSSYGYTSYADSVKGRFTISADTSKNTAYLQMNSLRAEDTAVYYCAR
GGWYPAMDYWGQGTLVTVSS
AMRA3-7KK
V_{L}:
(SEQ ID NO: 36)
DIQMTQSPSSLSASVGDRVTITCRASQSVSSAVAWYQQKPGKAPKLLIY
SASSLYSGVPSRFSGSRSGTDFTLTISSLQPEDFATYYCQQISYVKKLI
TFGQGTKVEIKRTV
V_{H}:
(SEQ ID NO: 37)
EVQLVESGGGLVQPGGSLRLSCAASGFTFSSYSIHWVRQAPGKGLEWVA
SIYSSYGYTSYADSVKGRFTISADTSKNTAYLQMNSLRAEDTAVYYCAR
GGWYPAMDYWGQGTLVTVSS
AMRA3-7D
V_{L}:
(SEQ ID NO: 3)
DIQMTQSPSSLSASVGDRVTITCRASQSVSSAVAWYQQKPGKAPKLLIY
SASSLYSGVPSRFSGSRSGTDFTLTISSLQPEDFATYYCQQISYVKKLI
TFGQGTKVEIKRTV
V_{H}:
(SEQ ID NO: 4)
EVQLVESGGGLVQPGGSLRLSCAASGFTFSDYSIHWVRQAPGKGLEWVA
SISSSSGSTSYADSVKGRFTISADTSKNTAYLQMNSLRAEDTAVYYCAR
GGWYPAMDYWGQGTLVTVSS
AMRA3-8
V_{L}:
(SEQ ID NO: 38)
DIQMTQSPSSLSASVGDRVTITCRASQSVSSAVAWYQQKPGKAPKLLIY
SASSLYSGVPSRFSGSRSGTDFTLTISSLQPEDLATYYCQQYQYGYNLI
TFGQGTKVEIKRTV
V_{H}:
(SEQ ID NO: 39)
EVQLVESGGGLVQPGGSLRLSCAASGFTISYSSIHWVRQAPGKGLEWVA
SIYSYSGSTSYADSVKGRFTISADTSKNTAYLQMNSLRAEDTAVYYCAR
YSYGWVGPGWRAIDYWGQGTLVTVSS
AMRA3-11
V_{L}:
(SEQ ID NO: 40)
DIQMTQSPSSLSASVGDRVTITCRASQSVSSAVAWYQQKPGKAPKLLIY
SASSLYSGVPSRFSGSRSGTDFTLTISSLQPEDFATYYCQQSSSVYKLL
TFGQGTKVEIKRTV
V_{H}:
(SEQ ID NO: 41)
EVQLVESGGGLVQPGGSLRLSCAASGFTVYYSSIHWVRQAPGKGLEWVA
SISSSYSYTSYADSVKGRFTISADTSKNTAYLQMNSLRAEDTALYYCAR
GGPGWYRAMDYWGQGTLVTVSS
AMRA3-15
V_{L}:
(SEQ ID NO: 42)
DIQMTQSPSSLSASVGDRVTITCRASQSVSSAVAWYQQKPGKAPKLLIY
SASSLYSGVPSRFSGSRSGTDFTLTISSLQPEDFATYYCQQSSSSLITF
GQGTKVEIKRTV
V_{H}:
(SEQ ID NO: 43)
EVQLVESGGGLVQPGGSLRLSCAASGFTFSSSSIHWVRQAPGKGLEWVA
SISSSSGSTSYADSVKGRFTISADTSKNTAYLQMNSLRAEDTAVYYCAR
GYFYYGWWAMAFDYWGQGTLVTVSS
AMRA3-17
V_{L}:
(SEQ ID NO: 44)
DIQMTQSPSSLSASVGDRVTITCRASQSVSSAVAWYQQKPGKAPKLLIY
SASSLYSGVPSRFSGSRSGTDFTLTISSLQPEDFATYYCQQSQWYEPLI
TFGQGTKVEIKRTV
V_{H}:
(SEQ ID NO: 45)
EVQLVESGGGLVQPGGSLRLSCAASGFTIYSSYIHWVRQAPGKGLEWVA
SISSSSGSTSYADSVKGRFTISADTSKNTAYLQMNSLRAEDTAVYYCAR
SYSYMSQWGWYQYSGMDYWGQGTLVTVSS
AMRA3-18
V_{L}:
(SEQ ID NO: 46)
DIQMTQSPSSLSASVGDRVTITCRASQSVSSAVAWYQQKPGKAPKLLIY
SASSLYSGVPSRFSGSRSGTDFTLTISSLQPEDFATYYCQQGSYTYRLI
TFGQGTKVEIKRTV
V_{H}:
(SEQ ID NO: 47)
EVQLVESGGGLVQPGGSLRLSCAASGFTVSYSSIHWVRQAPGKGLEWVA
SISSSSGYTSYADSVKGRFTISADTSKNTAYLQMNSLRAEDTAVYYCAR
YAWWAHGLDYWGQGTLVTVSS
AMRA3-21
V_{L}:
(SEQ ID NO: 48)
DIQMTQSPSSLSASVGDRVTITCRASQSVSSAVAWYQQKPGKAPKLLIY
SASSLYSGVPSRFSGSRSGTDFTLTISSLQPEDFATYYCQQASYWYNLF
TFGQGTKVEIKRTV
V_{H}:
(SEQ ID NO: 49)
EVQLVESGGGLVQPGGSLRLSCAASGFTISSYSIHWVRQAPGKGLEWVA
SIYSSYGYTSYADSVKGRFTISADTSKNTAYLQMNSLRAEDTAVYYCAR
QYSMHFPWGYGMDYWGQGTLVTVSS
AMRA3-22
V_{L}:
(SEQ ID NO: 50)
DIQMTQSPSSLSASVGDRVTITCRASQSVSSAVAWYQQKPGKAPKLLIY
SASSLYSGVPSRFSGSRSGTDFTLTISSLQPEDFATYYCQQSDMPPITF
GQGTKVEIKRTV
V_{H}:
(SEQ ID NO: 51)
EVQLVESGGGLVQPGGSLRLSCAASGFTFYSSSIHWVRQAPGKGLEWVA
YIYSSSGYTSYADSVKGRFTISADTSKNTAYLQMNSLRAEDTAVYYCAR
PVNYYYQGALDYWGQGTLVTVSS
AMRA3-23
V_{L}:
(SEQ ID NO: 52)
DIQMTQSPSSLSASVGDRVTITCRASQSVSSAVAWYQQKPGKAPKLLIY
SASSLYSGVPSRFSGSRSGTDFTLTISSLQPEDFATYYCQQYYVFPITF
GQGTKVEIKRTV
V_{H}:
(SEQ ID NO: 53)
EVQLVESGGGLVQPGGSLRLSCAASGFTVYSSSIHWVRQAPGKGLEWVA
SISPSSGYTYYADSVKGRFTISADTSKNTAYLQMNSLRAEDTAVYYCAR
YHYMFEYDKGESKWGYYGFDYWGQGTLVTVSS
AMRA11-1
V_{L}:
(SEQ ID NO: 54)
DIQMTQSPSSLSASVGDRVTITCRASQSVSSAVAWYQQKPGKAPKLLIY
SASSLYSGVPSRFSGSRSGTDFTLTISSLQPEDFATYYCQQSQYFPITF
GQGTKVEIKRTV
V_{H}:
(SEQ ID NO: 55)
EVQLVESGGGLVQPGGSLRLSCAASGFTISYSSIHWVRQAPGKGLEWVA
SIYSYYGYTSYADSVKGRFTISADTSKNTAYLQMNSLRAEDTAVYYCAR
NSWSWYSGVGMDYWGQGTLVTVSS
AMRA11-2
V_{L}:
(SEQ ID NO: 56)
DIQMTQSPSSLSASVGDRVTITCRASQSVSSAVAWYQQKPGKAPKLLIY
SASSLYSGVPSRFSGSRSGTDFTLTISSLQPEDFATYYCQQSSSSLITF
GQGTKVEIKRTV
V_{H}:
(SEQ ID NO: 57)
EVQLVESGGGLVQPGGSLRLSCAASGFTISSSSIHWVRQAPGKGLEWVA
SISSYSSSTYYADSVKGRFTISADTSKNTAYLQMNSLRAEDTAVYYCAR
YPYGWGWGGSGLDYWGQGTLVTVSS
AMRA11-15
VL:
(SEQ ID NO: 58)
DIQMTQSPSSLSASVGDRVTITCRASQSVSSAVAWYQQKPGKAPKLLIY
SASSLYSGVPSRFSGSRSGTDFTLTISSLQPEDFATYYCQQFDFQYLIT
FGQGTKVEIKRTV
V_{H}:
(SEQ ID NO: 59)
EVQLVESGGGLVQPGGSLRLSCAASGFTVYYSSIHWVRQAPGKGLEWVA
SIYSYYGSTYYADSVKGRFTISADTSKNTAYLQMNSLRAEDTAVYYCAR
GEKWALDYWGQGTLVTVSS
AMRA11-16
V_{L}:
(SEQ ID NO: 60)
DIQMTQSPSSLSASVGDRVTITCRASQSVSSAVAWYQQKPGKAPKLLIY
SASSLYSGVPSRFSGSRSGTDFTLTISSLQPEDFATYYCQQYMYYQPLI
TFGQGTKVEIKRTV
V_{H}:
(SEQ ID NO: 61)
EVQLVESGGGLVQPGGSLRLSCAASGFTVYYSSIHWVRQAPGKGLEWVA
SISSSSGSTSYADSVKGRFTISADTSKNTAYLQMNSLRAEDTAVYYCAR
EPYNYNWYGMDYWGQGTLVTVSS
AMRA311-2
V_{L}:
(SEQ ID NO: 62)
DIQMTQSPSSLSASVGDRVTITCRASQSVSSAVAWYQQKPGKAPKLLIY
SASSLYSGVPSRFSGSRSGTDFTLTISSLQPEDFATYYCQQSLWWPITF
GQGTKVEIKRTV
V_{H}:
(SEQ ID NO: 63)
EVQLVESGGGLVQPGGSLRLSCAASGFTVSSSSIHWVRQAPGKGLEWVA
SIYSYSGYTSYADSVKGRFTISADTSKNTAYLQMNSLRAEDTAVYYCAR
HGSYGSWWALDYWGQGTLVTVSS
AMRA311-10
V_{L}:
(SEQ ID NO: 64)
DIQMTQSPSSLSASVGDRVTITCRASQSVSSAVAWYQQKPGKAPKLLIY
SASSLYSGVPSRFSGSRSGTDFTLTISSLQPEDFATYYCQQYFYFPITF
GQGTKVEIKRTV
V_{H}:
(SEQ ID NO: 65)
EVQLVESGGGLVQPGGSLRLSCAASGFTFYSSSIHWVRQAPGKGLEWVA
SISSYYGSTSYADSVKGRFTISADTSKNTAYLQMNSLRAEDTAVYYCAR
ASYYSGYGSSYPYYMGLDYWGQGTLVTVSS
AMRA311-14
V_{L}:
(SEQ ID NO: 66)
DIQMTQSPSSLSASVGDRVTITCRASQSVSSAVAWYQQKPGKAPKLLIY
SASSLYSGVPSRFSGSRSGTDFTLTISSLQPEDFATYYCQQGSYRNPLL
TFGQGTKVEIKRTV
V_{H}:
(SEQ ID NO: 67)
EVQLVESGGGLVQPGGSLRLSCAASGFTFSSYSIHWVRQAPGKGLEWVA
SISSSSGYTSYADSVKGRFTISADTSKNTAYLQMNSLRAEDTAVYYCAR
MNWSHYAMDYWGQGTLVTVSS
AMRA311-16
V_{L}:
(SEQ ID NO: 68)
DIQMTQSPSSLSASVGDRVTITCRASQSVSSAVAWYQQKPGKAPKLLIY
SASSLYSGVPSRFSGSRSGTDFTLTISSLQPEDFATYYCQQSSSSLITF
GQGTKVEIKRTV
V_{H}:
(SEQ ID NO: 69)
EVQLVESGGGLVQPGGSLRLSCAASGFTISSSSIHWVRQAPGKGLEWVA
YISSYSGYTYYADSVKGRFTISADTSKNTAYLQMNSLRAEDTAVYYCAR
YWYGHYHSYFGLDYWGQGTLVTVSS
AMRA311-17
V_{L}:
(SEQ ID NO: 70)
DIQMTQSPSSLSASVGDRVTITCRASQSVSSAVAWYQQKPGKAPKLLIY
SASSLYSGVPSRFSGSRSGTDFTLTISSLQPEDFATYYCQQSSSSLITF
GQGTKVEIKRTV
V_{H}:
(SEQ ID NO: 71)
EVQLVESGGGLVQPGGSLRLSCAASGFTISSSSIHWVRQAPGKGLEWVA
SISSYSGSTYYADSVKGRFTISADTSKNTAYLQMNSLRAEDTAVYYCAR
YPYGSHVYTGLDYWGQGTLVTVSS
AMRA311-18
V_{L}:
(SEQ ID NO: 72)
DIQMTQSPSSLSASVGDRVTITCRASQSVSSAVAWYQQKPGKAPKLLIY
SASSLYSGVPSRFSGSRSGTDFTLTISSLQPEDFATYYCQQWNWADYLV
TFGQGTKVEIKRTV
V_{H}:
(SEQ ID NO: 73)
EVQLVESGGGLVQPGGSLRLSCAASGFTISSSSIHWVRQAPGKGLEWVA
SIYSSSGSTSYADSVKGRFTISADTSKNTAYLQMNSLRAEDTAVYYCAR
VYSSRYWGWGVAFDYWGQGTLVTVSS
AMRA311-19
V_{L}:
(SEQ ID NO: 74)
DIQMTQSPSSLSASVGDRVTITCRASQSVSSAVAWYQQKPGKAPKLLIY
SASSLYSGVPSRFSGSRSGTDFTLTISSLQPEDFATYYCQQYYWYSLIT
FGQGTKVEIKRTV
V_{H}:
(SEQ ID NO: 75)
EVQLVESGGGLVQPGGSLRLSCAASGFTVYSSSIHWVRQAPGKGLEWVA
YIYSSSGYTSYADSVKGRFTISADTSKNTAYLQMNSLRAEDTAVYYCAR
RSFPQWYNGSYTPWPAMDYWGQGTLVTVSS
AMRA311-20
V_{L}:
(SEQ ID NO: 76)
DIQMTQSPSSLSASVGDRVTITCRASQSVSSAVAWYQQKPGKAPKLLIY
SASSLYSGVPSRFSGSRSGTDFTLTISSLQPEDFATYYCQQYMWWPVTF
GQGTKVEIKRTV
V_{H}:
(SEQ ID NO: 77)
EVQLVESGGGLVQPGGSLRLSCAASGFTVSSSSIHWVRQAPGKGLEWVA
SIYSYSSYTSYADSVKGRFTISADTSKNTAYLQMNSLRAEDTAVYYCAR
PFYWGERYALDYWGQGTLVTVSS

Antibody clones that bind preferentially to KRAS(G12C)-AMG510 conjugate presented on HLA-A*03:01 relative to the same conjugate presented on HLA-A*11:01.

CDR residues (Kabat scheme) in bold.

AMRA3-5
V_{L}:
(SEQ ID NO: 78)
DIQMTQSPSSLSASVGDRVTITCRASQSVSSAVAWYQQKPGKAPKLLIY
SASSLYSGVPSRFSGSRSGTDFTLTISSLQPEDFATYYCQQSYSTLVTF
GQGTKVEIKRTV
V_{H}:
(SEQ ID NO: 79)
EVQLVESGGGLVQPGGSLRLSCAASGFTFYSSSIHWVRQAPGKGLEWVA
SIYSSYGSTSYADSVKGRFTISADTSKNTAYLQMNSLRAEDTAVYYCAR
IYGWSYQGWAGMDYWGQGTLVTVSS
AMRA3-6
V_{L}:
(SEQ ID NO: 80)
DIQMTQSPSSLSASVGDRVTITCRASQSVSSAVAWYQQKPGKAPKLLIY
SASSLYSGVPSRFSGSRSGTDFTLTISSLQPEDFATYYCQQSSSSLITF
GQGTKVEIKRTV
V_{H}:
(SEQ ID NO: 81)
EVQLVESGGGLVQPGGSLRLSCAASGFTISYSSIHWVRQAPGKGLEWVA
SIYPYYGSTSYADSVKGRFTISADTSKNTAYLQMNSLRAEDTAVYYCAR
GGDYYWGWYWVAMDYWGQGTLVTVSS
AMRA3-9
V_{L}:
(SEQ ID NO: 82)
DIQMTQSPSSLSASVGDRVTITCRASQSVSSAVAWYQQKPGKAPKLLIY
SASSLYSGVPSRFSGSRSGTDFTLTIXSLQPEDFATYYCQKSSSSLITF
GQGTKVEIKRTV
V_{H}:
(SEQ ID NO: 83)
EVQLVESGGGLVQPGGSLRLSCAASGFTFSSSYIHWVRQAPGKGLEWVA
SISSSYGSTSYADSVKGRFTISADTSKNTAYLQMNSLRAEDTAVYYCAR
MYYYTYPGMDYWGQGTLVTVSS
AMRA3-10
V_{L}:
(SEQ ID NO: 84)
DIQMTQSPSSLSASVGDRVTITCRASQSVSSAVAWYQQKPGKAPKLLIY
SASSLYSGVPSRFSGSRSGTDFTLTISSLQPEDFATYYCQKGSSYLLTF
GQGTKVEIKRTV
V_{H}:
(SEQ ID NO: 85)
EVQLVESGGGLVQPGGSLRLSCAASGFTIYSYSIHWVRQAPGKGLEWVA
SISPSSGYTSYADSVKGRFTISADTSKNTAYLQMNSLRAEDTAVYYCAR
YHYGGWSHYMSGMDYWGQGTLVTVSS
AMRA3-13
V_{L}:
(SEQ ID NO: 86)
DIQMTQSPSSLSASVGDRVTITCRASQSVSSAVAWYQQKPGKAPKLLIY
SASSLYSGVPSRFSGSRSGTDFTLTISSLQPEDFATYYCQQNYYYHKLI
TFGQGTKVEIKRTV
V_{H}:
(SEQ ID NO: 87)
EVQLVESGGGLVQPGGSLRLSCAASGFTFSYSSIHWVRQAPGKGLEWVA
SISSSYGYTSYADSVKGRFTISADTSKNTAYLQMNSLRAEDTAVYYCAR
GRYGGMDYWGQGTLVTVSS
AMRA3-19
V_{L}:
(SEQ ID NO: 88)
DIQMTQSPSSLSASVGDRVTITCRASQSVSSAVAWYQQKPGKAPKLLIY
SASSLYSGVPSRFSGSRSGTDFTLTISSLQPEDFATYYCQQLSYVYKLI
TFGQGTKVEIKRTV
V_{H}:
(SEQ ID NO: 89)
EVQLVESGGGLVQPGGSLRLSCAASGFTFYSSSIHWVRQAPGKGLEWVA
SISSSYGYTSYADSVKGRFTISADTSKNTAYLQMNSLRAEDTAVYYCAR
GWYKAMDYWGQGTLVTVSS
AMRA3-24
V_{L}:
(SEQ ID NO: 90)
DIQMTQSPSSLSASVGDRVTITCRASQSVSSAVAWYQQKPGKAPKLLIY
SASSLYSGVPSRFSGSRSGTDFTLTISSLQPEDFATYYCQQSSSSLITF
GQGTKVEIKRTV
V_{H}:
(SEQ ID NO: 91)
EVQLVESGGGLVQPGGSLRLSCAASGFTFSYSSIHWVRQAPGKGLEWVA
SISSSYGSTYYADSVKGRFTISADTSKNTAYLQMNSLRAEDTAVYYCAR
MYYYYYPGIDYWGQGTLVTVSS

Antibody clones that bind preferentially to KRAS(G12C)-AMG510 conjugate presented on HLA-A*11:01 relative to KRAS(G12C)-AMG510 conjugate presented on HLA-A*03:01.

AMRA11-3
V_{L}:
(SEQ ID NO: 92)
DIQMTQSPSSLSASVGDRVTITCRASQSVSSAVAWYQQKPGKAPKLLIY
SASSLYSGVPSRFSGSRSGTDFTLTISSLQPEDLATYYCQQYYYFPITF
GQGTKVEIKRTV
V_{H}:
(SEQ ID NO: 93)
EVQLVESGGGLVQPGGSLRLSCAASGFTFSSSSIHWVRQAPGKGLEWVA
SISPYYGYTSYADSVKGRFTISADTSKNTAYLQMNSLRAEDTAVYYCAR
SPYYWYQYFYGWGLDYWGQGTLVTVSS
AMRA11-4
V_{L}:
(SEQ ID NO: 94)
DIQMTQSPSSLSASVGDRVTITCRASQSVSSAVAWYQQKPGKAPKLLIY
SASSLYSGVPSRFSGSRSGTDFTLTISSLQPEDFATYYCQQSSSSLITF
GQGTKVEIKRTV
V_{H}:
(SEQ ID NO: 95)
EVQLVESGGGLVQPGGSLRLSCAASGFTFSYSSIHWVRQAPGKGLEWVA
SISSSSGSTSYADSVKGRFTISADTSKNTAYLQMNSLRAEDTAVYYCAR
SPYWWNYMSAMDYWGQGTLVTVSS
AMRA11-7
V_{L}:
(SEQ ID NO: 96)
DIQMTQSPSSLSASVGDRVTITCRASQSVSSAVAWYQQKPGKAPKLLIY
SASSLYSGVPSRFSGSRSGTDFTLTISSLQPEDFATYYCQQGWWWPFTF
GQGTKVEIKRTV
V_{H}:
(SEQ ID NO: 97)
EVQLVESGGGLVQPGGSLRLSCAASGFTVSSYSIHWVRQAPGKGLEWVA
SISPYYGYTSYADSVKGRFTISADTSKNTAYLQMNSLRAEDTAVYYCAR
WSWQYYSGHSSWGLDYWGQGTLVTVSS
AMRA11-8
V_{L}:
(SEQ ID NO: 98)
DIQMTQSPSSLSASVGDRVTITCRASQSVSSAVAWYQQKPGKAPKLLIY
SASSLYSGVPSRFSGSRSGTDFTLTISSLQPEDFATYYCQQSWYFPLTF
GQGTKVEIKRTV
V_{H}:
(SEQ ID NO: 99)
EVQLVESGGGLVQPGGSLRLSCAASGFTVSSSSIHWVRQAPGKGLEWVA
SIYSYYGYTSYADSVKGRFTISADTSKNTAYLQMNSLRAEDTAVYYCAR
WYNEYYHDYYWDAMDYWGQGTLVTVSS
AMRA11-9
V_{L}:
(SEQ ID NO: 100)
DIQMTQSPSSLSASVGDRVTITCRASQSVSSAVAWYQQKPGKAPKLLIY
SASSLYSGVPSRFSGSRSGTDFTLTISSLQPEDFATYYCQQSSSSLITF
GQGTKVEIKRTV
V_{H}:
(SEQ ID NO: 101)
EVQLVESGGGLVQPGGSLRLSCAASGFTLYYSSIHWVRQAPGKGLEWVA
SISSSSGSTSYADSVKGRFTISADTSKNTAYLQMNSLRAEDTAVYYCAR
WMYWWSFALDYWGQGTLVTVSS
AMRA11-10
V_{L}:
(SEQ ID NO: 102)
DIQMTQSPSSLSASVGDRVTITCRASQSVSSAVAWYQQKPGKAPKLLIY
SASSLYSGVPSRFSGSRSGTDFTLTISSLQPEDFATYYCQQSYLWPITF
GQGTKVEIKRTV
V_{H}:
(SEQ ID NO: 103)
EVQLVESGGGLVQPGGSLRLSCAASGFTVSSSSIHWVRQAPGKGLEWVA
SIYSYYGYTSYADSVKGRFTISADTSKNTAYLQMNSLRAEDTAVYYCAR
WQYHYNYWYGMDYWGQGTLVTVSS
AMRA11-11
V_{L}:
(SEQ ID NO: 104)
DIQMTQSPSSLSASVGDRVTITCRASQSVSSAVAWYQQKPGKAPKLLIY
SASSLYSGVPSRFSGSRSGTDFTLTISSLQPEDFATYYCQQYPMSLITF
GQGTKVEIKRTV
V_{H}:
(SEQ ID NO: 105)
EVQLVESGGGLVQPGGSLRLSCAASGFTVSYSSIHWVRQAPGKGLEWVA
SISPYSGYTSYADSVKGRFTISADTSKNTAYLQMNSLRAEDTAVYYCAR
GYDYYAGLDYWGQGTLVTVSS
AMRA11-12
V_{L}:
(SEQ ID NO: 106)
DIQMTQSPSSLSASVGDRVTITCRASQSVSSAVAWYQQKPGKAPKLLIY
SASSLYSGVPSRFSGSRSGTDFTLTISSLQPEDFATYYCQQYYYFPITF
GQGTKVEIKRTV
V_{H}:
(SEQ ID NO: 107)
EVQLVESGGGLVQPGGSLRLSCAASGFTFSYYSIHWVRQAPGKGLEWVA
SISPYYGYTSYADSVKGRFTISADTSKNTAYLQMNSLRAEDTAVYYCAR
WESEYSGTYEDYWAGMDYWGQGTLVTVSS
AMRA11-13
V_{L}:
(SEQ ID NO: 108)
DIQMTQSPSSLSASVGDRVTITCRASQSVSSAVAWYQQKPGKAPKLLIY
SASSLYSGVPSRFSGSRSGTDFTLTISSLQPEDFATYYCQQYMWWPITF
GQGTKVEIKRTV
V_{H}:
(SEQ ID NO: 109)
EVQLVESGGGLVQPGGSLRLSCAASGFTISYSSIHWVRQAPGKGLEWVA
SISSSYSYTSYADSVKGRFTISADTSKNTAYLQMNSLRAEDTAVYYCAR
TGYWQGYLALDYWGQGTLVTVSS
AMRA11-14
V_{L}:
(SEQ ID NO: 110)
DIQMTQSPSSLSASVGDRVTITCRASQSVSSAVAWYQQKPGKAPKLLIY
SASSLYSGVPSRFSGSRSGTDFTLTISSLQPEDFATYYCQQSSSSLITF
GQGTKVEIKRTV
V_{H}:
(SEQ ID NO: 111)
EVQLVESGGGLVQPGGSLRLSCAASGFTISYSSIHWVRQAPGKGLEWVA
SISSSSGSTSYADSVKGRFTISADTSKNTAYLQMNSLRAEDTAVYYCAR
TYYYYWNSTPAMDYWGQGTLVTVSS
AMRA11-18
V_{L}:
(SEQ ID NO: 112)
DIQMTQSPSSLSASVGDRVTITCRASQSVSSAVAWYQQKPGKAPKLLIY
SASSLYSGVPSRFSGSRSGTDFTLTISSLQPEDFATYYCQQSYGYPVTF
GQGTKVEIKRTV
V_{H}:
(SEQ ID NO: 113)
EVQLVESGGGLVQPGGSLRLSCAASGFTFSSSSIHWVRQAPGKGLEWVA
SISSSYGYTSYADSVKGRFTISADTSKNTAYLQMNSLRAEDTAVYYCAR
WYNSSWYYSNWWYKGFGMDYWGQGTLVTVSS
AMRA11-20
V_{L}:
(SEQ ID NO: 114)
DIQMTQSPSSLSASVGDRVTITCRASQSVSSAVAWYQQKPGKAPKLLIY
SASSLYSGVPSRFSGSRSGTDFTLTISSLQPEDFATYYCQQYYSSLFTF
GQGTKVEIKRTV
V_{H}:
(SEQ ID NO: 115)
EVQLVESGGGLVQPGGSLRLSCAASGFTFYSSSIHWVRQAPGKGLEWVA
SISSSYGYTSYADSVKGRFTISADTSKNTAYLQMNSLRAEDTAVYYCAR
TSYTYPVYTYYGFDYWGQGTLVTVSS
AMRA11-22
V_{L}:
(SEQ ID NO: 116)
DIQMTQSPSSLSASVGDRVTITCRASQSVSSAVAWYQQKPGKAPKLLIY
SASSLYSGVPSRFSGSRSGTDFTLTISSLQPEDFATYYCQQSWYYPLTF
GQGTKVEIKRTV
V_{H}:
(SEQ ID NO: 117)
EVQLVESGGGLVQPGGSLRLSCAASGFTLYSSSIHWVRQAPGKGLEWVA
SISSSYGYTSYADSVKGRFTISADTSKNTAYLQMNSLRAEDTAVYYCAR
YRYSSWNRGAIDYWGQGTLVTVSS
AMRA11-24
V_{L}:
(SEQ ID NO: 118)
DIQMTQSPSSLSASVGDRVTITCRASQSVSSAVAWYQQKPGKAPKLLIY
SASSLYSGVPSRFSGSRSGTDFTLTISSLQPEDFATYYCQQSYWWPLTF
GQGTKVEIKRTV
V_{H}:
(SEQ ID NO: 119)
EVQLVESGGGLVQPGGSLRLSCAASGFTVSSSSIHWVRQAPGKGLEWVA
SIYSYYGYTSYADSVKGRFTISADTSKNTAYLQMNSLRAEDTAVYYCAR
WSKSPWYYQGIDYWGQGTLVTVSS

Exemplary Antibody Clones Binding to BTK-Ibrutinib conjugate presented on HLA-A*01:01. CDR residues (Kabat scheme) in bold.

IBA1-4
V_{L}:
(SEQ ID NO: 120)
DIQMTQSPSSLSASVGDRVTITCRASQSVSSAVAWYQQKPGKAPKLLIY
SASSLYSGVPSRFSGSRSGTDFTLTISSLQPEDFATYYCQQYHYWASLI
TFGQGTKVEIKRTV
V_{H}:
(SEQ ID NO: 121)
EVQLVESGGGLVQPGGSLRLSCAASGFTVSSSSIHWVRQAPGKGLEWVA
SIYSYSGSTYYADSVKGRFTISADTSKNTAYLQMNSLRAEDTAVYYCAR
QYSSSYYVWPGMDYWGQGTLVTVSS
IBA1-7
V_{L}:
(SEQ ID NO: 122)
DIQMTQSPSSLSASVGDRVTITCRASQSVSSAVAWYQQKPGKAPKLLIY
SASSLYSGVPSRFSGSRSGTDFTLTISSLQPEDFATYYCQQSYWWKSLV
TFGQGTKVEIKRTV
V_{H}:
(SEQ ID NO: 123)
EVQLVESGGGLVQPGGSLRLSCAASGFTLSSSSIHWVRQAPGKGLEWVA
SISSYYGSTSYADSVKGRFTISADTSKNTAYLQMNSLRAEDTAVYYCAR
MHYSWQEYYSYDWGMDYWGQGTLVTVSS
IBA1-8
V_{L}:
(SEQ ID NO: 124)
DIQMTQSPSSLSASVGDRVTITCRASQSVSSAVAWYQQKPGKAPKLLIY
SASSLYSGVPSRFSGSRSGTDFTLTISSLQPEDFATYYCQQPYYPLITF
GQGTKVEIKRTV
V_{H}:
(SEQ ID NO: 125)
EVQLVESGGGLVQPGGSLRLSCAASGFTISYSSIHWVRQAPGKGLEWVA
SIYPSYGSTSYADSVKGRFTISADTSKNTAYLQMNSLRAEDTAVYYCAR
WQGYYQPALDYWGQGTLVTVSS
IBA1-12
V_{L}:
(SEQ ID NO: 126)
DIQMTQSPSSLSASVGDRVTITCRASQSVSSAVAWYQQKPGKAPKLLIY
SASSLYSGVPSRFSGSRSGTDFTLTISSLQPEDFATYYCQQSSKYYYPI
TFGQGTKVEIKRTV
V_{H}:
(SEQ ID NO: 127)
EVQLVESGGGLVQPGGSLRLSCAASGFTISSSSIHWVRQAPGKGLEWVA
SISPYYGYTSYADSVKGRFTISADTSKNTAYLQMNSLRAEDTAVYYCAR
WGYGWYWYGLDYWGQGTLVTVSS
IBA1-13
V_{L}:
(SEQ ID NO: 128)
DIQMTQSPSSLSASVGDRVTITCRASQSVSSAVAWYQQKPGKAPKLLIY
SASSLYSGVPSRFSGSRSGTDFTLTISSLQPEDFATYYCQQGHDMNPVT
FGQGTKVEIKRTV
V_{H}:
(SEQ ID NO: 129)
EVQLVESGGGLVQPGGSLRLSCAASGFTLSSSSIHWVRQAPGKGLEWVA
SIYSSYGYTYYADSVKGRFTISADTSKNTAYLQMNSLRAEDTAVYYCAR
YYYYWYGGMDYWGQGTLVTVSS
IBA1-19
V_{L}:
(SEQ ID NO: 130)
DIQMTQSPSSLSASVGDRVTITCRASQSVSSAVAWYQQKPGKAPKLLIY
SASSLYSGVPSRFSGSRSGTDFTLTISSLQPEDFATYYCQQSWMSDSLI
TFGQGTKVEIKRTV
V_{H}:
(SEQ ID NO: 131)
EVQLVESGGGLVQPGGSLRLSCAASGFTFSYSSIHWVRQAPGKGLEWVA
SIYPSSGYTSYADSVKGRFTISADTSKNTAYLQMNSLRAEDTAVYYCAR
GWWYWMAWDYAMDYWGQGTLVTVSS
IBA1-21
V_{L}:
(SEQ ID NO: 132)
DIQMTQSPSSLSASVGDRVTITCRASQSVSSAVAWYQQKPGKAPKLLIY
SASSLYSGVPSRFSGSRSGTDFTLTISSLQPEDFATYYCQQMQYSGWLI
TFGQGTKVEIKRTV
V_{H}:
(SEQ ID NO: 133)
EVQLVESGGGLVQPGGSLRLSCAASGFTISSSSIHWVRQAPGKGLEWVA
SISSYYGYTSYADSVKGRFTISADTSKNTAYLQMNSLRAEDTAVYYCAR
YYSYSSGYGYYDYFDWGAMDYWGQGTLVTVSS

Exemplary Antibody Clones binding to EGFR-Osimertinib conjugate presented on HLA-A*02:01. CDR residues (Kabat scheme) in bold.

OEA2-1
V_{L}:
(SEQ ID NO: 134)
DIQMTQSPSSLSASVGDRVTITCRASQSVSSAVAWYQQKPGKAPKLLIY
SASSLYSGVPSRFSGSRSGTDFTLTISSLQPEDFATYYCQQSSSSLITF
GQGTKVEIKRTV
V_{H}:
(SEQ ID NO: 135)
EVQLVESGGGLVQPGGSLRLSCAASGFTFSSSSIHWVRQAPGKGLEWVA
SISSSSGSTSYADSVKGRFTISADTSKNTAYLQMNSLRAEDTAVYYCAR
YYGYVWGGYWGWWYSKALDYWGQGTLVTVSS
OEA2-5
V_{L}:
(SEQ ID NO: 5)
DIQMTQSPSSLSASVGDRVTITCRASQSVSSAVAWYQQKPGKAPKLLIY
SASSLYSGVPSRFSGSRSGTDFTLTISSLQPEDFATYYCQQYSYWPITF
GQGTKVEIKRTV
V_{H}:
(SEQ ID NO: 6)
EVQLVESGGGLVQPGGSLRLSCAASGFTISSSYIHWVRQAPGKGLEWVA
YISPSYGSTSYADSVKGRFTISADTSKNTAYLQMNSLRAEDTAVYYCAR
EYVTMALDYWGQGTLVTVSS
OEA2-12
V_{L}:
(SEQ ID NO: 136)
DIQMTQSPSSLSASVGDRVTITCRASQSVSSAVAWYQQKPGKAPKLLIY
SASSLYSGVPSRFSGSRSGTDFTLTISSLQPEDFATYYCQQYDWNYYLV
TFGQGTKVEIKRTV
V_{H}:
(SEQ ID NO: 137)
EVQLVESGGGLVQPGGSLRLSCAASGFTIYSSSIHWVRQAPGKGLEWVA
SISSYYGYTSYADSVKGRFTISADTSKNTAYLQMNSLRAEDTAVYYCAR
YQYYGSLYYSQQWAMDYWGQGTLVTVSS
OEA2-16
V_{L}:
(SEQ ID NO: 138)
DIQMTQSPSSLSASVGDRVTITCRASQSVSSAVAWYQQKPGKAPKLLIY
SASSLYSGVPSRFSGSRSGTDFTLTISSLQPEDFATYYCQQSSSSLITF
GQGTKVEIKRTV
V_{H}:
(SEQ ID NO: 139)
EVQLVESGGGLVQPGGSLRLSCAASGFTFSSSSIHWVRQAPGKGLEWVA
SISSSSGSTSYADSVKGRFTISADTSKNTAYLQMNSLRAEDTAVYYCAR
SPSSPYFMSWGWYWQYGIDYWGQGTLVTVSS
OEA2-21
V_{L}:
(SEQ ID NO: 140)
DIQMTQSPSSLSASVGDRVTITCRASQSVSSAVAWYQQKPGKAPKLLIY
SASSLYSGVPSRFSGSRSGTDFTLTISSLQPEDFATYYCQQSSWGGLVT
FGQGTKVEIKRTV
V_{H}:
(SEQ ID NO: 141)
EVQLVESGGGLVQPGGSLRLSCAASGFTFSSSYIHWVRQAPGKGLEWVA
SISPSYGYTSYADSVKGRFTISADTSKNTAYLQMNSLRAEDTAVYYCAR
DMYEWWHWAIDYWGQGTLVTVSS
OEA2-24
V_{L}:
(SEQ ID NO: 142)
DIQMTQSPSSLSASVGDRVTITCRASQSVSSAVAWYQQKPGKAPKLLIY
SASSLYSGVPSRFSGSRSGTDFTLTISSLQPEDFATYYCQQSSSSLITF
GQGTKVEIKRTV
V_{H}:
(SEQ ID NO: 143)
EVQLVESGGGLVQPGGSLRLSCAASGFTFSSSSIHWVRQAPGKGLEWVA
SISSSSGSTSYADSVKGRFTISADTSKNTAYLQMNSLRAEDTAVYYCAR
YGHYLYYWGWGWYWSAALDYWGQGTLVTVSS

### REFERENCES RELATED TO EXAMPLE 3

1. Miller K R, Koide A, Leung B, Fitzsimmons J, Yoder B, Yuan H, Jay M, Sidhu S S, Koide S, Collins E J. T cell receptor-like recognition of tumor in vivo by synthetic antibody fragment. PloS one. 2012; 7(8):e43746. Epub 2012 Aug. 24. doi: 10.1371/journal.pone.0043746. PubMed PMID: 22916301; PMCID: 3423377.
2. Hattori T, Koide A, Panchenko T, Romero L A, Teng K W, Corrado A D, Koide S. Multiplex bead binding assays using off-the-shelf components and common flow cytometers. J Immunol Methods. 2020:112952. Epub 2020 Dec. 29. doi: 10.1016/j.jim.2020.112952. PubMed PMID: 33358997.

### Example 4

This Example demonstrates single-chain Diabody (scDb) formats of Hapimmune antibodies and their effectiveness in cell killing. Data from non-limiting embodiments are presented in FIGS. 18 and 19. The results are summarized as in the brief descriptions of FIGS. 18 and 19.

To obtain the results for FIG. 18, Raji cells and T2 cells (ATCC) were cultured in RPMI supplemented with 10% fetal bovine serum (FBS) and penicillin/streptomycin. The cytotoxic effect of scDbs was measured by following the protocol published previously (ref: PMID 26813960). Briefly, Raji cells or T2 cells were stained with carboxyfluorescein succinimidyl ester (CSFE, ThermoFisher, 65-0850-84), then incubated with the final 10 μM KRAS(G12C)-AMG510 conjugate or 1 μM EGFR-Osimertinib in the presence of 10 μg/mL human beta-2 microglobulin for 4 hr. The cells were harvested using centrifugation and washed in media to remove the unbound conjugate and peptide. Peptide-drug-pulsed cells were then co-cultured with human T-cells (E:T=3:1) in the presence of single-chain Diabodies (scDbs) for 19-21 hr. After incubation, cells were harvested and washed with PBS, then stained with Fixable Viability Dye eFluor660 (ThermoFisher, 65-0864-14). After washing cells, the cells were analyzed on iQue screener (Sartorius).

To obtain the results for FIG. 19, lung cancer cell lines were cultured in RPMI supplemented with 10% fetal bovine serum (FBS) and penicillin/streptomycin. For cytotoxicity assays, 1×10⁴ cells/well were seeded in 96-well flat bottom plates and incubated at 37° C., 5% CO₂ for 24 hours. Media were replaced with fresh media supplemented with 100 nM AMG510 or DMSO, then the cells were incubated for 24 hours at 37° C. After incubation, cells were co-cultured with human T cells (E:T=5:1) and AMRA3-7_UCHT1 scDb in the presence of 100 IU/mL IL-2 for 24 hr at 37° C. Cell viability was assessed by using PrestoBlue™ Cell Viability Reagent (ThermoFisher, A13261). Cytotoxicity was calculated by taking the fluorescent signal of a given well, subtracting the fluorescent signal from the wells that contain only T-cells, and normalizing to the fluorescent signal from the wells without scDb.

### Exemplary Single-Chain Diabody Clones Targeting Both HLA-A*03:01 RAS-AMG510 and HLA-A*11:01_RAS-AMG510

The italicized sequences represent AviTag and HisTag, respectively.

AMRA3-7_UCHT1_scDb
(SEQ ID NO: 144, sequence without tag disclosed as SEQ ID NO: 161)
DIVRSDIQMTQSPSSLSASVGDRVTITCRASQSVSSAVAWYQQKPGKAPKLLI
YSASSLYSGVPSRFSGSRSGTDFTLTISSLQPEDFATYYCQQISYVYSLITFG
QGTKVEIKGGGGSEVQLQQSGPELVKPGASMKISCKASGYSFTGYTMNWVKQS
HGKNLEWMGLINPYKGVSTYNQKFKDKATLTVDKSSSTAYMELLSLTSEDS
AVYYCARSGYYGDSDWYFDVWGQGTTLTVSSGGGGSGGGGSGGGGSDIQM
TQTTSSLSASLGDRVTISCRASQDIRNYLNWYQQKPDGTVKLLIYYTSRLHSG
VPSKFSGSGSGTDYSLTISNLEQEDIATYFCQQGNTLPWTFAGGTKLEIKGGG
GSEVQLVESGGGLVQPGGSLRLSCAASGFTFSSYSIHWVRQAPGKGLEWVASI
YSSYGYTSYADSVKGRFTISADTSKNTAYLQMNSLRAEDTAVYYCARGGWY
PAMDYWGQGTLVTVSSLEGGGGLNDIFEAQKIEWHESRHHHHHH
AMRA311-16_UCHT1_scDb
(SEQ ID NO: 145, sequence without tag disclosed as SEQ ID NO: 162)
DIVRSDIQMTQSPSSLSASVGDRVTITCRASQSVSSAVAWYQQKPGKAPKLLI
YSASSLYSGVPSRFSGSRSGTDFTLTISSLQPEDFATYYCQQSSSSLITFGQG
TKVEIKGGGGSEVQLQQSGPELVKPGASMKISCKASGYSFTGYTMNWVKQSHG
KNLEWMGLINPYKGVSTYNQKFKDKATLTVDKSSSTAYMELLSLTSEDSAV
YYCARSGYYGDSDWYFDVWGQGTTLTVSSGGGGSGGGGSGGGGSDIQMTQ
TTSSLSASLGDRVTISCRASQDIRNYLNWYQQKPDGTVKLLIYYTSRLHSGVP
SKFSGSGSGTDYSLTISNLEQEDIATYFCQQGNTLPWTFAGGTKLEIKGGGGS
EVQLVESGGGLVQPGGSLRLSCAASGFTISSSSIHWVRQAPGKGLEWVAYIS
SYSGYTYYADSVKGRFTISADTSKNTAYLQMNSLRAEDTAVYYCARYWYGHY
HSYFGLDYWGQGTLVTVSSLEGGGGLNDIFEAQKIEWHESRHHHHHH

### Exemplary Single-Chain Diabody Clones Targeting HLA-A*02:01_EGFR-Osimertinib

The italicized sequences represent AviTag and HisTag, respectively.

OEA2-5_UCHT1_scDb
(SEQ ID NO: 146, sequence without tag disclosed as SEQ ID NO: 163)
DIVRSDIQMTQSPSSLSASVGDRVTITCRASQSVSSAVAWYQQKPGKAPKLLI
YSASSLYSGVPSRFSGSRSGTDFTLTISSLQPEDFATYYCQQYSYWPITFGQGT
KVEIKGGGGSEVQLQQSGPELVKPGASMKISCKASGYSFTGYTMNWVKQSH
GKNLEWMGLINPYKGVSTYNQKFKDKATLTVDKSSSTAYMELLSLTSEDSA
VYYCARSGYYGDSDWYFDVWGQGTTLTVSSGGGGSGGGGSGGGGSDIQMT
QTTSSLSASLGDRVTISCRASQDIRNYLNWYQQKPDGTVKLLIYYTSRLHSGV
PSKFSGSGSGTDYSLTISNLEQEDIATYFCQQGNTLPWTFAGGTKLEIKGGGGS
EVQLVESGGGLVQPGGSLRLSCAASGFTISSSYIHWVRQAPGKGLEWVAYISP
SYGSTSYADSVKGRFTISADTSKNTAYLQMNSLRAEDTAVYYCAREYVTMA
LDYWGQGTLVTVSSLEGGGGLNDIFEAQKIEWHESRHHHHHH
OEA2-21_UCHT1_scDb
(SEQ ID NO: 147, sequence without tag disclosed as SEQ ID NO: 164)
DIVRSDIQMTQSPSSLSASVGDRVTITCRASQSVSSAVAWYQQKPGKAPKLLI
YSASSLYSGVPSRFSGSRSGTDFTLTISSLQPEDFATYYCQQSSWGGLVTFGQG
TKVEIKGGGGSEVQLQQSGPELVKPGASMKISCKASGYSFTGYTMNWVKQS
HGKNLEWMGLINPYKGVSTYNQKFKDKATLTVDKSSSTAYMELLSLTSEDS
AVYYCARSGYYGDSDWYFDVWGQGTTLTVSSGGGGSGGGGSGGGGSDIQM
TQTTSSLSASLGDRVTISCRASQDIRNYLNWYQQKPDGTVKLLIYYTSRLHSG
VPSKFSGSGSGTDYSLTISNLEQEDIATYFCQQGNTLPWTFAGGTKLEIKGGG
GSEVQLVESGGGLVQPGGSLRLSCAASGFTFSSSYIHWVRQAPGKGLEWVASI
SPSYGYTSYADSVKGRFTISADTSKNTAYLQMNSLRAEDTAVYYCARDMYE
WWHWAIDYWGQGTLVTVSSLEGGGGLNDIFEAQKIEWHESRHHHHHH

### Example 5

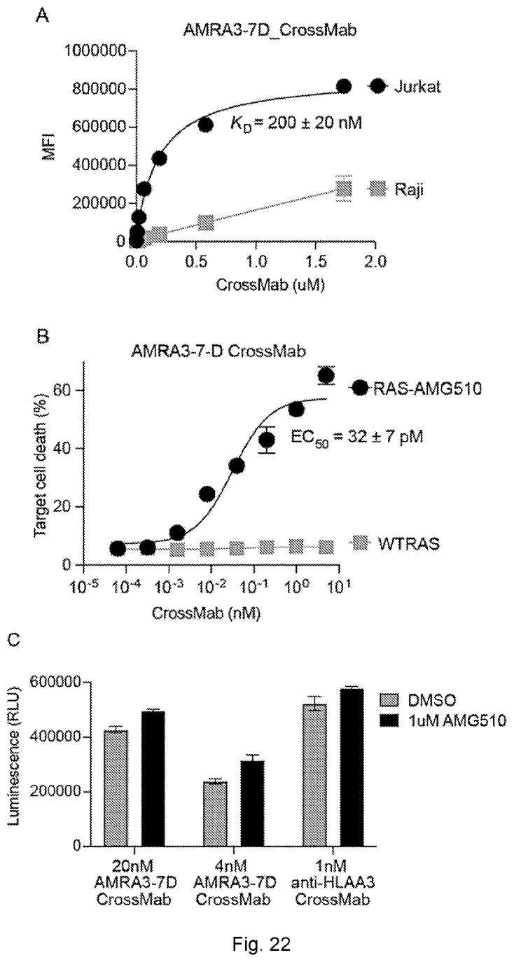

This Example demonstrates scDb and 2+1 CrossMab antibodies constructed with the AMRA3-7 clone and their effectiveness in cell killing. Data from non-limiting embodiments are presented in FIGS. 21-22.

To obtain the results for FIG. 21 (A) and FIG. 22 (B), Raji cells (ATCC) were cultured in RPMI supplemented with 10% fetal bovine serum (FBS) and penicillin/streptomycin. The cytotoxic effects of scDb and CrossMab were measured by following the protocol published previously (ref: PMID 26813960). Briefly, Raji cells were stained with carboxyfluorescein succinimidyl ester (CSFE, ThermoFisher, 65-0850-84), then incubated with 10 μM KRAS(G12C)-AMG510 conjugate or 10 μM KRAS(WT) peptide (final concentrations) in the presence of 10 μg/mL human beta-2 microglobulin for 4 hr. The cells were harvested by centrifugation and washed in media to remove the unbound conjugate and peptide. Peptide-drug-pulsed cells were then co-cultured with human T-cells (E:T=3:1) in the presence of scDb or CrossMab for 19 hr. After incubation, cells were harvested and washed with PBS, then stained with Fixable Viability Dye eFluor660 (ThermoFisher, 65-0864-14). After washing again, cells were analyzed on iQue screener (Satorius).

To obtain the results for FIGS. 21 (B) and 22 (C), H2122 cells (ATCC) were cultured in RPMI supplemented with 10% fetal bovine serum (FBS) and penicillin/streptomycin. For cytotoxicity assays, 5×10³ cells/well were seeded in 96-well flat bottom plates in the presence of 1 mM AMG510 or DMSO and 5 μg/mL human beta-2 microglobulin, and then were incubated at 37° C., 5% CO₂ for 48 hours. After incubation, cells were co-cultured with human T cells (E:T=10:1) and AMRA3-7D scDb or CrossMab in the presence of 10 ng/mL IL7 and IL15 for 24 hr at 37° C. Dead cells were measured by using CytoTox-Glo cytotoxic assay (Promega, G9290). The luminescent signal of a given well was calculated by subtracting the signal from wells that contain H2122 and T-cells without scDb or CrossMab constructs.

To obtain the results for FIG. 22 (A), Jurkat and Raji cells (ATCC) were cultured in RPMI supplemented with 10% fetal bovine serum (FBS) and penicillin/streptomycin. Cells were washed twice with PBS, then incubated with AMRA3-7D CrossMab at 4° C. for 30 min. After washing three times with PBS containing 1% BSA (PBS/BSA), cells were stained with Alexa647 Goat Anti-Human IgG Fc (Jackson ImmunoResearch, 109-605-098). After incubation, cells were washed three times with PBS/BSA and analyzed on iQue screener (Sartorius).

AMRA3-7D_UCHT1_scDb
(SEQ ID NO: 148, sequence without tag disclosed as SEQ ID NO: 165)
DIVRSDIQMTQSPSSLSASVGDRVTITCRASQSVSSAVAWYQQKPGKAPKLLI
YSASSLYSGVPSRFSGSRSGTDFTLTISSLQPEDFATYYCQQISYVKKLITFGQ
GTKVEIKGGGGSEVQLQQSGPELVKPGASMKISCKASGYSFTGYTMNWVKQ
SHGKNLEWMGLINPYKGVSTYNQKFKDKATLTVDKSSSTAYMELLSLTSEDS
AVYYCARSGYYGDSDWYFDVWGQGTTLTVSSGGGGSGGGGSGGGGSDIQM
TQTTSSLSASLGDRVTISCRASQDIRNYLNWYQQKPDGTVKLLIYYTSRLHSG
VPSKFSGSGSGTDYSLTISNLEQEDIATYFCQQGNTLPWTFAGGTKLEIKGGG
GSEVQLVESGGGLVQPGGSLRLSCAASGFTFSDYSIHWVRQAPGKGLEWVAS
ISSSSGSTSYADSVKGRFTISADTSKNTAYLQMNSLRAEDTAVYYCARGGW
YPAMDYWGQGTLVTVSSLEGGGGLNDIFEAQKIEWHESRHHHHHH
AMRA3-7D_CrossMab
>Chain A. QMY30735.1
(SEQ ID NO: 149)
EVQLLESGGGLVQPGGSLRLSCAASGFTFSTYAMNWVRQAPGKGLEWVSRIR
SKYNNYATYYADSVKGRFTISRDDSKNTLYLQMNSLRAEDTAVYYCVRHGN
FGNSYVSWFAYWGQGTLVTVSSASVAAPSVFIFPPSDEQLKSGTASVVCLLN
NFYPREAKVQWKVDNALQSGNSQESVTEQDSKDSTYSLSSTLTLSKADYEKH
KVY
ACEVTHQGLSSPVTKSFNRGEC
>Chain B.
(SEQ ID NO: 150)
EVQLVESGGGLVQPGGSLRLSCAASGFTFSDYSIHWVRQAPGKGLEWVASISS
SSGSTSYADSVKGRFTISADTSKNTAYLQMNSLRAEDTAVYYCARGGWYPA
MDYWGQGTLVTVSSASTKGPSVFPLAPSSKSTSGGTAALGCLVEDYFPEPVT
VSWNSGALTSGVHTFPAVLQSSGLYSLSSVVTVPSSSLGTQTYICNVNHKPSN
TKVDEKVEPKSCDGGGGSGGGGSQAVVTQEPSLTVSPGGTVTLTCGSSTGAV
TTSNYANWVQEKPGQAFRGLIGGTNKRAPGTPARFSGSLLGGKAALTLSGAQ
PEDEAEYYCALWYSNLWVFGGGTKLTVLSSASTKGPSVFPLAPSSKSTSGGT
AALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPEAAGGPSVFLFPP
KPKDTLMISRTPEVTCVVVDVSHEDPEVKFNWYVDGVEVHNAKTKPREEQY
NSTYRVVSVLTVLHQDWLNGKEYKCKVSNKALGAPIEKTISKAKGQPREPQV
YTLPPCRDELTKNQVSLWCLVKGFYPSDIAVEWESNGQPENNYKTTPPVLDS
DGSFFLYSKLTVDKSRWQQGNVFSCSVMHEALHNHYTQKSLSLSP
>Chain C.
(SEQ ID NO: 151)
EVQLVESGGGLVQPGGSLRLSCAASGFTFSDYSIHWVRQAPGKGLEWVASISS
SSGSTSYADSVKGRFTISADTSKNTAYLQMNSLRAEDTAVYYCARGGWYPA
MDYWGQGTLVTVSSASTKGPSVFPLAPSSKSTSGGTAALGCLVEDYFPEPVT
VSWNSGALTSGVHTFPAVLQSSGLYSLSSVVTVPSSSLGTQTYICNVNHKPSN
TKVDEKVEPKSCDKTHTCPPCPAPEAAGGPSVFLFPPKPKDTLMISRTPEVTC
VVVDVSHEDPEVKFNWYVDGVEVHNAKTKPREEQYNSTYRVVSVLTVLHQ
DWLNGKEYKCKVSNKALGAPIEKTISKAKGQPREPQVCTLPPSRDELTKNQV
SLSCAVKGFYPSDIAVEWESNGQPENNYKTTPPVLDSDGSFFLVSKLTVDKSR
WQQGNVFSCSVMHEALHNHYTQKSLSLSP
>Chain D.
(SEQ ID NO: 152)
DIQMTQSPSSLSASVGDRVTITCRASQSVSSAVAWYQQKPGKAPKLLIYSASS
LYSGVPSRFSGSRSGTDFTLTISSLQPEDFATYYCQQISYVKKLITFGQGTKV
EIKRTVAAPSVFIFPPSDRKLKSGTASVVCLLNNFYPREAKVQWKVDNALQSG
NSQESVTEQDSKDSTYSLSSTLTLSKADYEKHKVYACEVTHQGLSSPVTKSFN
RGEC

FIG. 21 shows the cytotoxic effect of AMRA3-7D in the single-chain Diabody (scDb) format. (A) Raji cells were first pulsed with AMG510 conjugated to a peptide corresponding to a fragment of KRAS(G12C) or a control peptide corresponding to KRAS(wild type). Pulsed cells were co-cultured with human T cells (Effector:Target=3:1) in the presence of scDb at the indicated concentrations. After incubation for 17 hours, dead cells were stained and detected by flow cytometry. (B) H2122 cells were first incubated with AMG510 or DMSO. The cells were then co-cultured with human T cells (Effector:Target=10:1) in the presence of scDb at the indicated concentrations. After incubation, dead cells were measured by detecting a distinct intracellular protease activity released from membrane-compromised cells. Data shown here are from triplicate measurements. Error bars indicate the s.d. Where error bars are not visible, the errors are smaller than the symbols. Anti-HLA-A3 is a positive control.

FIG. 22 shows the CD3 binding properties and cytotoxic effects of AMRA3-7D in the CrossMab format. (A) Binding titration curve of AMRA3-7 CrossMab to Jurkat (CD3 positive) and Raji (CD3 negative) cells. The apparent K_{D} value is shown. Note that cells were not pulsed with any peptides. (B, C) Cytotoxic effects of AMRA3-7D CrossMab on Raji cells pulsed with an exogenous peptide-drug conjugate (B) and on H2122 cells treated with the drug (C). Methods are the same as in FIG. 22. Data shown here are from triplicate measurements. Error bars indicate the s.d. Where error bars are not visible, the errors are smaller than the symbols. Anti-HLA-A3 serves as a positive control.

### Example 6

This example demonstrates deep mutational analysis of the AMR-A3-7D and OEA2-5 antibodies. Data from non-limiting embodiments is presented in FIGS. 23 and 24.

To identify mutations in CDRs of AMR-A3-7D and OEA2-5 that retain antigen binding, we performed deep mutational scanning on residues CDR-L3 and CDR-H3. In the yeast display format, each of the CDR-L3 and CDR-H3 residues were mutated to all genetically encoded amino acids using the NNK codon (N=A, T, G and C; K=G and T), one residue at a time. The resulting pool of mutants was combined, and the library was subjected to FACS using the relevant antigen, i.e., AMG510-KRAS(G12C) peptide in complex with HLA-A*03:01 for AMR-A3-7D and Osimertinib-EGFR in complex with HLA-A*02:01. We used different antigen concentrations in order to adjust the stringency of library sorting. Vectors recovered from binding-capable and binding-incapable pools were analyzed by deep sequencing on an Illumina MiSeq instrument. Mutations found in different pools were deduced from the DNA sequencing analysis.

From this analysis, the disclosure provides the following permissible mutations at each CDR position as shown in the tables below. As references, the VL and VH sequences of the parent clones are shown, with the analyzed CDR residues in bold an italics. In embodiments, the disclosure includes each mutation alone, and all combination of mutations. Thus, as evident from the Tables, the disclosure included the described CDRs with 1, 2, 3, 4, 5, 6, 7, or 8 mutations as indicated in the Tables. The disclosure includes additional amino acid chances, such as in CDR1, CDR2, and in the framework sequences.

AMRA3-7D
V_{L}:
(SEQ ID NO: 3)
DIQMTQSPSS LSASVGDRVT ITCRASQSVS SAVAWYQQKP
GKAPKLLIYS ASSLYSGVPS RFSGSRSGTD FTLTISSLQP
EDFATYYCQQ ISYVKKLITF GQGTKVEIKR TV
V_{H}:
(SEQ ID NO: 4)
EVQLVESGGG LVQPGGSLRL SCAASGFTFS DYSIHWVRQA
PGKGLEWVAS ISSSSGSTSY ADSVKGRFTI SADTSKNTAY
LQMNSLRAED TAVYYCARGG WYPAMDYWGQ GTLVTVSS

TABLE A
Parental and consensus sequences disclosed
as SEQ ID NOS 153-154, respectively

| Position | Parental amino | | Consensus amino |
|---|---|---|---|
| (VL) | acid residue | Permissible mutation | acid residue |
| 91 | I | A, L, P, S, T, V | A, I, L, P, S, T, V |
| 92 | S | K, R, T | K, R, S, T |
| 93 | Y | F | F, Y |
| 94 | V | I, R, T | I, R, T, V |
| 95 | K | A, E, H, L, M, N, | A, E, H, K, L, M, N, |
| | | Q, R, S, T, Y | Q, R, S, T, Y |
| 96 | K | R | K, R |
| 97 | L | A, C, D, E, G, H, | A, C, D, E, G, H, K, |
| | | K, M, N, P, Q, R, | L, M, N, P, Q, R, S, |
| | | S, T, V, W | T, V, W |
| 98 | I | L, V | I, L, V |

TABLE B
Parental and consensus sequences disclosed
as SEQ ID NOS 155-156, respectively

| | Parental | | |
|---|---|---|---|
| Position | amino acid | | Consensus amino |
| (VH) | residue | Permissible mutation | acid residue |
| 99 | G | | G |
| 100 | G | A, C, E, H, K, L, M, | A, C, E, G, H, K, L, |
| | | N, P, Q, R, S, T, W, Y | M, N, P, Q, R, S, T, |
| | | | W, Y |
| 101 | W | G, P, R, T | G, P, R, T, W |
| 102 | Y | A, D, E, F, G, H, I, | A, D, E, F, G, H, I, |
| | | K, M, N, Q, R, S, T, W | K, M, N, Q, R, S, T, |
| | | | W, Y |
| 103 | P | A, V | A, P, V |
| 104 | A | C, G, K, L, M, Q, R, S, | A, C, G, K, L, M, Q, |
| | | T, Y | R, S, T, Y |

### Deep Mutational Scanning of OEA2-5

### OEA2-5

V_{L}:
(SEQ ID NO: 5)
DIQMTQSPSS LSASVGDRVT ITCRASQSVS SAVAWYQQKP
GKAPKLLIYS ASSLYSGVPS RFSGSRSGTD FTLTISSLQP
EDFATYYCQQ YSYWPITFGQ GTKVEIKRTV
V_{H}:
(SEQ ID NO: 6)
EVQLVESGGG LVQPGGSLRL SCAASGFTIS SSYIHWVRQA
PGKGLEWVAY ISPSYGSTSY ADSVKGRFTI SADTSKNTAY
LQMNSLRAED TAVYYCAREY VTMALDYWGQ GTLVTVSS

TABLE C
Parental and consensus sequences disclosed
as SEQ ID NOS 157-158, respectively

| | Parental | | |
|---|---|---|---|
| Position | amino acid | | Consensus amino |
| (VH) | residue | Permissible mutation | acid residue |
| 91 | Y | | Y |
| 92 | S | A | A, S |
| 93 | Y | A, C, D, E, F, G, H, I, | A, C, D, E, F, G, H, |
| | | K, L, M, N, Q, R, S, T, | I, K, L, M, N, Q, R, |
| | | V, W | S, T, V, W, Y |
| 94 | W | | W |
| 95 | P | | P |
| 96 | I | E, P | E, I, P |

TABLE D
Parental and consensus sequences disclosed
as SEQ ID NOS 159-160, respectively

| Position | Parental amino | | Consensus amino |
|---|---|---|---|
| (VH) | acid residue | Permissible mutation | acid residue |
| 99 | E | D | D, E |
| 100 | Y | E, F, L, Q, S, W | E, F, L, Q, S, W, Y |
| 101 | V | T, I | I, T, V |
| 102 | T | A, E, N, S | A, E, N, S, T |
| 103 | M | F, Y | F, M, Y |
| 104 | A | C, G, S, T | A, C, G, S, T |
| 105 | L | A, C, E, F, H, I, K, | A, C, E, F, H, I, K, |
| | | M, N, P, Q, S, T, V | L, M, N, P, Q, S, T, V |

As will be evident from the foregoing tables, in one embodiment, the binding partner comprises a light chain that comprises a complementary determining region 3 (CDR3) that comprises the sequence SEQ ID NO:154 and a heavy chain that comprises a CDR3 that comprises the sequence of SEQ ID NO:156. In another embodiment, the binding partner the binding partner comprises a light chain that comprises a CDR3 that comprises the sequence of SEQ ID NO:158 and a heavy chain that comprises a CDR3 that comprises the sequence of SEQ ID NO:160.

FIG. 23 describes deep mutational scanning analysis of the CDR-L3 and H3 residues of AMR-A3-7D. (a) Representative flow cytometry profiles of yeast cells displaying AMRA3-7D or its deep mutational scanning library populations. Binding to 5 nM HLA-A*03:01 presenting AMG510 conjugated to the Cys residue in the peptides, VVGACGVGK (SEQ ID NO: 1), was measured. The profile of the parental antibody, AMRA3-7D, is shown on the left, and those for sorted subsets of the deep mutational scanning library are shown on the right. The library was sorted with 1, 3, and 10 nM target, referred to as conditions 1, 2 and 3, respectively, and the nonbinder pool was sorted with 50 nM target. (b) The prevalence of mutations in the sorted subsets of the deep mutational scanning library is presented in a heat map format. The number of deep sequencing reads were normalized to the total reads for each sorted pool and multiplied by 1000. Crosses indicate the wild-type amino acid.

FIG. 24 describes results from deep mutational scanning analysis of CDR-L3 and H3 residues of OEA2-5. (a) Representative flow cytometry profiles of yeast cells displaying OEA2-5 in single-chain Fv format and its deep mutational scanning library populations. Binding to 1.5 nM streptavidin tetramer saturated with biotinylated HLA-A*02:01 presenting Osimertinib conjugated to the Cys residue in the peptides, QLMPFGCLL (SEQ ID NO: 30), was measured. The profile of the parental antibody, OEA2-5, is shown on the left, and those for sorted subsets of the deep mutational scanning library are shown on the right. The library was sorted with 12.5, 2.5, and 0.5 nM target, referred to as conditions 1, 2 and 3, respectively, and the nonbinder pool was sorted with 12.5 nM target. (b) The prevalence of mutations in the sorted subsets of the deep mutational scanning library is presented in a heat map format. The number of deep sequencing reads were normalized to the total reads for each sorted pool and multiplied by 1000. Crosses indicate the wild-type amino acid.

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A binding partner that binds with specificity to a site on a protein or peptide that comprises a covalently linked molecule (a peptide conjugate).

2. The binding partner of claim 1, wherein the binding partner does not bind, or binds with a lower affinity, to a control protein or control peptide that does not comprise the covalently linked molecule.

3. The binding partner of claim 1, wherein the binding partner binds with specificity to the peptide conjugate, said peptide conjugate being in a complex with a major histocompatibility complex (MHC).

4. The binding partner of claim 3, wherein the MHC is a human leukocyte antigen (HLA).

5. The binding partner of claim 1, wherein the site on the peptide conjugate comprises a nucleophilic or an electrophilic residue, said residue optionally being one of Cys, Lys, Tyr, or His.

6. The binding partner of claim 5, wherein the site comprises the Cys.

7. The binding partner of claim 6, wherein the molecule is covalently linked to the Cys.

8. The binding partner of claim 1, wherein the site is present as a peptide conjugate in an intact protein, and wherein optionally the intact protein is in its native confirmation.

9. The binding partner of any one of claims 1-8, wherein the binding partner is comprised by an intact antibody, an antigen-binding (Fab) fragment, an Fab′ fragment, an (Fab′)2 fragment, an Fd, an Fv, a dAb, a single domain fragment or single monomeric variable antibody domain, a single-chain Diabody (scDb), a single-chain variable fragment (scFv), a Bi-specific T-cell engager (BiTE), bispecific killer cell engager (BiKE), CrossMab, a tri-specific binding partner, a or chimeric antigen receptor (CAR).

10. The binding partner of claim 9, wherein the binding partner is conjugated to a detectable label, or a chemotherapeutic agent, a radioisotope, or a toxin.

11. The binding partner of claim 9, wherein the binding partner is a component of a fusion protein.

12. The binding partner of claim 9, wherein the peptide conjugate is comprised by a segment of a protein that is associated with a cancer.

13. The binding partner of claim 9, wherein the peptide conjugate is comprised by a protein that has enzymatic activity, and wherein the covalently linked molecule is an inhibitor of said protein that has the enzymatic activity.

14. The binding partner of claim 13, wherein the protein that has the enzymatic activity is a kinase or a GTPase.

15. The binding partner of claim 9, wherein the peptide conjugate comprises a mutation, and wherein peptide component of the peptide conjugate is or is derived from Bruton's tyrosine kinase (BTK), any epidermal growth factor receptor (EGFR) family member that is selected from EGFR (ERBB1), HER2/NEU (ERBB2), HER3 (ERBB3), and HER4 (ERBB4); MET (HGFR); any fibroblast growth factor receptor (FGFR); any cyclin-dependent kinase (CDK); Acetylcholine Esterase (ACHE); p90 ribosomal S6 kinase (RSK); TP53, IDH1, GNAS, FBXW7, CTNNB1, DNMT3A, a cathepsin that is selected from cathepsin B, C, F, H, K, L, O, S, V, W and X; any caspase; and a protein involved in obesity that is optionally Pancreatic lipase or METAP2.

16. The binding partner of claim 9, wherein the peptide conjugate comprises a KRAS^{G12C} mutation.

17. The binding partner of claim 9, wherein the peptide conjugate comprises the KRAS^{G12C} mutation, and the drug comprises a G12C inhibitor.

18. The binding partner of claim 9, wherein the peptide conjugate comprises a fragment of EGFR and the drug comprises an inhibitor of an EGFR family kinase.

19. The binding partner of claim 9, wherein the molecule is one of: AMG510, Osimertinib, ARS-1620, MRTX849, JNJ74699157, LY3499446, MRTX-1275, or a G12C degrader.

20. The binding partner of claim 9, wherein the binding partner specifically binds to a KRAS(G12C)-AMG510 peptide conjugate or said peptide conjugate presented on HLA-A*03:01 and/or HLA-A*11:01.

21. The binding partner of claim 9, wherein the binding agent specifically binds to a peptide conjugate that is BTK-Ibrutinib that is optionally presented on HLA-A*01:01.

22. The binding partner of claim 9, wherein the binding agent specifically binds to a peptide conjugate that is an epidermal growth factor receptor (EGFR)-Osimertinib conjugate that is optionally presented on HLA-A*02:01.

23. The binding partner of claim 9, wherein the binding partner comprises at least one heavy chain (V_{H}), or at least one pair of a light chain (V_{L}) and a heavy chain that are each at least 90% identical to heavy and light chain pairs that comprise the sequences of:
AMRA3-7D:
V_{L}:
(SEQ ID NO: 3)
DIQMTQSPSSLSASVGDRVTITCRASQSVSSAVAWYQQKPGKAPKLLIYSASSLYS
GVPSRFSGSRSGTDFTLTISSLQPEDFATYYCQQISYVKKLITFGQGTKVEIKRTV
V_{H}:
(SEQ ID NO: 4)
EVQLVESGGGLVQPGGSLRLSCAASGFTFSDYSIHWVRQAPGKGLEWVASISSSS
GSTSYADSVKGRFTISADTSKNTAYLQMNSLRAEDTAVYYCARGGWYPAMDYWG
QGTLVTVSS;
OEA2-5:
V_{L}:
(SEQ ID NO: 5)
DIQMTQSPSSLSASVGDRVTITCRASQSVSSAVAWYQQKPGKAPKLLIYSASSLYS
GVPSRFSGSRSGTDFTLTISSLQPEDFATYYCQQYSYWPITFGQGTKVEIKRTV
V_{H}:
(SEQ ID NO: 6)
EVQLVESGGGLVQPGGSLRLSCAASGFTISSSYIHWVRQAPGKGLEWVAYISPSY
GSTSYADSVKGRFTISADTSKNTAYLQMNSLRAEDTAVYYCAREYVTMALDYWGQ
GTLVTVSS
P2AMR-1
V_{L}:
(SEQ ID NO: 24)
QSVLIQPRSVSGSPGQSVTISCTGTSSDVGGYNYVSWYQQHPGKAPKLMIYDVSK
RPSGVPDRFSGSKSGNTASLTVSGLQAEDEADYYCGSYADTDTIVFGTGTKLTVL
V_{H}:
(SEQ ID NO: 25)
QVQLVQSEPEVKKPGSSVKLSCKASGGTFSTDAITWVRQAPGQGLEYMGGIIPLL
DSVDYAQRFQGRVTVSADKSTGTAYMEVRSLGSEDTAKYYCAKWSSVDTGLDYW
GQGTLVTVSS
P2AMR-12
V_{H}:
(SEQ ID NO: 26)
QVQLQESGPGLVKPSETLSLTCTVSGDSIINDPHYWGWIRQSPGKGLEWIGSTSHS
GHTYFNPSLKSRVSMSIDVAKNQFSLNVRSVTAADTAVYYCARMRYYYSGTYPVYY
FDYWGQGTLVTVSS
P2AMR-13
V_{L}:
(SEQ ID NO: 27)
SYVLTQPPSASGTPGQRVTISCSGSSSNIGSNFVSWYQQLPGTAPKLLISSNNQRPS
GVPDRFSGSKSDTSASLAISGLQSEDEADYYCAAWDDSLNGPVFGGGTQLTVL
V_{H}:
(SEQ ID NO: 28)
QVQLVQSEAEVKKPGSSVKVSCKASGGTFSRYGVSWVRQAPGQGLEWMGGIIP
MFGTANYAQKFQGRVTITADESTSTAYMELRSLRSEDTAVYYCARGDNSAYSDAFN
IWGQGTMVTVSS
AMRA3-2
V_{L}:
(SEQ ID NO: 32)
DIQMTQSPSSLSASVGDRVTITCRASQSVSSAVAWYQQKPGKAPKLLIYSASSLYS
GVPSRFSGSRSGTDFTLTISSLQPEDFATYYCQQSGWSYPITFGQGTKVEIKRTV
V_{H}:
(SEQ ID NO: 33)
EVQLVESGGGLVQPGGSLRLSCAASGFTFYSSYIHWVRQAPGKGLEWVASISPYY
GSTSYADSVKGRFTISADTSKNTAYLQMNSLRAEDTAVYYCARSSYYALDYWGQGT
LVTVSS
AMRA3-7
V_{L}:
(SEQ ID NO: 34)
DIQMTQSPSSLSASVGDRVTITCRASQSVSSAVAWYQQKPGKAPKLLIYSASSLYS
GVPSRFSGSRSGTDFTLTISSLQPEDFATYYCQQISYVYSLITFGQGTKVEIKRTV
V_{H}:
(SEQ ID NO: 35)
EVQLVESGGGLVQPGGSLRLSCAASGFTFSSYSIHWVRQAPGKGLEWVASIYSSY
GYTSYADSVKGRFTISADTSKNTAYLQMNSLRAEDTAVYYCARGGWYPAMDYWG
QGTLVTVSS
AMRA3-7KK
V_{L}:
(SEQ ID NO: 36)
DIQMTQSPSSLSASVGDRVTITCRASQSVSSAVAWYQQKPGKAPKLLIYSASSLYS
GVPSRFSGSRSGTDFTLTISSLQPEDFATYYCQQISYVKKLITFGQGTKVEIKRTV
V_{H}:
(SEQ ID NO: 37)
EVQLVESGGGLVQPGGSLRLSCAASGFTFSSYSIHWVRQAPGKGLEWVASIYSSY
GYTSYADSVKGRFTISADTSKNTAYLQMNSLRAEDTAVYYCARGGWYPAMDYWG
QGTLVTVSS
AMRA3-8
V_{L}:
(SEQ ID NO: 38)
DIQMTQSPSSLSASVGDRVTITCRASQSVSSAVAWYQQKPGKAPKLLIYSASSLYS
GVPSRFSGSRSGTDFTLTISSLQPEDLATYYCQQYQYGYNLITFGQGTKVEIKRTV
V_{H}:
(SEQ ID NO: 39)
EVQLVESGGGLVQPGGSLRLSCAASGFTISYSSIHWVRQAPGKGLEWVASIYSYS
GSTSYADSVKGRFTISADTSKNTAYLQMNSLRAEDTAVYYCARYSYGWVGPGWRAI
DYWGQGTLVTVSS
AMRA3-11
V_{L}:
(SEQ ID NO: 40)
DIQMTQSPSSLSASVGDRVTITCRASQSVSSAVAWYQQKPGKAPKLLIYSASSLYS
GVPSRFSGSRSGTDFTLTISSLQPEDFATYYCQQSSSVYKLLTFGQGTKVEIKRTV
V_{H}:
(SEQ ID NO: 41)
EVQLVESGGGLVQPGGSLRLSCAASGFTVYYSSIHWVRQAPGKGLEWVASISSSY
SYTSYADSVKGRFTISADTSKNTAYLQMNSLRAEDTALYYCARGGPGWYRAMDYW
GQGTLVTVSS
AMRA3-15
V_{L}:
(SEQ ID NO: 42)
DIQMTQSPSSLSASVGDRVTITCRASQSVSSAVAWYQQKPGKAPKLLIYSASSLYS
GVPSRFSGSRSGTDFTLTISSLQPEDFATYYCQQSSSSLITFGQGTKVEIKRTV
V_{H}:
(SEQ ID NO: 43)
EVQLVESGGGLVQPGGSLRLSCAASGFTFSSSSIHWVRQAPGKGLEWVASISSSSG
STSYADSVKGRFTISADTSKNTAYLQMNSLRAEDTAVYYCARGYFYYGWWAMAFD
YWGQGTLVTVSS
AMRA3-17
V_{L}:
(SEQ ID NO: 44)
DIQMTQSPSSLSASVGDRVTITCRASQSVSSAVAWYQQKPGKAPKLLIYSASSLYS
GVPSRFSGSRSGTDFTLTISSLQPEDFATYYCQQSQWYEPLITFGQGTKVEIKRTV
V_{H}:
(SEQ ID NO: 45)
EVQLVESGGGLVQPGGSLRLSCAASGFTIYSSYIHWVRQAPGKGLEWVASISSSS
GSTSYADSVKGRFTISADTSKNTAYLQMNSLRAEDTAVYYCARSYSYMSQWGWYQ
YSGMDYWGQGTLVTVSS
AMRA3-18
V_{L}:
(SEQ ID NO: 46)
DIQMTQSPSSLSASVGDRVTITCRASQSVSSAVAWYQQKPGKAPKLLIYSASSLYS
GVPSRFSGSRSGTDFTLTISSLQPEDFATYYCQQGSYTYRLITFGQGTKVEIKRTV
V_{H}:
(SEQ ID NO: 47)
EVQLVESGGGLVQPGGSLRLSCAASGFTVSYSSIHWVRQAPGKGLEWVASISSSS
GYTSYADSVKGRFTISADTSKNTAYLQMNSLRAEDTAVYYCARYAWWAHGLDYW
GQGTLVTVSS
AMRA3-21
V_{L}:
(SEQ ID NO: 48)
DIQMTQSPSSLSASVGDRVTITCRASQSVSSAVAWYQQKPGKAPKLLIYSASSLYS
GVPSRFSGSRSGTDFTLTISSLQPEDFATYYCQQASYWYNLFTFGQGTKVEIKRTV
V_{H}:
(SEQ ID NO: 49)
EVQLVESGGGLVQPGGSLRLSCAASGFTISSYSIHWVRQAPGKGLEWVASIYSSY
GYTSYADSVKGRFTISADTSKNTAYLQMNSLRAEDTAVYYCARQYSMHFPWGYGM
DYWGQGTLVTVSS
AMRA3-22
V_{L}:
(SEQ ID NO: 50)
DIQMTQSPSSLSASVGDRVTITCRASQSVSSAVAWYQQKPGKAPKLLIYSASSLYS
GVPSRFSGSRSGTDFTLTISSLQPEDFATYYCQQSDMPPITFGQGTKVEIKRTV
V_{H}:
(SEQ ID NO: 51)
EVQLVESGGGLVQPGGSLRLSCAASGFTFYSSSIHWVRQAPGKGLEWVAYIYSSS
GYTSYADSVKGRFTISADTSKNTAYLQMNSLRAEDTAVYYCARPVNYYYQGALDY
WGQGTLVTVSS
AMRA3-23
V_{L}:
(SEQ ID NO: 52)
DIQMTQSPSSLSASVGDRVTITCRASQSVSSAVAWYQQKPGKAPKLLIYSASSLYS
GVPSRFSGSRSGTDFTLTISSLQPEDFATYYCQQYYVFPITFGQGTKVEIKRTV
V_{H}:
(SEQ ID NO: 53)
EVQLVESGGGLVQPGGSLRLSCAASGFTVYSSSIHWVRQAPGKGLEWVASISPSS
GYTYYADSVKGRFTISADTSKNTAYLQMNSLRAEDTAVYYCARYHYMFEYDKGES
KWGYYGFDYWGQGTLVTVSS
AMRA11-1
V_{L}:
(SEQ ID NO: 54)
DIQMTQSPSSLSASVGDRVTITCRASQSVSSAVAWYQQKPGKAPKLLIYSASSLYS
GVPSRFSGSRSGTDFTLTISSLQPEDFATYYCQQSQYFPITFGQGTKVEIKRTV
V_{H}:
(SEQ ID NO: 55)
EVQLVESGGGLVQPGGSLRLSCAASGFTISYSSIHWVRQAPGKGLEWVASIYSYY
GYTSYADSVKGRFTISADTSKNTAYLQMNSLRAEDTAVYYCARNSWSWYSGVGMD
YWGQGTLVTVSS
AMRA11-2
V_{L}:
(SEQ ID NO: 56)
DIQMTQSPSSLSASVGDRVTITCRASQSVSSAVAWYQQKPGKAPKLLIYSASSLYS
GVPSRFSGSRSGTDFTLTISSLQPEDFATYYCQQSSSSLITFGQGTKVEIKRTV
V_{H}:
(SEQ ID NO: 57)
EVQLVESGGGLVQPGGSLRLSCAASGFTISSSSIHWVRQAPGKGLEWVASISSYSS
STYYADSVKGRFTISADTSKNTAYLQMNSLRAEDTAVYYCARYPYGWGWGGSGLD
YWGQGTLVTVSS
AMRA11-15
V_{L}:
(SEQ ID NO: 58)
DIQMTQSPSSLSASVGDRVTITCRASQSVSSAVAWYQQKPGKAPKLLIYSASSLYS
GVPSRFSGSRSGTDFTLTISSLQPEDFATYYCQQFDFQYLITFGQGTKVEIKRTV
V_{H}:
(SEQ ID NO: 59)
EVQLVESGGGLVQPGGSLRLSCAASGFTVYYSSIHWVRQAPGKGLEWVASIYSY
YGSTYYADSVKGRFTISADTSKNTAYLQMNSLRAEDTAVYYCARGEKWALDYWGQ
GTLVTVSS
AMRA11-16
V_{L}:
(SEQ ID NO: 60)
DIQMTQSPSSLSASVGDRVTITCRASQSVSSAVAWYQQKPGKAPKLLIYSASSLYS
GVPSRFSGSRSGTDFTLTISSLQPEDFATYYCQQYMYYQPLITFGQGTKVEIKRTV
V_{H}:
(SEQ ID NO: 61)
EVQLVESGGGLVQPGGSLRLSCAASGFTVYYSSIHWVRQAPGKGLEWVASISSSS
GSTSYADSVKGRFTISADTSKNTAYLQMNSLRAEDTAVYYCAREPYNYNWYGMDY
WGQGTLVTVSS
AMRA311-2
V_{L}:
(SEQ ID NO: 62)
DIQMTQSPSSLSASVGDRVTITCRASQSVSSAVAWYQQKPGKAPKLLIYSASSLYS
GVPSRFSGSRSGTDFTLTISSLQPEDFATYYCQQSLWWPITFGQGTKVEIKRTV
V_{H}:
(SEQ ID NO: 63)
EVQLVESGGGLVQPGGSLRLSCAASGFTVSSSSIHWVRQAPGKGLEWVASIYSYS
GYTSYADSVKGRFTISADTSKNTAYLQMNSLRAEDTAVYYCARHGSYGSWWALDY
WGQGTLVTVSS
AMRA311-10
V_{L}:
(SEQ ID NO: 64)
DIQMTQSPSSLSASVGDRVTITCRASQSVSSAVAWYQQKPGKAPKLLIYSASSLYS
GVPSRFSGSRSGTDFTLTISSLQPEDFATYYCQQYFYFPITFGQGTKVEIKRTV
V_{H}:
(SEQ ID NO: 65)
EVQLVESGGGLVQPGGSLRLSCAASGFTFYSSSIHWVRQAPGKGLEWVASISSYY
GSTSYADSVKGRFTISADTSKNTAYLQMNSLRAEDTAVYYCARASYYSGYGSSYPY
YMGLDYWGQGTLVTVSS
AMRA311-14
V_{L}:
(SEQ ID NO: 66)
DIQMTQSPSSLSASVGDRVTITCRASQSVSSAVAWYQQKPGKAPKLLIYSASSLYS
GVPSRFSGSRSGTDFTLTISSLQPEDFATYYCQQGSYRNPLLTFGQGTKVEIKRTV
V_{H}:
(SEQ ID NO: 67)
EVQLVESGGGLVQPGGSLRLSCAASGFTFSSYSIHWVRQAPGKGLEWVASISSSS
GYTSYADSVKGRFTISADTSKNTAYLQMNSLRAEDTAVYYCARMNWSHYAMDYW
GQGTLVTVSS
AMRA311-16
V_{L}:
(SEQ ID NO: 68)
DIQMTQSPSSLSASVGDRVTITCRASQSVSSAVAWYQQKPGKAPKLLIYSASSLYS
GVPSRFSGSRSGTDFTLTISSLQPEDFATYYCQQSSSSLITFGQGTKVEIKRTV
V_{H}:
(SEQ ID NO: 69)
EVQLVESGGGLVQPGGSLRLSCAASGFTISSSSIHWVRQAPGKGLEWVAYISSYS
GYTYYADSVKGRFTISADTSKNTAYLQMNSLRAEDTAVYYCARYWYGHYHSYFGL
DYWGQGTLVTVSS
AMRA311-17
V_{L}:
(SEQ ID NO: 70)
DIQMTQSPSSLSASVGDRVTITCRASQSVSSAVAWYQQKPGKAPKLLIYSASSLYS
GVPSRFSGSRSGTDFTLTISSLQPEDFATYYCQQSSSSLITFGQGTKVEIKRTV
V_{H}:
(SEQ ID NO: 71)
EVQLVESGGGLVQPGGSLRLSCAASGFTISSSSIHWVRQAPGKGLEWVASISSYSG
STYYADSVKGRFTISADTSKNTAYLQMNSLRAEDTAVYYCARYPYGSHVYTGLDY
WGQGTLVTVSS
AMRA311-18
V_{L}:
(SEQ ID NO: 72)
DIQMTQSPSSLSASVGDRVTITCRASQSVSSAVAWYQQKPGKAPKLLIYSASSLYS
GVPSRFSGSRSGTDFTLTISSLQPEDFATYYCQQWNWADYLVTFGQGTKVEIKRTV
V_{H}:
(SEQ ID NO: 73)
EVQLVESGGGLVQPGGSLRLSCAASGFTISSSSIHWVRQAPGKGLEWVASIYSSSG
STSYADSVKGRFTISADTSKNTAYLQMNSLRAEDTAVYYCARVYSSRYWGWGVAF
DYWGQGTLVTVSS
AMRA311-19
V_{L}:
(SEQ ID NO: 74)
DIQMTQSPSSLSASVGDRVTITCRASQSVSSAVAWYQQKPGKAPKLLIYSASSLYS
GVPSRFSGSRSGTDFTLTISSLQPEDFATYYCQQYYWYSLITFGQGTKVEIKRTV
V_{H}:
(SEQ ID NO: 75)
EVQLVESGGGLVQPGGSLRLSCAASGFTVYSSSIHWVRQAPGKGLEWVAYIYSSS
GYTSYADSVKGRFTISADTSKNTAYLQMNSLRAEDTAVYYCARRSFPQWYNGSYTP
WPAMDYWGQGTLVTVSS
AMRA311-20
V_{L}:
(SEQ ID NO: 76)
DIQMTQSPSSLSASVGDRVTITCRASQSVSSAVAWYQQKPGKAPKLLIYSASSLYS
GVPSRFSGSRSGTDFTLTISSLQPEDFATYYCQQYMWWPVTFGQGTKVEIKRTV
V_{H}:
(SEQ ID NO: 77)
EVQLVESGGGLVQPGGSLRLSCAASGFTVSSSSIHWVRQAPGKGLEWVASIYSYS
SYTSYADSVKGRFTISADTSKNTAYLQMNSLRAEDTAVYYCARPFYWGERYALDY
WGQGTLVTVSS
AMRA3-5
V_{L}:
(SEQ ID NO: 78)
DIQMTQSPSSLSASVGDRVTITCRASQSVSSAVAWYQQKPGKAPKLLIYSASSLYS
GVPSRFSGSRSGTDFTLTISSLQPEDFATYYCQQSYSTLVTFGQGTKVEIKRTV
V_{H}:
(SEQ ID NO: 79)
EVQLVESGGGLVQPGGSLRLSCAASGFTFYSSSIHWVRQAPGKGLEWVASIYSSY
GSTSYADSVKGRFTISADTSKNTAYLQMNSLRAEDTAVYYCARIYGWSYQGWAGM
DYWGQGTLVTVSS
AMRA3-6
V_{L}:
(SEQ ID NO: 80)
DIQMTQSPSSLSASVGDRVTITCRASQSVSSAVAWYQQKPGKAPKLLIYSASSLYS
GVPSRFSGSRSGTDFTLTISSLQPEDFATYYCQQSSSSLITFGQGTKVEIKRTV
V_{H}:
(SEQ ID NO: 81)
EVQLVESGGGLVQPGGSLRLSCAASGFTISYSSIHWVRQAPGKGLEWVASIYPYY
GSTSYADSVKGRFTISADTSKNTAYLQMNSLRAEDTAVYYCARGGDYYWGWYWV
AMDYWGQGTLVTVSS
AMRA3-9
V_{L}:
(SEQ ID NO: 82)
DIQMTQSPSSLSASVGDRVTITCRASQSVSSAVAWYQQKPGKAPKLLIYSASSLYS
GVPSRFSGSRSGTDFTLTIXSLQPEDFATYYCQKSSSSLITFGQGTKVEIKRTV
V_{H}:
(SEQ ID NO: 83)
EVQLVESGGGLVQPGGSLRLSCAASGFTFSSSYIHWVRQAPGKGLEWVASISSSY
GSTSYADSVKGRFTISADTSKNTAYLQMNSLRAEDTAVYYCARMYYYTYPGMDYW
GQGTLVTVSS
AMRA3-10
V_{L}:
(SEQ ID NO: 84)
DIQMTQSPSSLSASVGDRVTITCRASQSVSSAVAWYQQKPGKAPKLLIYSASSLYS
GVPSRFSGSRSGTDFTLTISSLQPEDFATYYCQKGSSYLLTFGQGTKVEIKRTV
V_{H}:
(SEQ ID NO: 85)
EVQLVESGGGLVQPGGSLRLSCAASGFTIYSYSIHWVRQAPGKGLEWVASISPSS
GYTSYADSVKGRFTISADTSKNTAYLQMNSLRAEDTAVYYCARYHYGGWSHYMSG
MDYWGQGTLVTVSS
AMRA3-13
V_{L}:
(SEQ ID NO: 86)
DIQMTQSPSSLSASVGDRVTITCRASQSVSSAVAWYQQKPGKAPKLLIYSASSLYS
GVPSRFSGSRSGTDFTLTISSLQPEDFATYYCQQNYYYHKLITFGQGTKVEIKRTV
V_{H}:
(SEQ ID NO: 87)
EVQLVESGGGLVQPGGSLRLSCAASGFTFSYSSIHWVRQAPGKGLEWVASISSSY
GYTSYADSVKGRFTISADTSKNTAYLQMNSLRAEDTAVYYCARGRYGGMDYWGQ
GTLVTVSS
AMRA3-19
V_{L}:
(SEQ ID NO: 88)
DIQMTQSPSSLSASVGDRVTITCRASQSVSSAVAWYQQKPGKAPKLLIYSASSLYS
GVPSRFSGSRSGTDFTLTISSLQPEDFATYYCQQLSYVYKLITFGQGTKVEIKRTV
V_{H}:
(SEQ ID NO: 89)
EVQLVESGGGLVQPGGSLRLSCAASGFTFYSSSIHWVRQAPGKGLEWVASISSSY
GYTSYADSVKGRFTISADTSKNTAYLQMNSLRAEDTAVYYCARGWYKAMDYWGQ
GTLVTVSS
AMRA3-24
V_{L}:
(SEQ ID NO: 90)
DIQMTQSPSSLSASVGDRVTITCRASQSVSSAVAWYQQKPGKAPKLLIYSASSLYS
GVPSRFSGSRSGTDFTLTISSLQPEDFATYYCQQSSSSLITFGQGTKVEIKRTV
V_{H}:
(SEQ ID NO: 91)
EVQLVESGGGLVQPGGSLRLSCAASGFTFSYSSIHWVRQAPGKGLEWVASISSSY
GSTYYADSVKGRFTISADTSKNTAYLQMNSLRAEDTAVYYCARMYYYYYPGIDYW
GQGTLVTVSS
AMRA11-3
V_{L}:
(SEQ ID NO: 92)
DIQMTQSPSSLSASVGDRVTITCRASQSVSSAVAWYQQKPGKAPKLLIYSASSLYS
GVPSRFSGSRSGTDFTLTISSLQPEDLATYYCQQYYYFPITFGQGTKVEIKRTV
V_{H}:
(SEQ ID NO: 93)
EVQLVESGGGLVQPGGSLRLSCAASGFTFSSSSIHWVRQAPGKGLEWVASISPYY
GYTSYADSVKGRFTISADTSKNTAYLQMNSLRAEDTAVYYCARSPYYWYQYFYGW
GLDYWGQGTLVTVSS
AMRA11-4
V_{L}:
(SEQ ID NO: 94)
DIQMTQSPSSLSASVGDRVTITCRASQSVSSAVAWYQQKPGKAPKLLIYSASSLYS
GVPSRFSGSRSGTDFTLTISSLQPEDFATYYCQQSSSSLITFGQGTKVEIKRTV
V_{H}:
(SEQ ID NO: 95)
EVQLVESGGGLVQPGGSLRLSCAASGFTFSYSSIHWVRQAPGKGLEWVASISSSS
GSTSYADSVKGRFTISADTSKNTAYLQMNSLRAEDTAVYYCARSPYWWNYMSAMD
YWGQGTLVTVSS
AMRA11-7
V_{L}:
(SEQ ID NO: 96)
DIQMTQSPSSLSASVGDRVTITCRASQSVSSAVAWYQQKPGKAPKLLIYSASSLYS
GVPSRFSGSRSGTDFTLTISSLQPEDFATYYCQQGWWWPFTFGQGTKVEIKRTV
V_{H}:
(SEQ ID NO: 97)
EVQLVESGGGLVQPGGSLRLSCAASGFTVSSYSIHWVRQAPGKGLEWVASISPYY
GYTSYADSVKGRFTISADTSKNTAYLQMNSLRAEDTAVYYCARWSWQYYSGHSSW
GLDYWGQGTLVTVSS
AMRA11-8
V_{L}:
(SEQ ID NO: 98)
DIQMTQSPSSLSASVGDRVTITCRASQSVSSAVAWYQQKPGKAPKLLIYSASSLYS
GVPSRFSGSRSGTDFTLTISSLQPEDFATYYCQQSWYFPLTFGQGTKVEIKRTV
V_{H}:
(SEQ ID NO: 99)
EVQLVESGGGLVQPGGSLRLSCAASGFTVSSSSIHWVRQAPGKGLEWVASIYSYY
GYTSYADSVKGRFTISADTSKNTAYLQMNSLRAEDTAVYYCARWYNEYYHDYYW
DAMDYWGQGTLVTVSS
AMRA11-9
V_{L}:
(SEQ ID NO: 100)
DIQMTQSPSSLSASVGDRVTITCRASQSVSSAVAWYQQKPGKAPKLLIYSASSLYS
GVPSRFSGSRSGTDFTLTISSLQPEDFATYYCQQSSSSLITFGQGTKVEIKRTV
V_{H}:
(SEQ ID NO: 101)
EVQLVESGGGLVQPGGSLRLSCAASGFTLYYSSIHWVRQAPGKGLEWVASISSSS
GSTSYADSVKGRFTISADTSKNTAYLQMNSLRAEDTAVYYCARWMYWWSFALDY
WGQGTLVTVSS
AMRA11-10
V_{L}:
(SEQ ID NO: 102)
DIQMTQSPSSLSASVGDRVTITCRASQSVSSAVAWYQQKPGKAPKLLIYSASSLYS
GVPSRFSGSRSGTDFTLTISSLQPEDFATYYCQQSYLWPITFGQGTKVEIKRTV
V_{H}:
(SEQ ID NO: 103)
EVQLVESGGGLVQPGGSLRLSCAASGFTVSSSSIHWVRQAPGKGLEWVASIYSYY
GYTSYADSVKGRFTISADTSKNTAYLQMNSLRAEDTAVYYCARWQYHYNYWYGM
DYWGQGTLVTVSS
AMRA11-11
V_{L}:
(SEQ ID NO: 104)
DIQMTQSPSSLSASVGDRVTITCRASQSVSSAVAWYQQKPGKAPKLLIYSASSLYS
GVPSRFSGSRSGTDFTLTISSLQPEDFATYYCQQYPMSLITFGQGTKVEIKRTV
V_{H}:
(SEQ ID NO: 105)
EVQLVESGGGLVQPGGSLRLSCAASGFTVSYSSIHWVRQAPGKGLEWVASISPYS
GYTSYADSVKGRFTISADTSKNTAYLQMNSLRAEDTAVYYCARGYDYYAGLDYWG
QGTLVTVSS
AMRA11-12
V_{L}:
(SEQ ID NO: 106)
DIQMTQSPSSLSASVGDRVTITCRASQSVSSAVAWYQQKPGKAPKLLIYSASSLYS
GVPSRFSGSRSGTDFTLTISSLQPEDFATYYCQQYYYFPITFGQGTKVEIKRTV
V_{H}:
(SEQ ID NO: 107)
EVQLVESGGGLVQPGGSLRLSCAASGFTFSYYSIHWVRQAPGKGLEWVASISPYY
GYTSYADSVKGRFTISADTSKNTAYLQMNSLRAEDTAVYYCARWESEYSGTYEDY
WAGMDYWGQGTLVTVSS
AMRA11-13
V_{L}:
(SEQ ID NO: 108)
DIQMTQSPSSLSASVGDRVTITCRASQSVSSAVAWYQQKPGKAPKLLIYSASSLYS
GVPSRFSGSRSGTDFTLTISSLQPEDFATYYCQQYMWWPITFGQGTKVEIKRTV
V_{H}:
(SEQ ID NO: 109)
EVQLVESGGGLVQPGGSLRLSCAASGFTISYSSIHWVRQAPGKGLEWVASISSSYS
YTSYADSVKGRFTISADTSKNTAYLQMNSLRAEDTAVYYCARTGYWQGYLALDYW
GQGTLVTVSS
AMRA11-14
V_{L}:
(SEQ ID NO: 110)
DIQMTQSPSSLSASVGDRVTITCRASQSVSSAVAWYQQKPGKAPKLLIYSASSLYS
GVPSRFSGSRSGTDFTLTISSLQPEDFATYYCQQSSSSLITFGQGTKVEIKRTV
V_{H}:
(SEQ ID NO: 111)
EVQLVESGGGLVQPGGSLRLSCAASGFTISYSSIHWVRQAPGKGLEWVASISSSSG
STSYADSVKGRFTISADTSKNTAYLQMNSLRAEDTAVYYCARTYYYYWNSTPAMD
YWGQGTLVTVSS
AMRA11-18
V_{L}:
(SEQ ID NO: 112)
DIQMTQSPSSLSASVGDRVTITCRASQSVSSAVAWYQQKPGKAPKLLIYSASSLYS
GVPSRFSGSRSGTDFTLTISSLQPEDFATYYCQQSYGYPVTFGQGTKVEIKRTV
V_{H}:
(SEQ ID NO: 113)
EVQLVESGGGLVQPGGSLRLSCAASGFTFSSSSIHWVRQAPGKGLEWVASISSSY
GYTSYADSVKGRFTISADTSKNTAYLQMNSLRAEDTAVYYCARWYNSSWYYSNW
WYKGFGMDYWGQGTLVTVSS
AMRA11-20
V_{L}:
(SEQ ID NO: 114)
DIQMTQSPSSLSASVGDRVTITCRASQSVSSAVAWYQQKPGKAPKLLIYSASSLYS
GVPSRFSGSRSGTDFTLTISSLQPEDFATYYCQQYYSSLFTFGQGTKVEIKRTV
V_{H}:
(SEQ ID NO: 115)
EVQLVESGGGLVQPGGSLRLSCAASGFTFYSSSIHWVRQAPGKGLEWVASISSSY
GYTSYADSVKGRFTISADTSKNTAYLQMNSLRAEDTAVYYCARTSYTYPVYTYYGF
DYWGQGTLVTVSS
AMRA11-22
V_{L}:
(SEQ ID NO: 116)
DIQMTQSPSSLSASVGDRVTITCRASQSVSSAVAWYQQKPGKAPKLLIYSASSLYS
GVPSRFSGSRSGTDFTLTISSLQPEDFATYYCQQSWYYPLTFGQGTKVEIKRTV
V_{H}:
(SEQ ID NO: 117)
EVQLVESGGGLVQPGGSLRLSCAASGFTLYSSSIHWVRQAPGKGLEWVASISSSY
GYTSYADSVKGRFTISADTSKNTAYLQMNSLRAEDTAVYYCARYRYSSWNRGAIDY
WGQGTLVTVSS
AMRA11-24
V_{L}:
(SEQ ID NO: 118)
DIQMTQSPSSLSASVGDRVTITCRASQSVSSAVAWYQQKPGKAPKLLIYSASSLYS
GVPSRFSGSRSGTDFTLTISSLQPEDFATYYCQQSYWWPLTFGQGTKVEIKRTV
V_{H}:
(SEQ ID NO: 119)
EVQLVESGGGLVQPGGSLRLSCAASGFTVSSSSIHWVRQAPGKGLEWVASIYSYY
GYTSYADSVKGRFTISADTSKNTAYLQMNSLRAEDTAVYYCARWSKSPWYYQGID
YWGQGTLVTVSS
IBA1-4
V_{L}:
(SEQ ID NO: 120)
DIQMTQSPSSLSASVGDRVTITCRASQSVSSAVAWYQQKPGKAPKLLIYSASSLYS
GVPSRFSGSRSGTDFTLTISSLQPEDFATYYCQQYHYWASLITFGQGTKVEIKRTV
V_{H}:
(SEQ ID NO: 121)
EVQLVESGGGLVQPGGSLRLSCAASGFTVSSSSIHWVRQAPGKGLEWVASIYSYS
GSTYYADSVKGRFTISADTSKNTAYLQMNSLRAEDTAVYYCARQYSSSYYVWPGM
DYWGQGTLVTVSS
IBA1-7
V_{L}:
(SEQ ID NO: 122)
DIQMTQSPSSLSASVGDRVTITCRASQSVSSAVAWYQQKPGKAPKLLIYSASSLYS
GVPSRFSGSRSGTDFTLTISSLQPEDFATYYCQQSYWWKSLVTFGQGTKVEIKRTV
V_{H}:
(SEQ ID NO: 123)
EVQLVESGGGLVQPGGSLRLSCAASGFTLSSSSIHWVRQAPGKGLEWVASISSYY
GSTSYADSVKGRFTISADTSKNTAYLQMNSLRAEDTAVYYCARMHYSWQEYYSYD
WGMDYWGQGTLVTVSS
IBA1-8
V_{L}:
(SEQ ID NO: 124)
DIQMTQSPSSLSASVGDRVTITCRASQSVSSAVAWYQQKPGKAPKLLIYSASSLYS
GVPSRFSGSRSGTDFTLTISSLQPEDFATYYCQQPYYPLITFGQGTKVEIKRTV
V_{H}:
(SEQ ID NO: 125)
EVQLVESGGGLVQPGGSLRLSCAASGFTISYSSIHWVRQAPGKGLEWVASIYPSY
GSTSYADSVKGRFTISADTSKNTAYLQMNSLRAEDTAVYYCARWQGYYQPALDYW
GQGTLVTVSS
IBA1-12
V_{L}:
(SEQ ID NO: 126)
DIQMTQSPSSLSASVGDRVTITCRASQSVSSAVAWYQQKPGKAPKLLIYSASSLYS
GVPSRFSGSRSGTDFTLTISSLQPEDFATYYCQQSSKYYYPITFGQGTKVEIKRTV
V_{H}:
(SEQ ID NO: 127)
EVQLVESGGGLVQPGGSLRLSCAASGFTISSSSIHWVRQAPGKGLEWVASISPYY
GYTSYADSVKGRFTISADTSKNTAYLQMNSLRAEDTAVYYCARWGYGWYWYGLD
YWGQGTLVTVSS
IBA1-13
V_{L}:
(SEQ ID NO: 128)
DIQMTQSPSSLSASVGDRVTITCRASQSVSSAVAWYQQKPGKAPKLLIYSASSLYS
GVPSRFSGSRSGTDFTLTISSLQPEDFATYYCQQGHDMNPVTFGQGTKVEIKRTV
V_{H}:
(SEQ ID NO: 129)
EVQLVESGGGLVQPGGSLRLSCAASGFTLSSSSIHWVRQAPGKGLEWVASIYSSY
GYTYYADSVKGRFTISADTSKNTAYLQMNSLRAEDTAVYYCARYYYYWYGGMDY
WGQGTLVTVSS
IBA1-19
V_{L}:
(SEQ ID NO: 130)
DIQMTQSPSSLSASVGDRVTITCRASQSVSSAVAWYQQKPGKAPKLLIYSASSLYS
GVPSRFSGSRSGTDFTLTISSLQPEDFATYYCQQSWMSDSLITFGQGTKVEIKRTV
V_{H}:
(SEQ ID NO: 131)
EVQLVESGGGLVQPGGSLRLSCAASGFTFSYSSIHWVRQAPGKGLEWVASIYPSS
GYTSYADSVKGRFTISADTSKNTAYLQMNSLRAEDTAVYYCARGWWYWMAWDY
AMDYWGQGTLVTVSS
IBA1-21
V_{L}:
(SEQ ID NO: 132)
DIQMTQSPSSLSASVGDRVTITCRASQSVSSAVAWYQQKPGKAPKLLIYSASSLYS
GVPSRFSGSRSGTDFTLTISSLQPEDFATYYCQQMQYSGWLITFGQGTKVEIKRTV
V_{H}:
(SEQ ID NO: 133)
EVQLVESGGGLVQPGGSLRLSCAASGFTISSSSIHWVRQAPGKGLEWVASISSYY
GYTSYADSVKGRFTISADTSKNTAYLQMNSLRAEDTAVYYCARYYSYSSGYGYYDY
FDWGAMDYWGQGTLVTVSS
OEA2-1
V_{L}:
(SEQ ID NO: 134)
DIQMTQSPSSLSASVGDRVTITCRASQSVSSAVAWYQQKPGKAPKLLIYSASSLYS
GVPSRFSGSRSGTDFTLTISSLQPEDFATYYCQQSSSSLITFGQGTKVEIKRTV
V_{H}:
(SEQ ID NO: 135)
EVQLVESGGGLVQPGGSLRLSCAASGFTFSSSSIHWVRQAPGKGLEWVASISSSSG
STSYADSVKGRFTISADTSKNTAYLQMNSLRAEDTAVYYCARYYGYVWGGYWGW
WYSKALDYWGQGTLVTVSS
OEA2-12
V_{L}:
(SEQ ID NO: 136)
DIQMTQSPSSLSASVGDRVTITCRASQSVSSAVAWYQQKPGKAPKLLIYSASSLYS
GVPSRFSGSRSGTDFTLTISSLQPEDFATYYCQQYDWNYYLVTFGQGTKVEIKRTV
V_{H}:
(SEQ ID NO: 137)
EVQLVESGGGLVQPGGSLRLSCAASGFTIYSSSIHWVRQAPGKGLEWVASISSYY
GYTSYADSVKGRFTISADTSKNTAYLQMNSLRAEDTAVYYCARYQYYGSLYYSQQ
WAMDYWGQGTLVTVSS
OEA2-16
V_{L}:
(SEQ ID NO: 138)
DIQMTQSPSSLSASVGDRVTITCRASQSVSSAVAWYQQKPGKAPKLLIYSASSLYS
GVPSRFSGSRSGTDFTLTISSLQPEDFATYYCQQSSSSLITFGQGTKVEIKRTV
V_{H}:
(SEQ ID NO: 139)
EVQLVESGGGLVQPGGSLRLSCAASGFTFSSSSIHWVRQAPGKGLEWVASISSSSG
STSYADSVKGRFTISADTSKNTAYLQMNSLRAEDTAVYYCARSPSSPYFMSWGWYW
QYGIDYWGQGTLVTVSS
OEA2-21
V_{L}:
(SEQ ID NO: 140)
DIQMTQSPSSLSASVGDRVTITCRASQSVSSAVAWYQQKPGKAPKLLIYSASSLYS
GVPSRFSGSRSGTDFTLTISSLQPEDFATYYCQQSSWGGLVTFGQGTKVEIKRTV
V_{H}:
(SEQ ID NO: 141)
EVQLVESGGGLVQPGGSLRLSCAASGFTFSSSYIHWVRQAPGKGLEWVASISPSY
GYTSYADSVKGRFTISADTSKNTAYLQMNSLRAEDTAVYYCARDMYEWWHWAID
YWGQGTLVTVSS
OEA2-24
V_{L}:
(SEQ ID NO: 142)
DIQMTQSPSSLSASVGDRVTITCRASQSVSSAVAWYQQKPGKAPKLLIYSASSLYS
GVPSRFSGSRSGTDFTLTISSLQPEDFATYYCQQSSSSLITFGQGTKVEIKRTV
V_{H}:
(SEQ ID NO: 143)
EVQLVESGGGLVQPGGSLRLSCAASGFTFSSSSIHWVRQAPGKGLEWVASISSSSG
STSYADSVKGRFTISADTSKNTAYLQMNSLRAEDTAVYYCARYGHYLYYWGWGW
YWSAALDYWGQGTLVTVSS

24. The binding partner of claim 9, wherein the binding partner comprises a light chain that comprises a complementary determining region 3 (CDR3) that comprises the sequence SEQ ID NO:154 and a heavy chain that comprises a CDR3 that comprises the sequence of SEQ ID NO:156, or wherein the binding partner comprises a light chain that comprises a CDR3 that comprises the sequence of SEQ ID NO:158 and a heavy chain that comprises a CDR3 that comprises the sequence of SEQ ID NO: 160.

25. The binding partner of claim 9, wherein the binding partner is a single-chain Diabody (scDb) and comprises a sequence that is at least 95% identical to SEQ ID NO: 165, SEQ ID NO: 161, SEQ ID NO:162, SEQ ID NO: 163, or SEQ ID NO: 164.

26. The binding partner of claim 9, wherein the binding partner is in a CrossMab format and comprises the sequence of SEQ ID NO:149.

27. The binding partner of claim 24, wherein the binding partner is comprised by a chimeric antigen receptor.

28. The binding partner of claim 27, wherein the binding partner is expressed by a T cell or a natural killer cell.

29. A complex comprising a binding agent of claim 9 and the peptide conjugate.

30. A binding partner of claim 9, wherein the binding partner comprises an Fc segment.

31. A polynucleotide encoding a binding partner of claim 9, wherein the polynucleotide comprises DNA or mRNA, and wherein if the polynucleotide comprises DNA, the DNA is optionally a component of an expression vector.

32. A eukaryotic cell comprising a polynucleotide encoding a binding partner of claim 9, wherein the cell is optionally a totipotent, multipotent, or pluripotent stem cell, wherein optionally the stem cell has an induced stem cell phenotype, or wherein the cell is optionally a leukocyte, and wherein the leukocyte is optionally a T cell or a natural killer cell.

33. A method comprising administering to an individual in need thereof a composition comprising a binding partner of claim 9, or a polynucleotide encoding said binding partner.

34. The method of claim 33, wherein the binding partner is conjugated to a chemotherapeutic agent, a radioisotope, or a toxin.

35. A method comprising administering to an individual in need thereof a cell of claim 32.

36. A method comprising detecting a peptide conjugate, the method comprising forming a complex comprising the peptide conjugate and binding partner as in claim 9, wherein the binding partner is detectably labeled, and detecting the presence of the detectably labeled binding partner to determine the presence of the peptide conjugate.

37. The method of claim 36, wherein detecting the binding partner comprises a diagnostic indication, and wherein the binding partner optionally comprises a radioconjugate.

38. The method of claim 36, wherein the binding partner binds a peptide conjugate, and wherein said peptide conjugate is present on the surface of a cancer cell, wherein optionally the cancer cell is present in a tumor.

39. The method of claim 36, comprising screening a plurality of molecules for binding to a protein or peptide comprising a covalently attached molecule using a binding partner of claim 9.


SEQUENCE LISTING (165 sequences)

SEQ ID NO: 1 - protein, 9 aa, Artificial Sequence
  Description of Artificial Sequence Synthetic peptide
VVGACGVGK

SEQ ID NO: 2 - protein, 10 aa, Artificial Sequence
  Description of Artificial Sequence Synthetic peptide
VVVGACGVGK

SEQ ID NO: 3 - protein, 112 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQISYVKKLITFGQGTKVEIKRTV

SEQ ID NO: 4 - protein, 118 aa, Artificial Sequence
  Description of Artificial Sequence Synthetic polypeptide
EVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGGWYPAMDYWGQGTLVTVSS

SEQ ID NO: 5 - protein, 110 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYSYWPITFGQGTKVEIKRTV

SEQ ID NO: 6 - protein, 118 aa, Artificial Sequence
  Description of Artificial Sequence Synthetic polypeptide
EVQLVESGGGLVQPGGSLRLSCAASGFTISSSYIHWVRQAPGKGLEWVAYISPSYGSTSYADSVKGRFTISADTSKNTAYLQMNSLRAEDTAVYYCAREYVTMALDYWGQGTLVTVSS

SEQ ID NO: 7 - protein, 18 aa, Thosea asigna virus
EGRGSLLTCGDVEENPGP

SEQ ID NO: 8 - protein, 17 aa, Teschovirus A
ATNFSLKQAGDVENPGP

SEQ ID NO: 9 - protein, 19 aa, Equine rhinitis A virus
QCTNYALKLAGDVESNPGP

SEQ ID NO: 10 - protein, 21 aa, Foot-and-mouth disease virus
VKQTLNFDLKLAGDVESNPGP

SEQ ID NO: 11 - protein, 8 aa, Artificial Sequence
  Description of Artificial Sequence Synthetic peptide
GGGGSAAA

SEQ ID NO: 12 - protein, 20 aa, Artificial Sequence
  Description of Artificial Sequence Synthetic peptide
GGGGSGGGGSGGGGSGGGGS

SEQ ID NO: 13 - protein, 6 aa, Artificial Sequence
  Description of Artificial Sequence Synthetic 6xHis tag
HHHHHH

SEQ ID NO: 14 - protein, 107 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQDWYFPITFGQGTKVEIK

SEQ ID NO: 15 - protein, 128 aa, Artificial Sequence
  Description of Artificial Sequence Synthetic polypeptide
EVQLVESGGGLVQPGGSLRLSCAASGFTFSSYYIHWVRQAPGKGLEWVASISPSSGSTYYADSVKGRFTISADTSKNTAYLQMNSLRAEDTAVYYCARYGGRSYWQKQDSYFYQHGLDYWGQGTLVSS

SEQ ID NO: 16 - protein, 107 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIK

SEQ ID NO: 17 - protein, 125 aa, Artificial Sequence
  Description of Artificial Sequence Synthetic polypeptide
EVQLVESGGGLVQPGGSLRLSCAASGFTFSSSSIHWVRQAPGKGLEWVASISSYSGYTSYADSVKGRFTISADTSKNTAYLQMNSLRAEDTAVYYCARSYSYSEFRYYYSGQGMDYWGQGTLVSS

SEQ ID NO: 18 - protein, 107 aa, Artificial Sequence; identical to SEQ ID NO: 16
  Description of Artificial Sequence Synthetic polypeptide

SEQ ID NO: 19 - protein, 123 aa, Artificial Sequence
  Description of Artificial Sequence Synthetic polypeptide
EVQLVESGGGLVQPGGSLRLSCAASGFTFSSSSIHWVRQAPGKGLEWVASISSSSGSTSYADSVKGRFTISADTSKNTAYLQMNSLRAEDTAVYYCARSNYGWRWHLVGMDYWGQGTLVTVSS

SEQ ID NO: 20 - protein, 107 aa, Artificial Sequence; identical to SEQ ID NO: 16
  Description of Artificial Sequence Synthetic polypeptide

SEQ ID NO: 21 - protein, 123 aa, Artificial Sequence
  Description of Artificial Sequence Synthetic polypeptide
EVQLVESGGGLVQPGGSLRLSCAASGFTFSSSSIHWVRQAPGKGLEWVASISSSSGSTSYADSVKGRFTISADTSKNTAYLQMNSLRAEDTAVYYCARSPYVYYWYMVGFDYWGQGTLVTVSS

SEQ ID NO: 22 - protein, 15 aa, Artificial Sequence
  Description of Artificial Sequence Synthetic peptide
YKLVVVGACGVGKSA

SEQ ID NO: 23 - protein, 10 aa, Artificial Sequence
  Description of Artificial Sequence Synthetic peptide
SSSSCSSSSW

SEQ ID NO: 24 - protein, 110 aa, Artificial Sequence
  Description of Artificial Sequence Synthetic polypeptide
QSVLIQPRSVSGSPGQSVTISCTGTSSDVGGYNYVSWYQQHPGKAPKLMIYDVSKRPSGVPDRFSGSKSGNTASLTVSGLQAEDEADYYCGSYADTDTIVFGTGTKLTVL

SEQ ID NO: 25 - protein, 119 aa, Artificial Sequence
  Description of Artificial Sequence Synthetic polypeptide
QVQLVQSEPEVKKPGSSVKLSCKASGGTFSTDAITWVRQAPGQGLEYMGGIIPLLDSVDYAQRFQGRVTVSADKSTGTAYMEVRSLGSEDTAKYYCAKWSSVDTGLDYWGQGTLVTVSS

SEQ ID NO: 26 - protein, 126 aa, Artificial Sequence
  Description of Artificial Sequence Synthetic polypeptide
QVQLQESGPGLVKPSETLSLTCTVSGDSIINDPHYWGWIRQSPGKGLEWIGSTSHSGHTYFNPSLKSRVSMSIDVAKNQFSLNVRSVTAADTAVYYCARMRYYYSGTYPVYYFDYWGQGTLVTVSS

SEQ ID NO: 27 - protein, 110 aa, Artificial Sequence
  Description of Artificial Sequence Synthetic polypeptide
SYVLTQPPSASGTPGQRVTISCSGSSSNIGSNFVSWYQQLPGTAPKLLISSNNQRPSGVPDRFSGSKSDTSASLAISGLQSEDEADYYCAAWDDSLNGPVFGGGTQLTVL

SEQ ID NO: 28 - protein, 121 aa, Artificial Sequence
  Description of Artificial Sequence Synthetic polypeptide
QVQLVQSEAEVKKPGSSVKVSCKASGGTFSRYGVSWVRQAPGQGLEWMGGIIPMFGTANYAQKFQGRVTITADESTSTAYMELRSLRSEDTAVYYCARGDNSAYSDAFNIWGQGTMVTVSS

SEQ ID NO: 29 - protein, 9 aa, Homo sapiens
VVGAGGVGK

SEQ ID NO: 30 - protein, 9 aa, Artificial Sequence
  Description of Artificial Sequence Synthetic peptide
QLMPFGCLL

SEQ ID NO: 31 - protein, 10 aa, Artificial Sequence
  Description of Artificial Sequence Synthetic peptide
YMANGCLLNY

SEQ ID NO: 32 - protein, 111 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSGWSYPITFGQGTKVEIKRTV

SEQ ID NO: 33 - protein, 117 aa, Artificial Sequence
  Description of Artificial Sequence Synthetic polypeptide
EVQLVESGGGLVQPGGSLRLSCAASGFTFYSSYIHWVRQAPGKGLEWVASISPYYGSTSYADSVKGRFTISADTSKNTAYLQMNSLRAEDTAVYYCARSSYYALDYWGQGTLVTVSS

SEQ ID NO: 34 - protein, 112 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQISYVYSLITFGQGTKVEIKRTV

SEQ ID NO: 35 - protein, 118 aa, Artificial Sequence
  Description of Artificial Sequence Synthetic polypeptide
EVQLVESGGGLVQPGGSLRLSCAASGFTFSSYSIHWVRQAPGKGLEWVASIYSSYGYTSYADSVKGRFTISADTSKNTAYLQMNSLRAEDTAVYYCARGGWYPAMDYWGQGTLVTVSS

SEQ ID NO: 36 - protein, 112 aa, Artificial Sequence; identical to SEQ ID NO: 3
  Description of Artificial Sequence Synthetic polypeptide

SEQ ID NO: 37 - protein, 118 aa, Artificial Sequence; identical to SEQ ID NO: 35
  Description of Artificial Sequence Synthetic polypeptide

SEQ ID NO: 38 - protein, 112 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDLATYYCQQYQYGYNLITFGQGTKVEIKRTV

SEQ ID NO: 39 - protein, 124 aa, Artificial Sequence
  Description of Artificial Sequence Synthetic polypeptide
EVQLVESGGGLVQPGGSLRLSCAASGFTISYSSIHWVRQAPGKGLEWVASIYSYSGSTSYADSVKGRFTISADTSKNTAYLQMNSLRAEDTAVYYCARYSYGWVGPGWRAIDYWGQGTLVTVSS

SEQ ID NO: 40 - protein, 112 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSSSVYKLLTFGQGTKVEIKRTV

SEQ ID NO: 41 - protein, 120 aa, Artificial Sequence
  Description of Artificial Sequence Synthetic polypeptide
EVQLVESGGGLVQPGGSLRLSCAASGFTVYYSSIHWVRQAPGKGLEWVASISSSYSYTSYADSVKGRFTISADTSKNTAYLQMNSLRAEDTALYYCARGGPGWYRAMDYWGQGTLVTVSS

SEQ ID NO: 42 - protein, 110 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKRTV

SEQ ID NO: 43 - protein, 123 aa, Artificial Sequence
  Description of Artificial Sequence Synthetic polypeptide
EVQLVESGGGLVQPGGSLRLSCAASGFTFSSSSIHWVRQAPGKGLEWVASISSSSGSTSYADSVKGRFTISADTSKNTAYLQMNSLRAEDTAVYYCARGYFYYGWWAMAFDYWGQGTLVTVSS

SEQ ID NO: 44 - protein, 112 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSQWYEPLITFGQGTKVEIKRTV

SEQ ID NO: 45 - protein, 127 aa, Artificial Sequence
  Description of Artificial Sequence Synthetic polypeptide
EVQLVESGGGLVQPGGSLRLSCAASGFTIYSSYIHWVRQAPGKGLEWVASISSSSGSTSYADSVKGRFTISADTSKNTAYLQMNSLRAEDTAVYYCARSYSYMSQWGWYQYSGMDYWGQGTLVTVSS

SEQ ID NO: 46 - protein, 112 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQGSYTYRLITFGQGTKVEIKRTV

SEQ ID NO: 47 - protein, 119 aa, Artificial Sequence
  Description of Artificial Sequence Synthetic polypeptide
EVQLVESGGGLVQPGGSLRLSCAASGFTVSYSSIHWVRQAPGKGLEWVASISSSSGYTSYADSVKGRFTISADTSKNTAYLQMNSLRAEDTAVYYCARYAWWAHGLDYWGQGTLVTVSS

SEQ ID NO: 48 - protein, 112 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQASYWYNLFTFGQGTKVEIKRTV

SEQ ID NO: 49 - protein, 123 aa, Artificial Sequence
  Description of Artificial Sequence Synthetic polypeptide
EVQLVESGGGLVQPGGSLRLSCAASGFTISSYSIHWVRQAPGKGLEWVASIYSSYGYTSYADSVKGRFTISADTSKNTAYLQMNSLRAEDTAVYYCARQYSMHFPWGYGMDYWGQGTLVTVSS

SEQ ID NO: 50 - protein, 110 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSDMPPITFGQGTKVEIKRTV

SEQ ID NO: 51 - protein, 121 aa, Artificial Sequence
  Description of Artificial Sequence Synthetic polypeptide
EVQLVESGGGLVQPGGSLRLSCAASGFTFYSSSIHWVRQAPGKGLEWVAYIYSSSGYTSYADSVKGRFTISADTSKNTAYLQMNSLRAEDTAVYYCARPVNYYYQGALDYWGQGTLVTVSS

SEQ ID NO: 52 - protein, 110 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYYVFPITFGQGTKVEIKRTV

SEQ ID NO: 53 - protein, 130 aa, Artificial Sequence
  Description of Artificial Sequence Synthetic polypeptide
EVQLVESGGGLVQPGGSLRLSCAASGFTVYSSSIHWVRQAPGKGLEWVASISPSSGYTYYADSVKGRFTISADTSKNTAYLQMNSLRAEDTAVYYCARYHYMFEYDKGESKWGYYGFDYWGQGTLVTVSS

SEQ ID NO: 54 - protein, 110 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSQYFPITFGQGTKVEIKRTV

SEQ ID NO: 55 - protein, 122 aa, Artificial Sequence
  Description of Artificial Sequence Synthetic polypeptide
EVQLVESGGGLVQPGGSLRLSCAASGFTISYSSIHWVRQAPGKGLEWVASIYSYYGYTSYADSVKGRFTISADTSKNTAYLQMNSLRAEDTAVYYCARNSWSWYSGVGMDYWGQGTLVTVSS

SEQ ID NO: 56 - protein, 110 aa, Artificial Sequence; identical to SEQ ID NO: 42
  Description of Artificial Sequence Synthetic polypeptide

SEQ ID NO: 57 - protein, 123 aa, Artificial Sequence
  Description of Artificial Sequence Synthetic polypeptide
EVQLVESGGGLVQPGGSLRLSCAASGFTISSSSIHWVRQAPGKGLEWVASISSYSSSTYYADSVKGRFTISADTSKNTAYLQMNSLRAEDTAVYYCARYPYGWGWGGSGLDYWGQGTLVTVSS

SEQ ID NO: 58 - protein, 111 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQFDFQYLITFGQGTKVEIKRTV

SEQ ID NO: 59 - protein, 117 aa, Artificial Sequence
  Description of Artificial Sequence Synthetic polypeptide
EVQLVESGGGLVQPGGSLRLSCAASGFTVYYSSIHWVRQAPGKGLEWVASIYSYYGSTYYADSVKGRFTISADTSKNTAYLQMNSLRAEDTAVYYCARGEKWALDYWGQGTLVTVSS

SEQ ID NO: 60 - protein, 112 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYMYYQPLITFGQGTKVEIKRTV

SEQ ID NO: 61 - protein, 121 aa, Artificial Sequence
  Description of Artificial Sequence Synthetic polypeptide
EVQLVESGGGLVQPGGSLRLSCAASGFTVYYSSIHWVRQAPGKGLEWVASISSSSGSTSYADSVKGRFTISADTSKNTAYLQMNSLRAEDTAVYYCAREPYNYNWYGMDYWGQGTLVTVSS

SEQ ID NO: 62 - protein, 110 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSLWWPITFGQGTKVEIKRTV

SEQ ID NO: 63 - protein, 121 aa, Artificial Sequence
  Description of Artificial Sequence Synthetic polypeptide
EVQLVESGGGLVQPGGSLRLSCAASGFTVSSSSIHWVRQAPGKGLEWVASIYSYSGYTSYADSVKGRFTISADTSKNTAYLQMNSLRAEDTAVYYCARHGSYGSWWALDYWGQGTLVTVSS

SEQ ID NO: 64 - protein, 110 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYFYFPITFGQGTKVEIKRTV

SEQ ID NO: 65 - protein, 128 aa, Artificial Sequence
  Description of Artificial Sequence Synthetic polypeptide
EVQLVESGGGLVQPGGSLRLSCAASGFTFYSSSIHWVRQAPGKGLEWVASISSYYGSTSYADSVKGRFTISADTSKNTAYLQMNSLRAEDTAVYYCARASYYSGYGSSYPYYMGLDYWGQGTLVTVSS

SEQ ID NO: 66 - protein, 112 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQGSYRNPLLTFGQGTKVEIKRTV

SEQ ID NO: 67 - protein, 119 aa, Artificial Sequence
  Description of Artificial Sequence Synthetic polypeptide
EVQLVESGGGLVQPGGSLRLSCAASGFTFSSYSIHWVRQAPGKGLEWVASISSSSGYTSYADSVKGRFTISADTSKNTAYLQMNSLRAEDTAVYYCARMNWSHYAMDYWGQGTLVTVSS

SEQ ID NO: 68 - protein, 110 aa, Artificial Sequence; identical to SEQ ID NO: 42
  Description of Artificial Sequence Synthetic polypeptide

SEQ ID NO: 69 - protein, 123 aa, Artificial Sequence
  Description of Artificial Sequence Synthetic polypeptide
EVQLVESGGGLVQPGGSLRLSCAASGFTISSSSIHWVRQAPGKGLEWVAYISSYSGYTYYADSVKGRFTISADTSKNTAYLQMNSLRAEDTAVYYCARYWYGHYHSYFGLDYWGQGTLVTVSS

SEQ ID NO: 70 - protein, 110 aa, Artificial Sequence; identical to SEQ ID NO: 42
  Description of Artificial Sequence Synthetic polypeptide

SEQ ID NO: 71 - protein, 122 aa, Artificial Sequence
  Description of Artificial Sequence Synthetic polypeptide
EVQLVESGGGLVQPGGSLRLSCAASGFTISSSSIHWVRQAPGKGLEWVASISSYSGSTYYADSVKGRFTISADTSKNTAYLQMNSLRAEDTAVYYCARYPYGSHVYTGLDYWGQGTLVTVSS

SEQ ID NO: 72 - protein, 112 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQWNWADYLVTFGQGTKVEIKRTV

SEQ ID NO: 73 - protein, 124 aa, Artificial Sequence
  Description of Artificial Sequence Synthetic polypeptide
EVQLVESGGGLVQPGGSLRLSCAASGFTISSSSIHWVRQAPGKGLEWVASIYSSSGSTSYADSVKGRFTISADTSKNTAYLQMNSLRAEDTAVYYCARVYSSRYWGWGVAFDYWGQGTLVTVSS

SEQ ID NO: 74 - protein, 111 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYYWYSLITFGQGTKVEIKRTV

SEQ ID NO: 75 - protein, 128 aa, Artificial Sequence
  Description of Artificial Sequence Synthetic polypeptide
EVQLVESGGGLVQPGGSLRLSCAASGFTVYSSSIHWVRQAPGKGLEWVAYIYSSSGYTSYADSVKGRFTISADTSKNTAYLQMNSLRAEDTAVYYCARRSFPQWYNGSYTPWPAMDYWGQGTLVTVSS

SEQ ID NO: 76 - protein, 110 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYMWWPVTFGQGTKVEIKRTV

SEQ ID NO: 77 - protein, 121 aa, Artificial Sequence
  Description of Artificial Sequence Synthetic polypeptide
EVQLVESGGGLVQPGGSLRLSCAASGFTVSSSSIHWVRQAPGKGLEWVASIYSYSSYTSYADSVKGRFTISADTSKNTAYLQMNSLRAEDTAVYYCARPFYWGERYALDYWGQGTLVTVSS

SEQ ID NO: 78 - protein, 110 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSYSTLVTFGQGTKVEIKRTV

SEQ ID NO: 79 - protein, 123 aa, Artificial Sequence
  Description of Artificial Sequence Synthetic polypeptide
EVQLVESGGGLVQPGGSLRLSCAASGFTFYSSSIHWVRQAPGKGLEWVASIYSSYGSTSYADSVKGRFTISADTSKNTAYLQMNSLRAEDTAVYYCARIYGWSYQGWAGMDYWGQGTLVTVSS

SEQ ID NO: 80 - protein, 110 aa, Artificial Sequence; identical to SEQ ID NO: 42
  Description of Artificial Sequence Synthetic polypeptide

SEQ ID NO: 81 - protein, 124 aa, Artificial Sequence
  Description of Artificial Sequence Synthetic polypeptide
EVQLVESGGGLVQPGGSLRLSCAASGFTISYSSIHWVRQAPGKGLEWVASIYPYYGSTSYADSVKGRFTISADTSKNTAYLQMNSLRAEDTAVYYCARGGDYYWGWYWVAMDYWGQGTLVTVSS

SEQ ID NO: 82 - protein, 110 aa, Artificial Sequence
  Description of Artificial Sequence Synthetic polypeptide
  MOD_RES (76)..(76): Any amino acid
DIQMTQSPSSLSASVGDRVTITCRASQSVSSAVAWYQQKPGKAPKLLIYSASSLYSGVPSRFSGSRSGTDFTLTIXSLQPEDFATYYCQKSSSSLITFGQGTKVEIKRTV

SEQ ID NO: 83 - protein, 120 aa, Artificial Sequence
  Description of Artificial Sequence Synthetic polypeptide
EVQLVESGGGLVQPGGSLRLSCAASGFTFSSSYIHWVRQAPGKGLEWVASISSSYGSTSYADSVKGRFTISADTSKNTAYLQMNSLRAEDTAVYYCARMYYYTYPGMDYWGQGTLVTVSS

SEQ ID NO: 84 - protein, 110 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQKGSSYLLTFGQGTKVEIKRTV

SEQ ID NO: 85 - protein, 124 aa, Artificial Sequence
  Description of Artificial Sequence Synthetic polypeptide
EVQLVESGGGLVQPGGSLRLSCAASGFTIYSYSIHWVRQAPGKGLEWVASISPSSGYTSYADSVKGRFTISADTSKNTAYLQMNSLRAEDTAVYYCARYHYGGWSHYMSGMDYWGQGTLVTVSS

SEQ ID NO: 86 - protein, 112 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQNYYYHKLITFGQGTKVEIKRTV

SEQ ID NO: 87 - protein, 117 aa, Artificial Sequence
  Description of Artificial Sequence Synthetic polypeptide
EVQLVESGGGLVQPGGSLRLSCAASGFTFSYSSIHWVRQAPGKGLEWVASISSSYGYTSYADSVKGRFTISADTSKNTAYLQMNSLRAEDTAVYYCARGRYGGMDYWGQGTLVTVSS

SEQ ID NO: 88 - protein, 112 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQLSYVYKLITFGQGTKVEIKRTV

SEQ ID NO: 89 - protein, 117 aa, Artificial Sequence
  Description of Artificial Sequence Synthetic polypeptide
EVQLVESGGGLVQPGGSLRLSCAASGFTFYSSSIHWVRQAPGKGLEWVASISSSYGYTSYADSVKGRFTISADTSKNTAYLQMNSLRAEDTAVYYCARGWYKAMDYWGQGTLVTVSS

SEQ ID NO: 90 - protein, 110 aa, Artificial Sequence; identical to SEQ ID NO: 42
  Description of Artificial Sequence Synthetic polypeptide

SEQ ID NO: 91 - protein, 120 aa, Artificial Sequence
  Description of Artificial Sequence Synthetic polypeptide
EVQLVESGGGLVQPGGSLRLSCAASGFTFSYSSIHWVRQAPGKGLEWVASISSSYGSTYYADSVKGRFTISADTSKNTAYLQMNSLRAEDTAVYYCARMYYYYYPGIDYWGQGTLVTVSS

SEQ ID NO: 92 - protein, 110 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDLATYYCQQYYYFPITFGQGTKVEIKRTV

SEQ ID NO: 93 - protein, 125 aa, Artificial Sequence
  Description of Artificial Sequence Synthetic polypeptide
EVQLVESGGGLVQPGGSLRLSCAASGFTFSSSSIHWVRQAPGKGLEWVASISPYYGYTSYADSVKGRFTISADTSKNTAYLQMNSLRAEDTAVYYCARSPYYWYQYFYGWGLDYWGQGTLVTVSS

SEQ ID NO: 94 - protein, 110 aa, Artificial Sequence; identical to SEQ ID NO: 42
  Description of Artificial Sequence Synthetic polypeptide

SEQ ID NO: 95 - protein, 122 aa, Artificial Sequence
  Description of Artificial Sequence Synthetic polypeptide
EVQLVESGGGLVQPGGSLRLSCAASGFTFSYSSIHWVRQAPGKGLEWVASISSSSGSTSYADSVKGRFTISADTSKNTAYLQMNSLRAEDTAVYYCARSPYWWNYMSAMDYWGQGTLVTVSS

SEQ ID NO: 96 - protein, 110 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQGWWWPFTFGQGTKVEIKRTV

SEQ ID NO: 97 - protein, 125 aa, Artificial Sequence
  Description of Artificial Sequence Synthetic polypeptide
EVQLVESGGGLVQPGGSLRLSCAASGFTVSSYSIHWVRQAPGKGLEWVASISPYYGYTSYADSVKGRFTISADTSKNTAYLQMNSLRAEDTAVYYCARWSWQYYSGHSSWGLDYWGQGTLVTVSS

SEQ ID NO: 98 - protein, 110 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSWYFPLTFGQGTKVEIKRTV

SEQ ID NO: 99 - protein, 125 aa, Artificial Sequence
  Description of Artificial Sequence Synthetic polypeptide
EVQLVESGGGLVQPGGSLRLSCAASGFTVSSSSIHWVRQAPGKGLEWVASIYSYYGYTSYADSVKGRFTISADTSKNTAYLQMNSLRAEDTAVYYCARWYNEYYHDYYWDAMDYWGQGTLVTVSS

SEQ ID NO: 100 - protein, 110 aa, Artificial Sequence; identical to SEQ ID NO: 42
  Description of Artificial Sequence Synthetic polypeptide

SEQ ID NO: 101 - protein, 120 aa, Artificial Sequence
  Description of Artificial Sequence Synthetic polypeptide
EVQLVESGGGLVQPGGSLRLSCAASGFTLYYSSIHWVRQAPGKGLEWVASISSSSGSTSYADSVKGRFTISADTSKNTAYLQMNSLRAEDTAVYYCARWMYWWSFALDYWGQGTLVTVSS

SEQ ID NO: 102 - protein, 110 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSYLWPITFGQGTKVEIKRTV

SEQ ID NO: 103 - protein, 122 aa, Artificial Sequence
  Description of Artificial Sequence Synthetic polypeptide
EVQLVESGGGLVQPGGSLRLSCAASGFTVSSSSIHWVRQAPGKGLEWVASIYSYYGYTSYADSVKGRFTISADTSKNTAYLQMNSLRAEDTAVYYCARWQYHYNYWYGMDYWGQGTLVTVSS

SEQ ID NO: 104 - protein, 110 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYPMSLITFGQGTKVEIKRTV

SEQ ID NO: 105 - protein, 119 aa, Artificial Sequence
  Description of Artificial Sequence Synthetic polypeptide
EVQLVESGGGLVQPGGSLRLSCAASGFTVSYSSIHWVRQAPGKGLEWVASISPYSGYTSYADSVKGRFTISADTSKNTAYLQMNSLRAEDTAVYYCARGYDYYAGLDYWGQGTLVTVSS

SEQ ID NO: 106 - protein, 110 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYYYFPITFGQGTKVEIKRTV

SEQ ID NO: 107 - protein, 127 aa, Artificial Sequence
  Description of Artificial Sequence Synthetic polypeptide
EVQLVESGGGLVQPGGSLRLSCAASGFTFSYYSIHWVRQAPGKGLEWVASISPYYGYTSYADSVKGRFTISADTSKNTAYLQMNSLRAEDTAVYYCARWESEYSGTYEDYWAGMDYWGQGTLVTVSS

SEQ ID NO: 108 - protein, 110 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYMWWPITFGQGTKVEIKRTV

SEQ ID NO: 109 - protein, 121 aa, Artificial Sequence
  Description of Artificial Sequence Synthetic polypeptide
EVQLVESGGGLVQPGGSLRLSCAASGFTISYSSIHWVRQAPGKGLEWVASISSSYSYTSYADSVKGRFTISADTSKNTAYLQMNSLRAEDTAVYYCARTGYWQGYLALDYWGQGTLVTVSS

SEQ ID NO: 110 - protein, 110 aa, Artificial Sequence; identical to SEQ ID NO: 42
  Description of Artificial Sequence Synthetic polypeptide

SEQ ID NO: 111 - protein, 123 aa, Artificial Sequence
  Description of Artificial Sequence Synthetic polypeptide
EVQLVESGGGLVQPGGSLRLSCAASGFTISYSSIHWVRQAPGKGLEWVASISSSSGSTSYADSVKGRFTISADTSKNTAYLQMNSLRAEDTAVYYCARTYYYYWNSTPAMDYWGQGTLVTVSS

SEQ ID NO: 112 - protein, 110 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSYGYPVTFGQGTKVEIKRTV

SEQ ID NO: 113 - protein, 129 aa, Artificial Sequence
  Description of Artificial Sequence Synthetic polypeptide
EVQLVESGGGLVQPGGSLRLSCAASGFTFSSSSIHWVRQAPGKGLEWVASISSSYGYTSYADSVKGRFTISADTSKNTAYLQMNSLRAEDTAVYYCARWYNSSWYYSNWWYKGFGMDYWGQGTLVTVSS

SEQ ID NO: 114 - protein, 110 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYYSSLFTFGQGTKVEIKRTV

SEQ ID NO: 115 - protein, 124 aa, Artificial Sequence
  Description of Artificial Sequence Synthetic polypeptide
EVQLVESGGGLVQPGGSLRLSCAASGFTFYSSSIHWVRQAPGKGLEWVASISSSYGYTSYADSVKGRFTISADTSKNTAYLQMNSLRAEDTAVYYCARTSYTYPVYTYYGFDYWGQGTLVTVSS

SEQ ID NO: 116 - protein, 110 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSWYYPLTFGQGTKVEIKRTV

SEQ ID NO: 117 - protein, 122 aa, Artificial Sequence
  Description of Artificial Sequence Synthetic polypeptide
EVQLVESGGGLVQPGGSLRLSCAASGFTLYSSSIHWVRQAPGKGLEWVASISSSYGYTSYADSVKGRFTISADTSKNTAYLQMNSLRAEDTAVYYCARYRYSSWNRGAIDYWGQGTLVTVSS

SEQ ID NO: 118 - protein, 110 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSYWWPLTFGQGTKVEIKRTV

SEQ ID NO: 119 - protein, 122 aa, Artificial Sequence
  Description of Artificial Sequence Synthetic polypeptide
EVQLVESGGGLVQPGGSLRLSCAASGFTVSSSSIHWVRQAPGKGLEWVASIYSYYGYTSYADSVKGRFTISADTSKNTAYLQMNSLRAEDTAVYYCARWSKSPWYYQGIDYWGQGTLVTVSS

SEQ ID NO: 120 - protein, 112 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYHYWASLITFGQGTKVEIKRTV

SEQ ID NO: 121 - protein, 123 aa, Artificial Sequence
  Description of Artificial Sequence Synthetic polypeptide
EVQLVESGGGLVQPGGSLRLSCAASGFTVSSSSIHWVRQAPGKGLEWVASIYSYSGSTYYADSVKGRFTISADTSKNTAYLQMNSLRAEDTAVYYCARQYSSSYYVWPGMDYWGQGTLVTVSS

SEQ ID NO: 122 - protein, 112 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSYWWKSLVTFGQGTKVEIKRTV

SEQ ID NO: 123 - protein, 126 aa, Artificial Sequence
  Description of Artificial Sequence Synthetic polypeptide
EVQLVESGGGLVQPGGSLRLSCAASGFTLSSSSIHWVRQAPGKGLEWVASISSYYGSTSYADSVKGRFTISADTSKNTAYLQMNSLRAEDTAVYYCARMHYSWQEYYSYDWGMDYWGQGTLVTVSS

SEQ ID NO: 124 - protein, 110 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQPYYPLITFGQGTKVEIKRTV

SEQ ID NO: 125 - protein, 120 aa, Artificial Sequence
  Description of Artificial Sequence Synthetic polypeptide
EVQLVESGGGLVQPGGSLRLSCAASGFTISYSSIHWVRQAPGKGLEWVASIYPSYGSTSYADSVKGRFTISADTSKNTAYLQMNSLRAEDTAVYYCARWQGYYQPALDYWGQGTLVTVSS

SEQ ID NO: 126 - protein, 112 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSSKYYYPITFGQGTKVEIKRTV

SEQ ID NO: 127 - protein, 121 aa, Artificial Sequence
  Description of Artificial Sequence Synthetic polypeptide
EVQLVESGGGLVQPGGSLRLSCAASGFTISSSSIHWVRQAPGKGLEWVASISPYYGYTSYADSVKGRFTISADTSKNTAYLQMNSLRAEDTAVYYCARWGYGWYWYGLDYWGQGTLVTVSS

SEQ ID NO: 128 - protein, 111 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQGHDMNPVTFGQGTKVEIKRTV

SEQ ID NO: 129 - protein, 120 aa, Artificial Sequence
  Description of Artificial Sequence Synthetic polypeptide
EVQLVESGGGLVQPGGSLRLSCAASGFTLSSSSIHWVRQAPGKGLEWVASIYSSYGYTYYADSVKGRFTISADTSKNTAYLQMNSLRAEDTAVYYCARYYYYWYGGMDYWGQGTLVTVSS

SEQ ID NO: 130 - protein, 112 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSWMSDSLITFGQGTKVEIKRTV

SEQ ID NO: 131 - protein, 123 aa, Artificial Sequence
  Description of Artificial Sequence Synthetic polypeptide
EVQLVESGGGLVQPGGSLRLSCAASGFTFSYSSIHWVRQAPGKGLEWVASIYPSSGYTSYADSVKGRFTISADTSKNTAYLQMNSLRAEDTAVYYCARGWWYWMAWDYAMDYWGQGTLVTVSS

SEQ ID NO: 132 - protein, 112 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQMQYSGWLITFGQGTKVEIKRTV

SEQ ID NO: 133 - protein, 130 aa, Artificial Sequence
  Description of Artificial Sequence Synthetic polypeptide
EVQLVESGGGLVQPGGSLRLSCAASGFTISSSSIHWVRQAPGKGLEWVASISSYYGYTSYADSVKGRFTISADTSKNTAYLQMNSLRAEDTAVYYCARYYSYSSGYGYYDYFDWGAMDYWGQGTLVTVSS

SEQ ID NO: 134 - protein, 110 aa, Artificial Sequence; identical to SEQ ID NO: 42
  Description of Artificial Sequence Synthetic polypeptide

SEQ ID NO: 135 - protein, 129 aa, Artificial Sequence
  Description of Artificial Sequence Synthetic polypeptide
EVQLVESGGGLVQPGGSLRLSCAASGFTFSSSSIHWVRQAPGKGLEWVASISSSSGSTSYADSVKGRFTISADTSKNTAYLQMNSLRAEDTAVYYCARYYGYVWGGYWGWWYSKALDYWGQGTLVTVSS

SEQ ID NO: 136 - protein, 112 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYDWNYYLVTFGQGTKVEIKRTV

SEQ ID NO: 137 - protein, 126 aa, Artificial Sequence
  Description of Artificial Sequence Synthetic polypeptide
EVQLVESGGGLVQPGGSLRLSCAASGFTIYSSSIHWVRQAPGKGLEWVASISSYYGYTSYADSVKGRFTISADTSKNTAYLQMNSLRAEDTAVYYCARYQYYGSLYYSQQWAMDYWGQGTLVTVSS

SEQ ID NO: 138 - protein, 110 aa, Artificial Sequence; identical to SEQ ID NO: 42
  Description of Artificial Sequence Synthetic polypeptide

SEQ ID NO: 139 - protein, 129 aa, Artificial Sequence
  Description of Artificial Sequence Synthetic polypeptide
EVQLVESGGGLVQPGGSLRLSCAASGFTFSSSSIHWVRQAPGKGLEWVASISSSSGSTSYADSVKGRFTISADTSKNTAYLQMNSLRAEDTAVYYCARSPSSPYFMSWGWYWQYGIDYWGQGTLVTVSS

SEQ ID NO: 140 - protein, 111 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSSWGGLVTFGQGTKVEIKRTV

SEQ ID NO: 141 - protein, 121 aa, Artificial Sequence
  Description of Artificial Sequence Synthetic polypeptide
EVQLVESGGGLVQPGGSLRLSCAASGFTFSSSYIHWVRQAPGKGLEWVASISPSYGYTSYADSVKGRFTISADTSKNTAYLQMNSLRAEDTAVYYCARDMYEWWHWAIDYWGQGTLVTVSS

SEQ ID NO: 142 - protein, 110 aa, Artificial Sequence; identical to SEQ ID NO: 42
  Description of Artificial Sequence Synthetic polypeptide

SEQ ID NO: 143 - protein, 129 aa, Artificial Sequence
  Description of Artificial Sequence Synthetic polypeptide
EVQLVESGGGLVQPGGSLRLSCAASGFTFSSSSIHWVRQAPGKGLEWVASISSSSGSTSYADSVKGRFTISADTSKNTAYLQMNSLRAEDTAVYYCARYGHYLYYWGWGWYWSAALDYWGQGTLVTVSS

SEQ ID NO: 144 - protein, 514 aa, Artificial Sequence
  Description of Artificial Sequence Synthetic polypeptide
DIVRSDIQMTQSPSSLSASVGDRVTITCRASQSVSSAVAWYQQKPGKAPKLLIYSASSLYSGVPSRFSGSRSGTDFTLTISSLQPEDFATYYCQQISYVYSLITFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTFSSYSIHWVRQAPGKGLEWVASIYSSYGYTSYADSVKGRFTISADTSKNTAYLQMNSLRAEDTAVYYCARGGWYPAMDYWGQGTLVTVSSLEGGGGLNDIFEAQKIEWHESRHHHHHH

SEQ ID NO: 145 - protein, 517 aa, Artificial Sequence
  Description of Artificial Sequence Synthetic polypeptide
DIVRSDIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTISSSSIHWVRQAPGKGLEWVAYISSYSGYTYYADSVKGRFTISADTSKNTAYLQMNSLRAEDTAVYYCARYWYGHYHSYFGLDYWGQGTLVTVSSLEGGGGLNDIFEAQKIEWHESRHHHHHH

SEQ ID NO: 146 - protein, 512 aa, Artificial Sequence
  Description of Artificial Sequence Synthetic polypeptide
DIVRSDIQMTQSPSSLSASVGDRVTITCRASQSVSSAVAWYQQKPGKAPKLLIYSASSLYSGVPSRFSGSRSGTDFTLTISSLQPEDFATYYCQQYSYWPITFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTISSSYIHWVRQAPGKGLEWVAYISPSYGSTSYADSVKGRFTISADTSKNTAYLQMNSLRAEDTAVYYCAREYVTMALDYWGQGTLVTVSSLEGGGGLNDIFEAQKIEWHESRHHHHHH

SEQ ID NO: 147 - protein, 516 aa, Artificial Sequence
  Description of Artificial Sequence Synthetic polypeptide
DIVRSDIQMTQSPSSLSASVGDRVTITCRASQSVSSAVAWYQQKPGKAPKLLIYSASSLYSGVPSRFSGSRSGTDFTLTISSLQPEDFATYYCQQSSWGGLVTFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTFSSSYIHWVRQAPGKGLEWVASISPSYGYTSYADSVKGRFTISADTSKNTAYLQMNSLRAEDTAVYYCARDMYEWWHWAIDYWGQGTLVTVSSLEGGGGLNDIFEAQKIEWHESRHHHHHH

SEQ ID NO: 148 - protein, 514 aa, Artificial Sequence
  Description of Artificial Sequence Synthetic polypeptide
DIVRSDIQMTQSPSSLSASVGDRVTITCRASQSVSSAVAWYQQKPGKAPKLLIYSASSLYSGVPSRFSGSRSGTDFTLTISSLQPEDFATYYCQQISYVKKLITFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGGWYPAMDYWGQGTLVTVSSLEGGGGLNDIFEAQKIEWHESRHHHHHH

SEQ ID NO: 149 - protein, 232 aa, Artificial Sequence
  Description of Artificial Sequence Synthetic polypeptide
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 671 aa, Artificial Sequence
  Description of Artificial Sequence Synthetic polypeptide
EVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGGWYPAMDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 151 - protein, 446 aa, Artificial Sequence
  Description of Artificial Sequence Synthetic polypeptide
EVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGGWYPAMDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 152 - protein, 216 aa, Artificial Sequence
  Description of Artificial Sequence Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQISYVKKLITFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 153 - protein, 8 aa, Artificial Sequence
  Description of Artificial Sequence Synthetic peptide
ISYVKKLI

SEQ ID NO: 154 - protein, 8 aa, Artificial Sequence
  Description of Artificial Sequence Synthetic peptide
  MOD_RES (1)..(1): A, I, L, P, S, T, or V
  MOD_RES (2)..(2): K, R, S, or T
  MOD_RES (3)..(3): F or Y
  MOD_RES (4)..(4): I, R, T, or V
  MOD_RES (5)..(5): A, E, H, K, L, M, N, Q, R, S, T, or Y
  MOD_RES (6)..(6): K or R
  MOD_RES (7)..(7): A, C, D, E, G, H, K, L, M, N, P, Q, R, S, T, V, or W
  MOD_RES (8)..(8): I, L, or V
XXXXXXXX

SEQ ID NO: 155 - protein, 6 aa, Artificial Sequence
  Description of Artificial Sequence Synthetic peptide
GGWYPA

SEQ ID NO: 156 - protein, 6 aa, Artificial Sequence
  Description of Artificial Sequence Synthetic peptide
  MOD_RES (2)..(2): A, C, E, G, H, K, L, M, N, P, Q, R, S, T, W, or Y
  MOD_RES (3)..(3): G, P, R, T, or W
  MOD_RES (4)..(4): A, D, E, F, G, H, I, K, M, N, Q, R, S, T, W, or Y
  MOD_RES (5)..(5): A, P, or V
  MOD_RES (6)..(6): A, C, G, K, L, M, Q, R, S, T, or Y
GXXXXX

SEQ ID NO: 157 - protein, 6 aa, Artificial Sequence
  Description of Artificial Sequence Synthetic peptide
YSYWPI

SEQ ID NO: 158 - protein, 6 aa, Artificial Sequence
  Description of Artificial Sequence Synthetic peptide
  MOD_RES (2)..(2): A or S
  MOD_RES (3)..(3): A, C, D, E, F, G, H, I, K, L, M, N, Q, R, S, T, V, W, or Y
  MOD_RES (6)..(6): E, I, or P
YXXWPX

SEQ ID NO: 159 - protein, 7 aa, Artificial Sequence
  Description of Artificial Sequence Synthetic peptide
EYVTMAL

SEQ ID NO: 160 - protein, 7 aa, Artificial Sequence
  Description of Artificial Sequence Synthetic peptide
  MOD_RES (1)..(1): D or E
  MOD_RES (2)..(2): E, F, L, Q, S, W, or Y
  MOD_RES (3)..(3): I, T, or V
  MOD_RES (4)..(4): A, E, N, S, or T
  MOD_RES (5)..(5): F, M, or Y
  MOD_RES (6)..(6): A, C, G, S, or T
  MOD_RES (7)..(7): A, C, E, F, H, I, K, L, M, N, P, Q, S, T, or V
XXXXXXX

SEQ ID NO: 161 - protein, 488 aa, Artificial Sequence
  Description of Artificial Sequence Synthetic polypeptide
DIVRSDIQMTQSPSSLSASVGDRVTITCRASQSVSSAVAWYQQKPGKAPKLLIYSASSLYSGVPSRFSGSRSGTDFTLTISSLQPEDFATYYCQQISYVYSLITFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTFSSYSIHWVRQAPGKGLEWVASIYSSYGYTSYADSVKGRFTISADTSKNTAYLQMNSLRAEDTAVYYCARGGWYPAMDYWGQGTLVTVSSLE

SEQ ID NO: 162 - protein, 491 aa, Artificial Sequence
  Description of Artificial Sequence Synthetic polypeptide
DIVRSDIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTISSSSIHWVRQAPGKGLEWVAYISSYSGYTYYADSVKGRFTISADTSKNTAYLQMNSLRAEDTAVYYCARYWYGHYHSYFGLDYWGQGTLVTVSSLE

SEQ ID NO: 163 - protein, 486 aa, Artificial Sequence
  Description of Artificial Sequence Synthetic polypeptide
DIVRSDIQMTQSPSSLSASVGDRVTITCRASQSVSSAVAWYQQKPGKAPKLLIYSASSLYSGVPSRFSGSRSGTDFTLTISSLQPEDFATYYCQQYSYWPITFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTISSSYIHWVRQAPGKGLEWVAYISPSYGSTSYADSVKGRFTISADTSKNTAYLQMNSLRAEDTAVYYCAREYVTMALDYWGQGTLVTVSSLE

SEQ ID NO: 164 - protein, 490 aa, Artificial Sequence
  Description of Artificial Sequence Synthetic polypeptide
DIVRSDIQMTQSPSSLSASVGDRVTITCRASQSVSSAVAWYQQKPGKAPKLLIYSASSLYSGVPSRFSGSRSGTDFTLTISSLQPEDFATYYCQQSSWGGLVTFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTFSSSYIHWVRQAPGKGLEWVASISPSYGYTSYADSVKGRFTISADTSKNTAYLQMNSLRAEDTAVYYCARDMYEWWHWAIDYWGQGTLVTVSSLE

SEQ ID NO: 165 - protein, 488 aa, Artificial Sequence
  Description of Artificial Sequence Synthetic polypeptide
DIVRSDIQMTQSPSSLSASVGDRVTITCRASQSVSSAVAWYQQKPGKAPKLLIYSASSLYSGVPSRFSGSRSGTDFTLTISSLQPEDFATYYCQQISYVKKLITFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFTFSDYSIHWVRQAPGKGLEWVASISSSSGSTSYADSVKGRFTISADTSKNTAYLQMNSLRAEDTAVYYCARGGWYPAMDYWGQGTLVTVSSLE